(19) United States
(12) Patent Application Publication
(10) Pub. No.: US 2024/0165311 A1
(43) Pub. Date: May 23, 2024

(54) STEM CELLS FOR USE IN ECMO TECHNOLOGY

(71) Applicant: Amniotics AB, Lund (SE)

(72) Inventors: Marcus Larsson, Lund (SE); Sandra Lindstedt Ingemansson, Lund (SE)

(21) Appl. No.: 18/551,048

(22) PCT Filed: Mar. 18, 2022
(86) PCT No.: PCT/EP2022/057252

§ 371 Date: Sep. 18, 2023

(30) Foreign Application Priority Data
Mar. 19, 2021 (GB) 2103889.8

(51) Int. Cl. A61M 1/16; A61B 5/00; A61B 5/145; A61K 35/28; A61M 1/36

(52) U.S. Cl. CPC A61M 1/1698; A61B 5/14542; A61B 5/6866; A61K 35/28; A61M 1/3666; A61M 2230/205

(57) ABSTRACT

The present invention relates to the field of stem cells and their use in processing ex vivo samples (e.g. blood samples). More specifically, the present invention relates to the use of mesenchymal stem cells in ECMO.

### FIELD OF INVENTION

The present invention relates to the field of stem cells and their use in processing ex vivo samples (e.g. blood samples). More specifically, the present invention relates to the use of mesenchymal stem cells in ECMO.

### BACKGROUND

Extracorporeal membrane oxygenation (ECMO) is an extracorporeal technique for providing prolonged cardiac and respiratory support in subjects that are unable to provide an adequate amount of gas exchange or perfusion to sustain life. ECMO is an established tool for supporting those with refractory illness, particularly in Severe Acute Respiratory Distress Syndrome (ARDS). However, while the survival benefits for ECMO have been recognised, further advancements are necessary to increase confidence in the clinic for placing subjects on ECMO.

Mesenchymal stem cells (MSCs) can be found in nearly all tissues and are mostly located in perivascular niches. As will be understood by one of skill in the art, MSCs are multipotent stromal cells capable of differentiating into numerous cell types, and also possessing anti-inflammatory, angiogenic properties for directing tissue repair processes, thereby making MSCs valuable for therapeutic treatments. Term amniotic fluid (TAF) collected during a caesarean section contains a number of valuable cells, including MSCs. Moreover, specific subpopulations of MSCs are likely to be particularly well suited to use for production of therapeutic drugs. Previously, MSCs sourced from adult bone marrow, adult adipose tissue or neonatal birth-associated tissues including placenta, umbilical cord and cord blood were extensively used to obtain MSCs. MSCs from these neonatal tissues may have additional capacities in comparison to MSCs derived from adult sources. Indeed, several studies have reported superior biological properties such as improved proliferative capacity, life span and differentiation potential of MSCs from birth-associated tissues over adult derived MSCs. However, neither of these neonatal MSC sources have a corresponding tissue or organ in the adult body. Therefore, a neonatal quality MSCs with tissue specificity would be extremely beneficial. Moreover, acquisition of fetal material may be linked to negative consequences for the infant. For example, in cord blood harvesting it has been shown that as much of the cord blood as possible should be returned to the infant for improved survival, growth and fine motor skills development.

Although MSCs have been tested in ECMO, their use raises a number of concerns. For example, MSCs are large cells that may reduce patency of the oxygenation membrane in ECMO; and MSCs are characteristically sticky to plastic, a key component in ECMO devices (Millar et al., 2020), and previous studies have confirmed that this results in limitations of MSC use in ECMO (Millar et al., 2019). Millar et al., 2020 further identified that despite endobronchial administration, the hMSCs used adhered to and impacted the commercial membrane oxygenator function in vivo, with an enhanced trans-membrane oxygenator pressure gradient being observed.

However, due to the anti-inflammatory properties of MSCs, their use in ECMO remains an attractive option, provided that the problem of membrane clogging by MSCs can be addressed.

### SUMMARY OF INVENTION

Accordingly, it is an objection of the present invention to provide alternative and improved types of MSCs for use in ECMO. The type of MSCs characterised herein are derived from term amniotic fluid (TAF MSCs), which have been further characterised by panels of markers to identify a lung-specific subset of TAF MSCs. Term amniotic fluid (TAF) collected during a caesarean section contains a number of valuable cells, including MSCs. Amniotic fluid is today considered medical waste that is discarded. Therefore, both the ethical and practical incentive to harvest such an untapped resource is clear.

A first aspect of the invention relates to isolated term amniotic fluid (TAF) mesenchymal stem cells (MSCs) for use in treating low blood oxygenation levels in a subject via extracorporeal membrane oxygenation (ECMO).

A second aspect of the invention relates to a composition comprising isolated term amniotic fluid (TAF) mesenchymal stem cells (MSCs) for use in treating low blood oxygenation levels in a subject via extracorporeal membrane oxygenation (ECMO).

A third aspect of the invention relates to a method of treating a condition associated with low blood oxygenation (e.g. ARDS) using isolated TAF MSCs and/or a composition comprising isolated TAF MSCs in a subject via ECMO.

A fourth aspect of the invention relates to use of isolated TAF MSCs and/or a composition comprising isolated TAF MSCs for the manufacture of a medicament for the treatment of a condition associated with low blood oxygenation (e.g. ARDS).

A fourth aspect of the invention relates to isolated TAF MSCs for use in treating acute respiratory distress syndrome (ARDS) in a subject via ECMO.

A fifth aspect of the invention relates to a composition comprising isolated TAF MSCs for use in treating ARDS in a subject via ECMO.

A sixth aspect of the invention relates to an extracorporeal blood treatment system for treating a patient, the system comprising:
an extracorporeal blood circuit;
a processing fluid circuit;
said extracorporeal blood circuit and processing fluid circuit being divided by an oxygenation membrane of a filtration unit;
at least one blood pump for controlling the flow of blood through the blood circuit;
at least one processing fluid pump for controlling the flow of processing fluid through the processing fluid circuit;
a system computing unit operatively connected to the blood pump and the processing fluid pump, said system computing unit having at least one input means (e.g. a keyboard, touch screen or sensor); wherein
the system computing unit is adapted for receiving a desired blood oxygenation value O_{b};
the system computing unit is adapted for receiving an actual blood oxygenation value Oₐ;
the system computing unit being adapted for controlling said blood pump and said processing fluid pump so as the actual blood oxygenation value Oₐ is driven towards the desired blood oxygenation value O_{b};
the system is adapted for receiving isolated TAF MSCs.

A seventh aspect of the invention relates to a formulation for use in the system of the sixth aspect, wherein the formulation comprises isolated TAF MSCs and dimethyl sulfoxide DMSO.

An eighth aspect of the invention relates to a method of oxygenating a blood sample in the presence of isolated TAF MSCs.

### DESCRIPTION OF THE FIGURES

FIG. 1: A flow diagram showing the steps in the purification, culturing and selection of MSC subpopulations.

FIG. 2: A diagram illustrating a method for collecting amniotic fluid.

FIG. 3: A schematic illustration, in a perspective view, of an apparatus for filtering amniotic fluid according to an example.

FIG. 4: A schematic illustration, in a cross-sectional side view, of an apparatus for filtering amniotic fluid according to an example.

FIG. 5: A schematic illustration, in a cross-sectional side view, of an apparatus for filtering amniotic fluid according to an example.

FIG. 6: A schematic illustration, in a cross-sectional side view, of an apparatus for filtering amniotic fluid according to an example.

FIG. 7: A schematic illustration, in a cross-sectional side view, of an apparatus for filtering amniotic fluid according to an example.

FIG. 8: A schematic illustration, in a cross-sectional side view, of an apparatus for filtering amniotic fluid according to an example.

FIG. 9: A schematic illustration, in a cross-sectional side view, of an apparatus for filtering amniotic fluid according to an example.

FIG. 10: A schematic illustration, in a cross-sectional side view, of an apparatus for filtering amniotic fluid according to an example.

FIG. 11: A schematic illustration, in a cross-sectional side view, of an apparatus for filtering amniotic fluid according to an example.

FIG. 12: (a) A schematic illustration, in a cross-sectional side view, of an apparatus for filtering amniotic fluid according to an example. (b) A schematic illustration, along a cross-section A-A in FIG. 10, of an apparatus for filtering amniotic fluid according to an example.

FIG. 13: A flow chart of a method of filtering amniotic fluid according to an example.

FIG. 14: A flow chart showing the steps for calculation of an MSC tissue specificity score according to an example.

FIG. 15: An example graph showing MSC tissue specificity scores representing the 5% and 15% thresholds.

FIG. 16: An example graph showing tissue-prioritized and tissue-distal data, including tissue-prioritized data greater than 15% percentile.

FIG. 17: (A)-(D) show the results of an example study demonstrating the effects of using TAF Lung MSCs to treat rats with induced lung fibrosis.

FIG. 18: Cell number over time. The x-axis shows time in minutes and the y-axis shows number of cells. The darker grey line represents total amount of cells and the lighter grey line represents live cells.

FIG. 19: (Table 1) Plate layout—Two plates of each layout were prepared. One plate for cell composition and cytokine (FACS/Luminex) analysis and one plate for cell composition and proliferation (CFSE) analysis. PBMCs and MSCs were added at indicated ratios to columns 1, 2, 3, 6, 7, 8, 11, 12 but not to columns 4, 5, 9, 10.

FIG. 20: Gating strategy used for all samples, analysed after 24 hours of activation, to identify specific PBMC subpopulations and to exclude MSCs. The figure shows gating strategy for PBMC:MSC sample (1:2.5) activated with aCD3/aCD2B in cell composition plates. A) Gating of single cells, B) gating of lymphocytes and granulocytes, C) NOT gating on non T cells, D) gating of CD4+ and CD8+ cells among T cells, E) gating of PD-1+ and CD73+ cells among CD4+ lymphocytes (double positives are not included), F) gating of PD-1+ and CD73+ cells among CD8+ lymphocytes (double positives are not included), G) gating of PD-1+ and CD73+ cells among CD4+ lymphocytes, H) gating of PD-1+ and CD73+ cells among COB+ lymphocytes, I) gating of CD80+ cells among granulocytes and macrophages, J) gating of CD73+ cells among CD80+ granulocytes and macrophages, K) gating of CD206+ cells among granulocytes and macrophages, L) gating of CD163+ cells among CD206+ granulocytes and macrophages and M) gating of CD73+ cells among CD163+CD206+ granulocytes and macrophages. Back-gating was performed to verify lymphocyteand granulocyte/macrophage gates.

FIG. 21: Representative figures showing gating of CD4+ and CD8+ cells among T cells for all PBMC:MSC ratios. (FSC vs SSC and CD4 vs CD8) for PBMC:MSC ratios 1:0, 1:2.5, 1:5, 1:10 and 0:1, activated for 24 hours with aCD3/aCD28 in the cell composition plate. From the FSC vs SSC dot plot-lymphocyte gate, a NOT gate was set on non-T cells and CD4+ and CD8+ T cells were analysed.

FIG. 22: T cell activation status after co-culturing PBMCs with MSCs or reference drugs. PBMCs and MSCs were co-cultured in different PBMC:MSC ratios and activated with aCD3/aCD28. Cell populations were analysed using flow cytometry after 24 hours of incubation. Results show A) % CD4+ among lymphocytes, B) Expression of CD4 on T cells (MFI), C) % PD-1+ among CD4+ lymphocytes, D) Expression of PD-1 on CD4+ lymphocytes (MFI), E) % PD-1+ among CD4+ lymphocytes (including double positives), F) Expression of PD-1 on CD4+ lymphocytes (MFI) (including double positives), G) % CD73+ among CD4+ lymphocytes, H) Expression of CD73 on CD4+ lymphocytes (MFI), % CD73+ among CD4+ lymphocytes (including double positives), J) Expression of CD73 on CD4+ lymphocytes (MFI) (including double positives), K) % CD8 among lymphocytes, L) Expression of CD8 on T cells (MFI), M) % PD-1+ among CD8+ lymphocytes, N) Expression of PD-1 on CD8+ lymphocytes (MFI), 0) % PD-7+ among CD8+ lymphocytes (including double positives), P) Expression of PD-1 on CD8+ lymphocytes (MFI) (including double positives), Q) % CD73+ among CD8+ lymphocytes, R) Expression of CD73 on CD8+ lymphocytes (MF1), S) % CD73+ among CD8+ lymphocytes (including double positives) and T) Expression of CD73 on CD8+ lymphocytes (MFI) (including double positives). Results are presented as mean values (of % or median fluorescent intensity)+/−SEM.

FIG. 23: Macrophage activation status after coculturing PBMCs with MSCs or reference drugs. PBMCs and MSCs were co-cultured in different PBMC:MSC ratios and activated with aCD3/aCD28. Cell populations were analysed using flow cytometry after 24 hours of incubation. Results show A) % CD80+ among granulocytes and macrophages, B) Expression of CD80 on granulocytes and macrophages (MR), C) % CD73+ among CD80+ granulocytes and macrophages, D) Expression of CD73 on CD80+ granulocytes and macrophages (MEI), E) % CD163+CD206+ among granulocytes and macrophages, F) Expression of CD163 and CD206 on granulocytes and macrophages (MEI), G) % CD73+ among CD163+CD206+ granulocytes and macrophages, H) Expression of CD73 on CD163+CD206+ granulocytes and macrophages, Results are presented as mean values (of % or median fluorescent intensity)+/−SEM.

FIG. 24: Gating strategy used for all samples, analysed after 72 hours of activation, to identify specific PBMC subpopulations and to measure proliferation using CFSE labeling. CFSE labeling of PBMCs was performed before coculture with MSCs, therefore all CFSE positive cells are PBMCs. The figure shows gating strategy for PBMC:MSC sample (1:2.5) activated with aCD3/aCD28 in CFSE proliferation plates. A) Gating of single cells and B) gating of lymphocytes and granulocytes/macrophages. Gating for CD4+ cells and CD8+ cells was performed as for the 24-hour activation samples. Back-gating was performed to verify lymphocyte and granulocyte/macrophage gates.

FIG. 25: Histograms showing the gating strategy for CFSE plates. A) Gating of CFSE proliferation among CD4+ lymphocytes, B) gating of CFSE proliferation among CD8+ lymphocytes, C) gating of CFSE proliferation among CD80+ granulocytes and macrophages and D) gating of CFSE proliferation among CD163+CD206+ granulocytes and macrophages. Horizontal bars delineate proliferating cells. E) shows CFSE zero peak.

FIG. 26: Representative graphs of CFSE proliferation in CD4+ PBMCs co-cultured with MSCs in media 3 and analysed using flow cytometry after 72 hours of incubation. PBMCs and MSCs were co-cultured in different ratios (PBMC:MSC—0:1, 1:10, 1:5, 1:2.5, and 1:0).

FIG. 27: A) Representative graphs of CFSE proliferation in CD4+ PBMCs co-cultured with MSCs in media 3 and analysed using flow cytometry after 72 hours of incubation. PBMCs and MSCs were co-cultured in different ratios (PBMC:MSC—0:1, 1:10, 1:5, 1:2.5, and 1.0). B) Representative graphs of CFSE proliferation in CD8+ PBMCs co-cultured with MSCs in media 3 and analysed using flow cytometry after 72 hours of incubation. PBMCs and MSCs were co-cultured in different ratios (PBMC:MSC—0:1, 1:10, 1:5, 1:2.5, and 1:0). C) and D) Representative graphs of CFSE proliferation in CD80+ cells (M1) and in CD163+CD206+ cells (M2), analysed using flow cytometry after 72 hours of incubation. Representative graphs show PBMCs and MSCs co-cultured in different ratios (PBMC:MSC—0:1, 1:10, 1:5, 1:2.5 and 1:0) cultured in media 3.

FIG. 28: A) and B) Representative graphs of CFSE proliferation in CD80+ cells (M1) and in CD163+CD206+ cells (M2), analysed using flow cytometry after 72 hours of incubation. Representative graphs show PBMCs and MSCs co-cultured in different ratios (PBMC:MSC—0:1, 1:10, 1:5, 1:2.5 and 1:0) cultured in media 3.

FIG. 29: T cell composition and proliferation status after coculturing PBMCs with MSCs or reference drugs. PBMCs and MSCs were co-cultured in different PBMC:MSC ratios and activated with aCD3/aCD28. Cell populations were analysed using flow cytometry after 72 hours of incubation. Results show A) % CD4+ among T cells, B) Expression of CD4 on T cells (MFI), C) Proliferating CD4+ among T cells, D) MSCs inhibitory effect on CD4+ cells, E) % CD8+ among T cells, F) Expression of CD8 on T cells (MFI), G) Proliferating CD8+ among T cells and H) MSCs inhibitory effect on CD8+ cells. Results are presented as mean values+/−SEM. CFSE zero peak has been removed from analysis. Proliferation has been analysed using the geometric mean (Geo mean) value which describes the MFI (mean fluorescent intensity) in a logarithmic histogram. The number of events in each fluorescent channel is divided by the number of channels but since the scale is logarithmic, arithmetic values cannot be used. The Geo mean compensates for the logarithmic scale and is also considering bright and dim populations.

FIG. 30: Macrophage composition and proliferation status after coculturing PBMCs with MSCs or reference drugs. PBMCs and MSCs were co-cultured in different PBMC:MSC ratios and activated with aCD3/aCD28. Cell populations were analysed using flow cytometry after 72 hours of incubation. Results show A) % CD80+ among granulocytes and macrophages, B) Expression of CD80 on granulocytes and macrophages (MFI), C) Proliferating CD80+ among granulocytes and macrophages, D) MSCs inhibitory effect on CD80+ cells, E) % CD163+CD206+ among granulocytes and macrophages, F) Expression of CD163 and CD206 on granulocytes and macrophages (MFI), G) Proliferating CD163+CD206+ among granulocytes and macrophages and H) MSCs inhibitory effect on CD163+CD206+ cells. Results are presented as mean values (of % or median fluorescent intensity)+/−SEM. Proliferation has been analysed using the geometric mean (Geo mean) value which describes the MFI (mean fluorescent intensity) in a logarithmic histogram. The number of events in each fluorescent channel is divided by the number of channels but since the scale is logarithmic, arithmetic values cannot be used. The Geo mean compensates for the logarithmic scale and is also considering bright and dim populations.

FIG. 31: Cytokine analysis after co-culturing PBMCs with MSCs or reference drugs. PBMCs and MSCs were co-cultured in different PBMC:MSC ratios and activated with aCD3/aCD28. Cytokine levels in supernatants from cells in FACS/Luminex plates were analysed using Luminex after 24 hours of incubation. Results show levels of A) IGF, B) CXCL9, C) IL-10, D) IFN-α, E) HGF, F) IL-6 (FI), G) IL-18, H) IFN-g, I) VEGF (FI), J) TNF-α, K) IL-12/IL-23p40, L) b-NGF, in all tested PBMC:MSC ratios in media 3. Results are presented as mean values+/−SEM. For some of the analysed cytokines, IFN-α values are below the dynamic range and IL-6 and VEGF values are above the dynamic range and results are therefore presented as FI levels (fluorescence intensity).

FIG. 32: Cytokine levels were analysed in supernatants from aCD3/aCD28 activated PBMCs alone, MSCs alone and in CM from all donors using Luminex after 24 hours of incubation. Results show A) CXCL9 levels, B) TNF-α levels, C) IFN-α levels, D) IGFBP-1 levels, E) IL-10 levels, F) IL-18 levels, G) HGF levels, H) IFN-g levels, I) IL-6 levels, J) IL-12/23 levels, K) b-NGF levels and L) VEGF levels in supernatant using media 3. Results are presented as mean values+/−SEM. For some of the analysed cytokines, IFN-α values are below the dynamic range and IL-6 and VEGF values are above the dynamic range and results are therefore presented as FI levels (fluorescence intensity).

### DETAILED DESCRIPTION OF THE INVENTION

In some embodiments, the use in treating low blood oxygenation levels in a subject treats respiratory distress (e.g. Acute Respiratory Distress Syndrome (ARDS)) and/or hypoxemic respiratory failure. In some embodiments, the use in treating low blood oxygenation levels in a subject is to provide prolonged cardiac and respiratory support to subjects whose heart and lungs are unable to provide an adequate amount of gas exchange or perfusion to sustain life.

Isolated TAF MSCs can freely pass an oxygenator membrane, and so could be added before and/or after the membrane in an ECMO system.

In some embodiments, the isolated TAF MSCs are administered before ECMO. In some embodiments, the isolated TAF MSCs are administered during ECMO. In some embodiments, the isolated TAF MSCs are administered after ECMO. In some embodiments, the isolated TAF MSCs are administered before, during and/or after ECMO.

By “before ECMO” we refer to the isolated TAF MSCs being introduced to an ECMO system prior to a blood sample meeting the oxygenator membrane. We also include the meaning that the isolated TAF MSCs may be introduced to a subject in need of ECMO prior to an ECMO procedure commencing. Alternatively, or additionally, the isolated TAF MSCs may be introduced to an ECMO system before a subject is placed on the system, so as to prime an ECMO system with isolated TAF MSCs before the subject commences ECMO treatment.

By “during ECMO” we refer to the isolated TAF MSCs being introduced to an ECMO system while ECMO treatment is ongoing, for example mixed with the blood sample upon it contacting the oxygenator membrane. Isolated TAF MSCs may be added during ECMO to supplement the amount introduced before ECMO. Isolated TAF MSCs may be introduced during ECMO to maintain a minimum threshold of isolated TAF MSCs in the system.

By “after ECMO” we refer to the isolated TAF MSCs being introduced to an ECMO system after the blood sample has contacted the oxygenator membrane. We also include the meaning that the isolated TAF MSCs may be introduced to a subject who has recently undergoing an ECMO treatment.

In some embodiments, the use in treating low blood oxygenation levels in a subject diagnosed with Acute Respiratory Distress Syndrome (ARDS). ARDS is typically induced by either known or unknown environmental factors including viral (such as SARS-CoV-2, the virus that causes COVID-19) or bacterial infection that induces pulmonary tissue damage and inflammatory responses. As of yet, no drug or vaccine has been clearly shown to cure patients with COVID-19, therefore there is a need for new treatments. In particular, modulating and/or reducing the well-documented and potentially lethal “cytokine storm” inflammatory response in COVID-19 patients may improve patient health and survival.

Lung TAF MSCs, such as those described herein, may be uniquely suited for the treatment of patients with low blood oxygenation, such as patients with ARDS. Lung TAF MSCs may be suitable for the treatment of a variety of acute and/or chronic respiratory diseases. Additionally, Lung TAF MSCs and TAF MSCs generally are also known to be smaller than conventional MSCs, thereby making them more suitable for intravenous dosed treatments. “Morphology and size of stem cells from mouse and whale: observational study” by Hoogdujin et al. and “The size of mesenchymal stem cells is a significant cause of vascular obstructions and stroke.” by Ge et al. provide further details regarding the relative size of MSCs and their potential reduced role in vascular obstructions or stroke.

For example, Lung TAF MSCs may have an anti-inflammatory effect on other cell types, such as cells found within various organs and tissues such as the lung. Therefore, incorporation of Lung TAF MSCs within the lung of a patient suffering from an acute and/or chronic respiratory disease may reduce inflammation. Lung TAF MSCs may be particularly beneficial to patients suffering from ARDS caused by COVID-19 because the Lung TAF MSCs may reduce the magnitude of the dangerous “cytokine storm” induced in COVID-19 patients. Additionally, in some examples, Lung TAF MSCs express lower levels of the ACE/ACE2 receptor as compared to adult bone marrow and adipose MSCs, indicating that SARS-CoV-2 may be less likely to infect Lung TAF MSCs as compared to other MSCs. Further, TAF MSCs (including lung TAF MSCs) have been shown to reduce cytokine responses such as IL-6, IL-18, and TNF-α etc., as well as generally lower the activation and proliferation of lymphocytes (T-cell, macrophages etc) and increase levels of several growth factors.

In some embodiment, the use, method and/or systems described herein increase the oxygenation of a subject in comparison with a control, such as a control subject that is not exposed to TAF MSCs via ECMO, or a control reading that is taken prior to ECMO being performed.

In some embodiment, blood oxygen levels are determined before, during and/or after ECMO from a sample obtained from a subject. Blood oxygen can be determined using techniques known in the art, for example by an arterial blood gas test. A higher blood oxygen level on a sample tested after ECMO compared with a sample obtained before and/or during ECMO (or compared with the average level of a healthy person, wherein normal is considered to be between 75-100 mmHg, and <60 mmHg is considered low) is indicative of successful ECMO treatment. Accordingly, a comparison with a control that excludes TAF MSCs may be indicative of an improvement in successful ECMO treatment, where blood oxygen levels are higher in the TAF MSC condition than the control. For example, blood oxygen level may increase in the TAF MSC condition compared with a control lacking TAF MSCs by 5 mmHg, preferably 10, 20, 30 or more mmHg. This increase in blood oxygen level may be detected within 30 minutes, 1 hour, 2 hours, 3 hours, 4 hours or more of ECMO.

In some embodiments, the ECMO system is adapted for receiving 10 million, million, 30 million, 40 million, 50 million, 60 million, 70 million, 80 million, 90 million, 100 million or more isolated TAF MSCs per minute. In a preferred embodiment, the ECMO system is adapted for receiving 20 million isolated TAF MSCs per minute.

The number of TAF MSCs used in the system preferably does not exceed a flow of 20×10⁶ TAF MSCs per minute, after which there is an increased risk of thrombosis. However, this can be mitigated, and therefore the cellular concentration increased, where further components such as an anticoagulant (e.g. heparin, such as low molecular weight heparin) are included to lower the risk of thrombosis. The capacity for cells can be adjusted based on the subject receiving ECMO. For example, if the flow is 20×10⁶ TAF MSCs per minute and the subject should be treated with 2 million per kg, the subject should receive 140×10⁶ TAF MSCs over 7 minutes. The concentration of components such as DMSO are similarly constrained to not exceed a particular level in the subject. For example, although DMSO concentration may not impact flow, care should be taken to not exceed 1 mL of DMSO per kg of subject weight per day.

In some embodiments, the number of TAF MSCs used in the system does not cause clogging of an oxygenator membrane. This can in particular be seen in Example 1. By “clogging” we include the meaning that an accumulation of cellular content (i.e. MSCs or TAF MSCs) occurs at a contact site of an oxygenator membrane either as single cells or as aggregates, thereby impeding the flow and/or increasing the pressure of said membrane. In the present context the term “aggregates” is to be understood as 10 or more cells linked together. The terms “clogging”, “blocking”, “impeding” and “obstructing” are used herein interchangeably. In some embodiments, the TAF MSCs do not cause clogging after 1, 2, 3, 4, 5, 6, 7, 8, 9, 10, 11, 12, 13, 14, 15, 16, 17, 18, 19, 20, 21, 22, 23, 24 or more hours of ECMO. For example, the TAF MSCs do not cause clogging after more than 4 hours of ECMO. In some embodiments, the level of clogging (as determined by membrane pressure) is reduced in the system that uses TAF MSCs as compared with a control, for example wherein the control is adult MSCs (referring to MSCs derived from an adult source). This may be expressed as a percentage of membrane clogging or as a ratio of membrane clogging in comparison with a control. For example, the use of TAF MSCs may reduce clogging compared with adult MSCs by 10%, 20%, 30%, 40%, 50%, 60%, 70%, 80%, 90°/a or 100% following, for example, 4 hours of ECMO.

In some embodiments, the formulation of TAF MSCs and DMSO is reconstituted before application in ECMO. For example, the TAF MSCs and DMSO may be reconstituted in a buffering fluid. The buffering fluid may be a solution known in the art for use in ECMO.

Isolated TAF MSCs may be introduced to an ECMO system at any point within the system. For example, the isolated TAF MSCs may be introduced via the same fluid processing pump that controls the flow of processing fluid through the processing fluid circuit. Alternatively, or additionally, the isolated TAF MSCs may be introduced via a separate (i.e. additional) fluid processing pump that controls the flow of a buffering fluid through a different part of the processing fluid circuit, wherein the buffering fluid combines with the processing fluid prior to, at the moment of, and/or after the fluid contacts the oxygenator membrane. Accordingly, the ECMO system may be adapted for receiving isolated TAF MSCs before and/or after the oxygenation membrane.

The desired blood oxygenation value may be a minimum value, a maximum value, a therapeutically effective value, a value deemed to be within a physiologically healthy range (which may be assessed based on the subject), or combinations thereof. For example, a minimum level may be the minimum amount that is deemed healthy for a subject that is at an unhealthy blood oxygenation level, and so the system computing unit is adapted for driving the actual blood oxygenation level of the subject towards the minimum healthy blood oxygenation level.

In some embodiments, the system computing unit is adapted for receiving at least one desired blood concentration of isolated TAF MSCs (for example, the system computing unit may be adapted for receiving a desired maximum blood concentration of isolated TAF MSCs and/or a desired minimum blood concentration of isolated TAF MSCs). Said system computing unit may therefore be configured to reduce the number of isolated TAF MSCs in the system upon detection of a cell concentration above the desired maximum blood concentration (e.g. by halting introduction of further cells and/or by siphoning off existing cells in the system). Additionally, or alternatively, said system may be configured to increase the number of isolated TAF MSCs in the system upon detection of a cell concentration below the desired minimum blood concentration (e.g. by halting the siphoning off of existing cells in the system and/or by activating a processing fluid circuit operatively linked to the introduction of a fluid containing isolated TAF MSCs).

In some embodiments, the system computing unit is adapted for detecting that a desired maximum blood concentration of isolated TAF MSCs has been met by, for example, a cell count and/or membrane pressure. For example, the system computing unit may include a Nucleocounter-202 (NC-202) for counting the number of isolated TAF MSCs. The NC-202 may be part of a separate channel, wherein a sample present in the ECMO system is siphoned off and treated as per Example 1. Alternatively, or additionally, the system computing unit may be adapted for detecting membrane pressure, which increases in proportion to the number of MSCs present in a system. Although TAF MSCs are significantly less prone to causing increases in membrane pressure, systems adapted for use with alternative types of processing fluids (e.g. presence and levels of heparin) may have other components that impact membrane stickiness, and so it may be beneficial to include a membrane pressure sensor.

In some embodiments, the system is adapted for receiving an anticoagulant (e.g. heparin). In some embodiments, the system is further adapted for altering the level of anticoagulant (e.g. heparin). For example, the system may be configured to assess activated clotting time (ACT), activated partial thromboplastin time (aPTT), anti-Xa assays, and/or viscoelastic testing, as described further in Cho et al., 2017.

In some embodiments, the isolated TAF MSCs are comprised in a formulation for use in an ECMO system, such as the systems described herein. In preferred embodiments, the formulation comprises 10×10⁶ cells/mL and 10% DMSO. In some embodiments, the formulation further comprises additional components, such as buffering components, pharmaceutically acceptable carriers, and/or anticoagulants. In some embodiments, the formulation is in a form that requires it to be reconstituted before application in ECMO, for example in a buffering fluid as described herein.

In some embodiments, the number of isolated TAF MSCs is at least 1 million cells per kg of subject, for example at least 2, 3, 4, 5, 6, 7, 8, 9, 10, 11, 12, 13, 14, 15, 16, 17, 18, 19 or 20 million cells per kg of the subject. Preferably, the number of isolated TAF MSCs is from 1-4 million cells per kg of subject, for example 2 million cells per kg of subject. Preferably, the number of isolated TAF MSCs it at a level per kg of the subject that does not exceed the maximum desired cell concentration in an ECMO system. Accordingly, in a particularly preferred embodiment, the flow of isolated TAF MSCs in the ECMO system is about 20 million cells per minute, which equates to between 1-4 million cells per kg of subject, even more preferably 2 million cells per kg of subject.

As used herein, a “subject” means a human or animal. Usually, the animal is a vertebrate such as a primate, rodent, domestic animal, or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include pigs, cows, horses, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, pig, mouse, rat, dog, cat, horse, or cow, but are not limited to these examples. In an embodiment, the subject is human. In addition, the uses, systems and methods described herein can be used to treat domesticated animals and/or pets.

Techniques for measuring the number of isolated TAF MSCs are known to the skilled person. For example, a cell counter, such as the Nucleocounter 202 (NC-202) may be used to measure the number of isolated TAF MSCs in a sample. From this measurement, the cellular concentration can be adjusted. This adjustment may take place prior to, during, and/or after use of the isolated TAF MSCs in a method, therapeutic use and/or system.

In some embodiments, the use, method or system according to any preceding claim, further comprising an anticoagulant. Suitable anticoagulants are known to the skilled person. For example, the anticoagulant may be selected from the group consisting of heparin, antithrombin agent (e.g. bivalirudin), factor Xa inhibitors (e.g., rivaroxaban, argatroban), factor XIIa inhibitors, and nafamostat mesylate). Preferably, the anticoagulant is heparin, even more preferably low molecular weight heparin. Accordingly, the uses, methods and/or systems described herein may further comprise an anticoagulant, for example heparin, preferably low molecular weight heparin). Heparin may be used at an initial infusion rate of 7.5-20.0 units/kg/h. Suitable concentration ranges of heparin are known to the skilled person, for example as used in Millar et al., 2019.

In some embodiments, the use, method or system according to any preceding claim, further comprising at least one pharmaceutically acceptable carrier, excipient or further component such as therapeutic and/or prophylactic ingredient. A “pharmaceutically acceptable carrier” as referred to herein, is any known compound or combination of known compounds that are known to those skilled in the art to be useful in formulating pharmaceutical compositions. The carrier may include one or more excipients or diluents.

The anticoagulant (e.g. heparin) may be introduced as a formulation. The anticoagulant (e.g. heparin) may be introduced to the ECMO system simultaneously, sequentially and/or subsequently to the isolated TAF MSCs.

In some embodiments, the isolated TAF MSCs are a clonal population. By “clonal” we include the meaning that the population is generated from a single precursor cell. In some embodiments, the isolated TAF MSCs are a mix of clonal populations. The isolated TAF MSCs may be homogeneous (i.e. of uniform composition or character) or heterogeneous (i.e. not of uniform composition or character).

In some embodiments, the isolated TAF MSCs may be in a single-cell suspension. Alternatively, the isolated TAF MSCs may be pelleted and in need of re-suspension prior to subsequent use. Alternatively, or additionally, the isolated TAF MSCs may be in a frozen state, and therefore require thawing before use.

In some embodiments, the isolated TAF MSCs are capable of forming colony forming units (CFU) in culture. The skilled person is aware of culture conditions (e.g. 2D and 3D culture conditions) that can be used to assess whether MSCs are capable of forming CFU in culture.

In some embodiments, the isolated TAF MSCs are functionally characterised. For example, the isolated TAF MSCs may be characterised based on the release of cytokines implicated in growth stimulation or suppression; and/or differentiation into osteogenic, chondrogenic or adipogenic cell lineages), which indicates their ability to differentiate into bone, cartilage or fat lineages. MSCs may be characterised by plastic adhesion, expression of certain cell surface markers (e.g. receptors), absence of other cell surface markers (e.g. receptors), and/or their ability to differentiate into bone, cartilage and fat lineages. In some embodiments, the TAF MSCs have a likeness to certain tissue type MSCs, e.g. lung. By “likeness” we include the meaning that the TAF MSCs express similar or the same markers to the tissue type MSCs, express similar or the same cytokine profiles, lack expression of similar or the same markers, and/or functionally behave in a similar or the same way.

In some embodiments, the isolated TAF MSCs are functionally characterised based on fewer activated T helper cells, more regulatory T cells, fewer activated cytotoxic T cells, and/or fewer effector T cells following co-culture with PBMCs, in comparison with PBMC controls not treated with TAF MSCs (as shown in Example 2). In some embodiments, the isolated TAF MSCs are functionally characterised based on fewer activated macrophages (e.g. M1 and/or M2 macrophages) following co-culture with PBMCs, in comparison with PBMC controls not treated with TAF MSCs (as shown in Example 2).

In some embodiments, the isolated TAF MSCs have been pre-sorted or enriched to contain markers of interest using the techniques described herein.

In some embodiments, the isolated TAF MSCs have been passaged multiple times. For example, the isolated TAF MSCs may have been passaged 1, 2, 3, 4, 5, 6, or more times.

Mesenchymal stem cells may be obtained from amniotic fluid by a method comprising: providing term amniotic fluid (TAF); removing particulate material from the TAF to obtain purified TAF cells; performing adherence selection on the purified TAF cells to obtain TAF adherence cells; passaging the TAF adherence cells to obtain TAF mesenchymal stem cells (TAF MSCs); and selecting TAF MSCs that express a marker selected from the group consisting of TBC1 domain family member 3K (TBC1D3K), allograft inflammatory factor 1 like (AIF1L), cadherin related family member 1 (CDHR1), sodium/potassium transporting ATPase interacting 4 (NKAIN4), ATP binding cassette subfamily B member 1 (ABCB1), plasmalemma vesicle associated protein (PLVAP), mesothelin (MSLN), L1 cell adhesion molecule (L1CAM), hepatitis A virus cellular receptor 1 (HAVCR1), mal, T cell differentiation protein 2 (gene/pseudogene) (MAL2), SLAM family member 7 (SLAMF7), double C2 domain beta (DOC2B), endothelial cell adhesion molecule (ESAM), gamma-aminobutyric acid type A receptor beta1 subunit (GABRB1), cadherin 16 (CDH16), immunoglobulin superfamily member 3 (IGSF3), desmocollin 3 (DSC3), regulator of hemoglobinization and erythroid cell expansion (RHEX), potassium voltage-gated channel interacting protein 1 (KCNIP1), CD70 molecule (CD70), GDNF family receptor alpha 1 (GFRA1), crumbs cell polarity complex component 3 (CRB3), claudin 1 (CLDN1), novel transcript (AC118754.1), sodium voltage-gated channel alpha subunit 5 (SCN5A), fibroblast growth factor receptor 4 (FGFR4), potassium two pore domain channel subfamily K member 3 (KCNK3), dysferlin (DYSF), ephrin A1 (EFNA1), potassium inwardly rectifying channel subfamily J member 16 (KCNJ16), membrane associated ring-CH-type finger 1 (MARCHF1), synaptotagmin like 1 (SYTL1), calsyntenin 2 (CLSTN2), integrin subunit beta 4 (ITGB4), vesicle associated membrane protein 8 (VAMP8), G protein-coupled receptor class C group 5 member C (GPRC5C), CD24 molecule (CD24), cadherin EGF LAG seven-pass G-type receptor 2 (CELSR2), cadherin 8 (CDH8), glutamate receptor interacting protein 1 (GRIP1), dematin actin binding protein (DMTN), F11 receptor (F11R), cell adhesion molecule 1 (CADM1), cadherin 6 (CDH6), coagulation factor II thrombin receptor like 2 (F2RL2), LY6/PLAUR domain containing 1 (LYPD1), solute carrier family 6 member 6 (SLC6A6), desmoglein 2 (DSG2), adhesion G protein-coupled receptor G1 (ADGRG1), cholecystokinin A receptor (CCKAR), oxytocin receptor (OXTR), integrin subunit alpha 3 (ITGA3), adhesion molecule with Ig like domain 2 (AMIGO2), cadherin EGF LAG seven-pass G-type receptor 1 (CELSR1), EPH receptor B2 (EPHB2).

In another aspect, the isolated TAF MSCs are obtainable by the method according to the present disclosure, said cells expressing a surface marker selected from the group comprising of TBC1 domain family member 3K (TBC1D3K), allograft inflammatory factor 1 like (AIF1L), cadherin related family member 1 (CDHR1), sodium/potassium transporting ATPase interacting 4 (NKAIN4), ATP binding cassette subfamily B member 1 (ABCB1), plasmalemma vesicle associated protein (PLVAP), mesothelin (MSLN), L1 cell adhesion molecule (L1CAM), hepatitis A virus cellular receptor 1 (HAVCR1), mal, T cell differentiation protein 2 (gene/pseudogene) (MAL2), SLAM family member 7 (SLAMF7), double C2 domain beta (DOC2B), endothelial cell adhesion molecule (ESAM), gamma-aminobutyric acid type A receptor beta1 subunit (GABRB1), cadherin 16 (CDH16), immunoglobulin superfamily member 3 (IGSF3), desmocollin 3 (DSC3), regulator of hemoglobinization and erythroid cell expansion (RHEX), potassium voltage-gated channel interacting protein 1 (KCNIP1), CD70 molecule (CD70), GDNF family receptor alpha 1 (GFRA1), crumbs cell polarity complex component 3 (CRB3), claudin 1 (CLDN1), novel transcript (AC118754.1), sodium voltage-gated channel alpha subunit 5 (SCN5A), fibroblast growth factor receptor 4 (FGFR4), potassium two pore domain channel subfamily K member 3 (KCNK3), dysferlin (DYSF), ephrin A1 (EFNA1), potassium inwardly rectifying channel subfamily J member 16 (KCNJ16), membrane associated ring-CH-type finger 1 (MARCHF1), synaptotagmin like 1 (SYTL1), calsyntenin 2 (CLSTN2), integrin subunit beta 4 (ITGB4), vesicle associated membrane protein 8 (VAMP8), G protein-coupled receptor class C group 5 member C (GPRC5C), CD24 molecule (CD24), cadherin EGF LAG seven-pass G-type receptor 2 (CELSR2), cadherin 8 (CDH8), glutamate receptor interacting protein 1 (GRIP1), dematin actin binding protein (DMTN), F11 receptor (F11R), cell adhesion molecule 1 (CADM1), cadherin 6 (CDH6), coagulation factor II thrombin receptor like 2 (F2RL2), LY6/PLAUR domain containing 1 (LYPD1), solute carrier family 6 member 6 (SLC6A6), desmoglein 2 (DSG2), adhesion G protein-coupled receptor G1 (ADGRG1), cholecystokinin A receptor (CCKAR), oxytocin receptor (OXTR), integrin subunit alpha 3 (ITGA3), adhesion molecule with Ig like domain 2 (AMIGO2), cadherin EGF LAG seven-pass G-type receptor 1 (CELSR1), EPH receptor B2 (EPHB2).

Alternatively, or additionally, a method for obtaining TAF MSCs from term amniotic fluid may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF MSCs; and selecting the TAF MSCs from the population as cells that express at least one Group A surface marker selected from the group consisting of TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2, thereby obtaining the TAF MSCs.

In some embodiments, selecting TAF MSCs may comprise selecting TAF MSCs that have a reduced expression of markers selected from the group consisting of IL13RA2, CLU, TMEM119, CEMIP, LSP1, GPNMB, FAP, CRLF1, MME, CLMP, BGN, DDR2. Removing particulate matter may comprise filtering and centrifuging the TAF. Performing adherence selection on the purified TAF cells may comprise adhering the purified TAF cells to a surface coated with a vitronectin-based substrate. The selecting step may be performed using fluorescence activated cell sorting (FACS). The selecting step may be performed with antibodies directed to any of the markers or surface markers. The selecting step may comprise selecting TAF MSCs that express at least two markers from the Group A surface markers. The selecting step may comprise selecting TAF MSCs that express at least three markers from the Group A surface markers. The selecting step may comprise selecting TAF MSCs that express at least four markers from the Group A surface markers. The selecting step may comprise a plurality of sorting steps, each sorting step comprising directing TAF MSCs into a first output group or a second output group in dependence on a set of markers expressed or not expressed by the respective TAF MSCs.

In some embodiments, the selecting step may comprise a first sorting step to direct TAF MSCs that express a Group A surface marker into a first output group, and a second sorting step to direct TAF MSCs from the first output group that express a second set of markers into a second output group.

In certain embodiments, a method for obtaining term amniotic fluid lung mesenchymal stem cells (lung TAF MSCs) from term amniotic fluid, may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the lung TAF MSCs; and selecting the TAF lung MSCs from the population as cells that express at least one Group B surface marker selected from the group consisting of PCDH19, DDR1, MME, IFITM10, BGN, NOTCH3, SULF1, TNFSF18, BDKRB1, FLT1, PDGFRA, TNFSF4, UNC5B, FAP, CASP1, CD248, DDR2, PCDH18, LRRC38, and CRLF1, thereby obtaining lung TAF MSCs.

Selecting lung TAF MSCs may comprise excluding MSCs that express a marker selected from the group consisting of CD24, ITGB4, TNFSF10, GFRA1, CD74, FGFR4, HAVCR1, and OSCAR. The selecting step may comprise selecting TAF MSCs that express at least two surface markers from the Group B surface markers. The selecting step may comprise selecting TAF MSCs that express at least three surface markers from the Group B surface markers. The selecting step may comprise selecting TAF MSCs that express at least four surface markers from the Group B surface markers. The selecting step may comprise selecting TAF MSCs that express a surface marker selected from the group of CD248, DDR1, and LRRC38. The selecting step may comprise selecting TAF MSCs that express CD248. The selecting step may comprise selecting TAF MSCs that express CD248 in combination with a marker selected from the group of DDR1 and LRRC38. The selecting step may comprise selecting TAF MSCs that express CD248, DDR1, and LRRC38. In some examples, isolated TAF MSCs may be obtainable by the methods described above, said cells expressing at least one Group A surface marker.

In some embodiments, an isolated population of TAF MSCs, may express at least one Group A surface marker selected from the group comprising of TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2.

In some embodiments, a composition may comprise the isolated TAF MSCs described above and a pharmaceutically acceptable carrier for the TAF MSCs. Isolated lung TAF MSCs obtainable by a method described above may express at least one Group B surface marker selected from the group consisting of PCDH19, DDR1, MME, IFITM10, BGN, NOTCH3, SULF1, TNFSF18, BDKRB1, FLT1, PDGFRA, TNFSF4, UNC5B, FAP, CASP1, CD248, DDR2, PCDH18 and CRLF1. In certain examples, isolated lung TAF MSCs may express at least one Group B surface marker.

In some embodiments, a method for obtaining term amniotic fluid kidney mesenchymal stem (kidney TAF MSCs) cells from term amniotic fluid, may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF kidney MSCs; and selecting the TAF kidney MSCs from the population as cells that express at least one Group C surface marker selected from the group consisting of HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1, thereby obtaining kidney TAF MSCs.

In certain embodiments, isolated kidney TAF MSCs may express at least one Group C surface marker selected from the group consisting of HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1. A composition may comprise the isolated kidney TAF MSCs as described above.

In some embodiments, a method for obtaining term amniotic fluid skin mesenchymal stem cells (skin TAF MSCs) from term amniotic fluid may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF skin MSCs; and selecting the skin TAF MSCs from the population as cells that express at least one Group D surface marker selected from the group consisting of TNFSF18, PCDH19, NCAM2, TNFSF4, CD248, DDR2, HTR2B, PCDH18, SULF1, MME, ADGRA2, DCSTAMP, PDGFRA, UNC5B, SCUBE3, CEMIP, BDKRB1, FLT1, BDKRB2, FAP, CASP1, and SRPX2; and obtaining skin TAF MSCs.

In certain embodiments, isolated skin TAF MSCs may express at least one Group D surface marker selected from the group consisting of TNFSF18, PCDH19, NCAM2, TNFSF4, CD248, DDR2, HTR2B, PCDH18, SULF1, MME, ADGRA2, DCSTAMP, PDGFRA, UNC5B, SCUBE3, CEMIP, BDKRB1, FLT1, BDKRB2, FAP, CASP1, and SRPX2. A composition may comprise the isolated skin TAF MSCs described above and a pharmaceutically acceptable carrier for the skin TAF MSCs.

In some embodiments, a method for obtaining neural TAF MSCs from term amniotic fluid may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF neural MSCs; and selecting the TAF neural MSCs from the population as cells that express at least one Group E surface marker selected from the group consisting of HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1, and ITGB4; thereby obtaining neural TAF MSCs.

In some embodiments, an isolated population of neural TAF MSCs may express at least one Group E surface marker selected from the group consisting of HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1 and ITGB4. A composition may comprise the isolated population of neural TAF MSCs described above and a pharmaceutically acceptable carrier for the neural TAF MSCs.

Due to the propensity for MSCs to clog in oxygenator membranes, smaller MSCs are more advantageous for use in ECMO. In some embodiments, the cell population used is MSCs. In some embodiments, the cell population used is characterised as “small MSCs”, wherein the size of the MSCs is between 15-25 μm average diameter, preferably between 18-22 μm average diameter. In some embodiments, the cell population used is amniotic fluid MSCs. In some embodiments, the cell population used is characterised as “small amniotic fluid MSCs”, wherein the size of the amniotic fluid MSCs is between 15-25 μm average diameter, preferably between 18-22 μm average diameter. In some embodiments, the cell population is lung selected MSCs. In some embodiments, the cell population is lung selected small MSCs, wherein the cells are characterised as small by having an average diameter of between 15-25 μm, preferably between 18-22 μm. In some embodiments, the cell population is lung selected amniotic fluid MSCs. In some embodiments, the cell population is lung selected small amniotic fluid MSCs, wherein the cells are characterised as small by having an average diameter of between 15-25 μm, preferably between 18-22 μm. For all embodiments described herein, the isolated TAF MSCs may be replaced by any of these cell populations. In a particularly preferred embodiment, the size of the isolated TAF MSCs does not exceed 22 μm. For example, in some embodiments, at least 70%, 80%, 90%, 95% or more of the total population of TAF MSCs are 25 μm or 22 μm diameter.

In some embodiments, the isolated TAF MSCs are between 15-25 μm diameter. In a preferred embodiment, the isolated TAF MSCs are between 18-22 μm diameter. By “between”, we intend to include the diameters specified at either end of a range. For example, “between 15-25 μm” may include isolated TAF MSCs that have a diameter of 15, 16, 17, 18, 19, 20, 21, 22, 23, 24 and/or 25 μm. In some embodiments, only a portion of the population are present between the aforementioned diameters. For example, in some embodiments, at least 70%, 80%, 90%, 95% or more are between 15-25 μm or 18-22 μm diameter. Alternatively, or additionally, at least 70%, 80%, 90%, 95% or more have a diameter that is more than 15, 16, 17, 18, 19, 20, 21, 22, 23 or 24 μm in diameter. Alternatively, or additionally, at least 70%, 80%, 90%, 95% or more have a diameter that is less than 25, 24, 23, 22, 21, 20, 19, 18, 17, 16 μm diameter. For example, in some embodiments, at least 70%, 80%, 90%, 95% or more of the total population of TAF MSCs are 25 μm or 22 μm diameter. For any of the aforementioned values or ranges thereof, it may be that the population of isolated TAF MSCs has an average diameter of the value or within the range. In some embodiments, the size or average size is determined by using a cell counter, such as the Nucleocounter 202 (NucleoCounter® NC-202™, Automated cell counter, chemometec).

In some embodiments, the isolated TAF MSCs comprise lower actin expression and/or fewer vesicles at the surface compared with adult MSCs. Suitable techniques for determining actin and/or vesicle levels are known to the skilled person, such as that described in Mo et al., 2017.

In a preferred embodiment, the isolated TAF MSCs or composition comprising isolated TAF MSCs is formed of an effective amount of lung TAF MSCs. In some embodiments, an effective population of lung TAF MSCs is a population that comprises at least 10%, 20%, 30%, 40%, 50%, 60%, 70%, 80%, 90%, 95%, 96%, 97%, 98% or 99% lung TAF MSCs, wherein the remaining proportion is unsorted TAF MSCs and/or other types of sorted TAF MSCs. Percentage is calculated based on the total number of TAF MSCs. In an embodiment 100% of the population of TAF MSCs are Lung TAF MSCs. The percentage may be an integer between any of the specified values. For example, a population may comprise at least 24% lung TAF MSCs, wherein the remaining percentage is a different type of TAF MSCs (such as unsorted TAF MSCs). As a further example, a mixed population may comprise at least 80% of lung TAF MSCs, wherein the remaining percentage is a different type of TAF MSCs (e.g. unsorted TAF MSCs). The percentage of a particular type of TAF MSCs may relate to any one or more of the markers described herein. For example, at least 24% lung TAF MSCs includes the meaning that, following MSC sorting, at least 24% of the cell population express CD248.

The cell may be “autologous” or “allogeneic”, as described further below.

By “autologous” we include the meaning that the TAF MSCs are derived from cells which originate from an individual to whom the TAF MSCs are to be used in accordance with the various aspects of the present application.

By “allogeneic” we include the meaning that the TAF MSCs are derived from cells which do not originate from the individual to whom the TAF MSCs are to be used in accordance with the various aspects of the present application. Typically, the cells are derived from cells of the same species as the individual on which the methods or uses are to be carried out.

By “composition” we include “pharmaceutical composition”. The phrases “pharmaceutically or veterinarially acceptable” include reference to compositions that do not produce an adverse, allergic or other untoward reaction when administered to an animal or a human, as appropriate. The preparation of such pharmaceutical or veterinary compositions are known to those of skill in the art in light of the present disclosure, as exemplified by Remington's Pharmaceutical Sciences, 18th Ed. Mack Printing Company, 1990, incorporated herein by reference. Moreover, for animal or human administration, it will be understood that preparations should meet sterility, pyrogenicity, general safety and purity standards as required by FDA Office of Biological Standards.

As used herein, “pharmaceutically or veterinarially acceptable carrier” includes any and all solvents, dispersion media, coatings, surfactants, antioxidants, preservatives (e.g., antibacterial agents, antifungal agents), isotonic agents, salts, preservatives, drugs, drug stabilizers, excipients, disintegration agents, such like materials and combinations thereof, as would be known to one of ordinary skill in medicine. Except insofar as any conventional carrier is incompatible with the active ingredient, its use in the therapeutic or pharmaceutical compositions is contemplated.

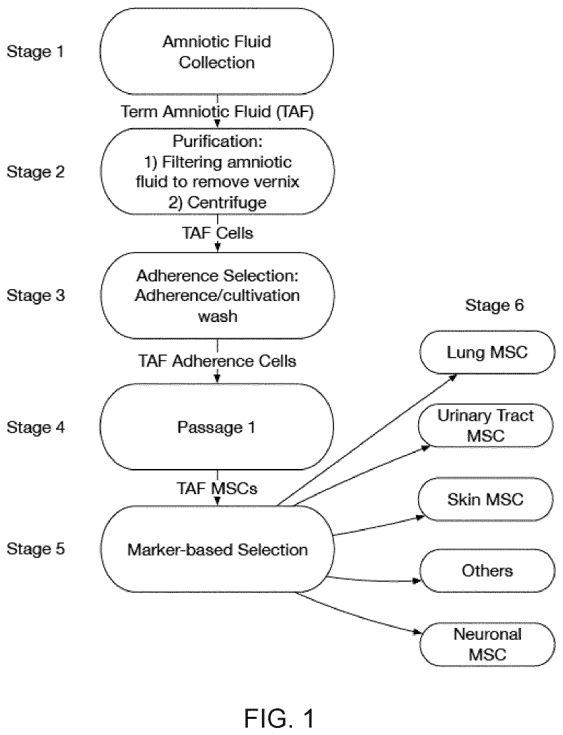

Methods of purifying, culturing and selecting MSC subpopulations with neonatal quality and adult tissue specificity are summarized in FIG. 1 and described in detail below. Examples disclosed herein relate to apparatuses and methods for collecting, purifying, isolating, expanding, differentiating, and maturing amniotic fluid-derived cells. The examples disclosed herein are not limited to collection of a certain type of amniotic-derived cell and the technologies disclosed herein are broadly applicable to different cells and tissues.

### Amniotic Fluid Collection

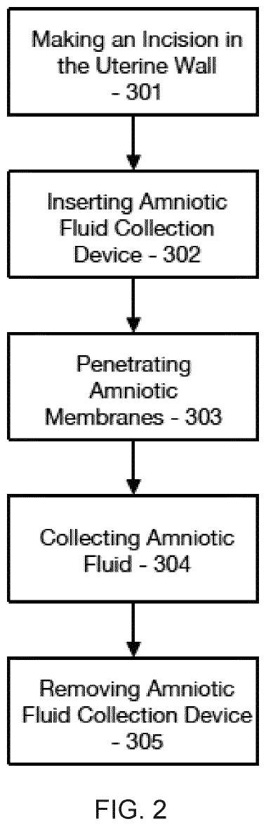

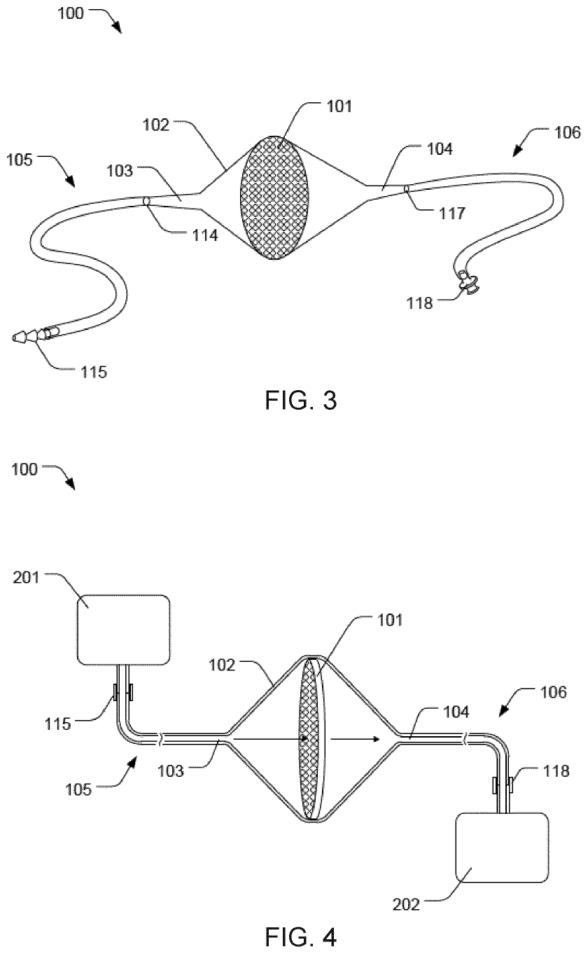

Amniotic fluid may be collected to produce term amniotic fluid (TAF) according to the methods described in U.S. patent application Ser. No. 14/776,499 (corresponding to US2016/0030489), the entire content of which is incorporated by reference. Specifically, FIG. 2 is a block diagram of an example of a method 300 of amniotic fluid collection, according to an exemplary example of the invention. It should be appreciated that method 300 may include any number of additional or alternative tasks. The tasks shown in FIG. 3 need not be performed in the illustrated order, and method 300 may be incorporated into a more comprehensive procedure or process having additional functionality not described in detail herein.

As shown in FIG. 2, method 300 may include making an incision in the uterine wall 301 of a pregnant mother, for example, during caesarean section. Step 301 may be performed with a standard physician's scalpel. As also shown in FIG. 2, method 300 may include inserting an amniotic fluid collector 302 through the incision in the uterine wall made in Step 301. Method 300 also includes penetrating the amniotic membrane 303 using the amniotic fluid collector of Step 302. Step 303 may also include penetrating the chorionic membrane. In one aspect, the tip is inserted to a 10 cm depth. In some examples, the tip is inserted to a depth of about 3 cm to about 30 cm. In some examples, the tip is inserted to a depth of about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, or about 29 cm.

Method 300 further includes collecting the amniotic fluid 304 from the amniotic sac using the amniotic fluid collector of Step 302. Step 304 may include initiating a siphon to transfer the amniotic fluid to a collection chamber of the amniotic fluid collector, such as by opening an inlet valve of the amniotic fluid collector. Step 304 may also include positioning a collection chamber of the amniotic fluid collector below an inlet of the amniotic fluid collector. Step 304 may also include coupling a negative pressure source to an outlet of the amniotic fluid collector to initiate transfer of the amniotic fluid. Step 304 may include relocating an inlet of the amniotic fluid collector to retrieve substantially all of the available amniotic fluid.

Finally, method 300 includes removing the amniotic fluid collector 305 from the amniotic sac. Step 305 may include closing an inlet valve of the amniotic fluid collector. In one example, no blood is visible in the collected material. Step 305 may also include emptying the collection system for further use/processing and sterilizing the exterior of the entire device. In one example, the exterior is sterilized using 70% ethanol so that the sterility may be maintained in any post-processing steps, such as in a laminar air flow bench setup, e.g., for isolation of cell material according to the present invention, and for fluid storage.

In one example, the amniotic fluid collection procedure is performed in less than one minute. In one example, the amniotic fluid collection procedure is performed in one to two minutes. In one example, the amniotic fluid collection procedure is performed in not more than three minutes. In one example, the method is simplified compared to standard operating procedures for cesarean sections, for example, by preventing spillage of the amniotic fluid into the operating wound, improving visibility and physical access. In one example, fetal skin is unaffected by the device tip.

### Purification

Term amniotic fluid (TAF) is purified by filtering term amniotic fluid to remove vernix. Although the term ‘term amniotic fluid’ is employed here and elsewhere in the present disclosure, it is understood that methods, processes, and devices of the present disclosure may be applied to all amniotic fluids and not just term amniotic fluid. Term amniotic fluid may be amniotic fluid collected at term caesarean section deliveries using, for example, a closed catheter-based system. For the purposes of the present description, ‘term amniotic fluid’ may be amniotic fluid collected at planned cesarean sections after 37 completed weeks of pregnancy or later, or at planned cesarean section close to term, for example after 36 completed weeks of pregnancy. Preferably, term amniotic fluid is taken at planned caesarean sections during week 37 of pregnancy or later.

FIG. 3 is a schematic illustration of an apparatus 100 for filtering amniotic fluid according to one example. The amniotic fluid contains amniotic cells originating from the fetus or the amniotic sac such as Mesenchymal stem cells. The amniotic fluid also contains other materials chafed off the skin such as hair and vernix. Material other than the amniotic cells are here referred to as particulate matter and may also comprise meconium, blood clots, etc. Particulate matter may be considered as anything larger than 20 μm. For the purposes of filtering, it may be particularly advantageous to treat anything larger than 30 μm or even 50 μm as particulate matter. Optionally, anything larger than the targeted amniotic cells may be treated as particulate matter. The amniotic fluid thus generally contains a mixture of amniotic cells and particulate matter. The apparatus 100 comprises a filter 101 for filtering the particulate matter from the amniotic fluid, and a chamber 102 enclosing the filter 101. The chamber 102 comprises a fluid inlet 103 and a fluid outlet 104. The chamber 102 enclosing the filter 101 should be construed as the filter 101 being isolated by the chamber towards the environment surrounding the chamber 102 such that there is no fluid communication between the amniotic fluid in the chamber 102 with said environment. Fluid communication through the chamber 102 is thus controlled via the fluid inlet 103 and the fluid outlet 104 in the example of FIG. 3. The filter 101 is attached to the inside of the chamber 102 between the fluid inlet 103 and the fluid outlet 104. FIG. 12 shows an example of a cross-section A-A as indicated in FIG. 12 of a circular chamber 102 and filter 101. It should however be understood that the chamber 102 and filter 101 may have varying shapes for optimization to different applications. The apparatus 100 comprises an inlet connector 105 arranged to form a sealing connection between the fluid inlet 103 and an amniotic fluid sample source 201 (shown in FIG. 4). FIG. 4 shows a schematic example of such source 201 of amniotic fluid. Having an inlet connector 105 connected to the fluid inlet 103 and configured to provide a sealing connection between the fluid inlet 103 directly to a source 201 of amniotic fluid provides for minimizing exposure to contaminants and an efficient aseptic handling of the amniotic fluid. This facilitates obtaining amniotic cells which allows post-filtration processing at an improved quality standard. Hence, an aseptic pharmaceutical production process is facilitated. The preparation of e.g. surfactant molecules may be facilitated. The apparatus 100 provides for improving the functioning of the amniotic stem cells, such as an improved engraftment phase following transplantation. Such improved processes are enabled by having the filter 101 enclosed in a chamber 102 and an inlet connector 105 arranged to form a sealing connection between the fluid inlet 103 of the chamber 102 and an amniotic fluid sample source 201. The risk of exposing the amniotic stem cells to contaminants, such as bacteria and viruses, is thus reduced. Exposure to oxygen is also minimized, which provides for reducing formation of oxygen free radicals which may negatively impact the functioning of the stem cells.

FIG. 3 shows an example where the inlet connector 105 comprises a tube 105 connected to the fluid inlet 103 at a first sealing connection 114. The inlet connector 105 may form a sealing connection with the fluid inlet 103 with a force-fitting connection, an adhesive, a clamp, or other fixation elements. In another example, such as schematically shown in FIG. 4, the inlet connector 105 is a continuous extension of the fluid inlet 103, without a separate fixation element, e.g. by being formed as a single piece by molding or other material forming techniques. FIGS. 3 and 4 show a second connector 115 configured to form a sealing connection with a sample source 201, such as a container or bag 201 containing amniotic fluid. The second connector 115 may comprise releasable force-fitting connection, a clamp, or a combination thereof, or other releasable fixation elements. The chamber 102, filter 101, fluid inlet 103, fluid outlet 104, and inlet connector 105 may be provided as a kit in a sterile packaging, e.g. as a disposable kit. Such kit, i.e. apparatus 100, thus provides for a facilitated and improved process of filtering and obtaining amniotic stem cells. Hence, in use, the amniotic fluid passes the filter 101 when flowing from the fluid inlet 103 to the fluid outlet 104. The particulate matter is thus deposited on the filter 101 and the amniotic fluid containing the amniotic cells flows through the fluid outlet 104. As seen in the example in FIG. 12, the filter 101 may be connected around its periphery 116 to the inner wall 113 of the chamber 102. This avoids passing of amniotic fluid from the inlet 103 to the outlet 104 without being filtered. The filter 101 may be tensioned or otherwise supported so that a folding or curving of the filter 101 in the chamber 102 is avoided. This maintains a defined mesh or pore size across the area of the filter 101 and thus defined filtering characteristics. Maintaining a defined mesh or pore size also reduces the risk of clogging the filter 101. Long-term performance may accordingly be improved.

The apparatus 100 may comprise an outlet 5 connector 106 to form a sealing connection between the outlet and an amniotic cell-receiving device 202, such as a centrifuge or other amniotic cell-processing equipment downstream of the apparatus 100. FIG. 4 shows a schematic example of such device 202. This minimizes exposure to contaminants and allows efficient aseptic handling of the amniotic fluid in post-filtering processing steps. FIG. 3 shows an example where the outlet connector 106 comprises a tube 106 connected to the fluid outlet 104 at a first sealing connection 117. The outlet connector 106 may form a sealing connection with the fluid outlet 104 with a force-fitting connection, an adhesive, a clamp, or other fixation elements. In another example, such as schematically shown in FIG. 4, the outlet connector 106 is a continuous extension of the fluid outlet 104, without a separate fixation element, e.g. by being formed as a single piece by molding or other material forming techniques. FIGS. 3 and 4 show a second connector 118 configured to form a sealing connection with an amniotic cell-processing device downstream of the apparatus 100, such as a centrifuge 202. The second connector 118 may comprise a force-fitting connection, a clamp, a combination thereof, or other releasable fixation elements. The connection between the second connector 118 and e.g. a centrifuge 202 may thus be repeatedly connected and disconnected, and also re-sealable to maintain a sealing connection in such procedure. The chamber 102, filter 101, fluid inlet 103, fluid outlet 104, inlet connector 105, and outlet connector 106 may be provided as a kit in a sterile packaging, e.g. as a disposable kit. Such kit, i.e. apparatus 100, thus provides a facilitated and improved process of filtering and processing of amniotic stem cells. The apparatus 100 may comprise a pump 122, 123, arranged to pressurize the amniotic fluid to flow from the fluid inlet 103 to the fluid outlet 104. This provides for a more effective filtering of the amniotic fluid. Larger volumes may be filtered in less time.

FIG. 6 shows an example where a pump 122 is connected to the fluid outlet 104 to draw amniotic fluid through the filter 101 in the direction of the indicated arrows. The pump 122 may be arranged at the fluid inlet 103 to push the amniotic fluid through the filter 101. The pump 122 may be a compact manually operated pump integrated with the fluid inlet 103, fluid outlet 104, inlet connector 105, or outlet connector 106.

FIG. 7 shows another example, described in more detail below, where a pump 123 is arranged to pressurize the amniotic fluid to flow from the fluid inlet 103 to the fluid outlet 104. The chamber 102 may comprise a conduit 119 arranged between the fluid inlet 103 and the fluid outlet 104. The pressure in the chamber 102 may be variable in response to fluid and/or gaseous communication through the conduit 119. The flow of amniotic fluid through the filter 101 may thus be optimized depending on the application, e.g. the flow rate through the filter 101 may be increased or decreased by varying the pressure in the chamber 102 via conduit 119.

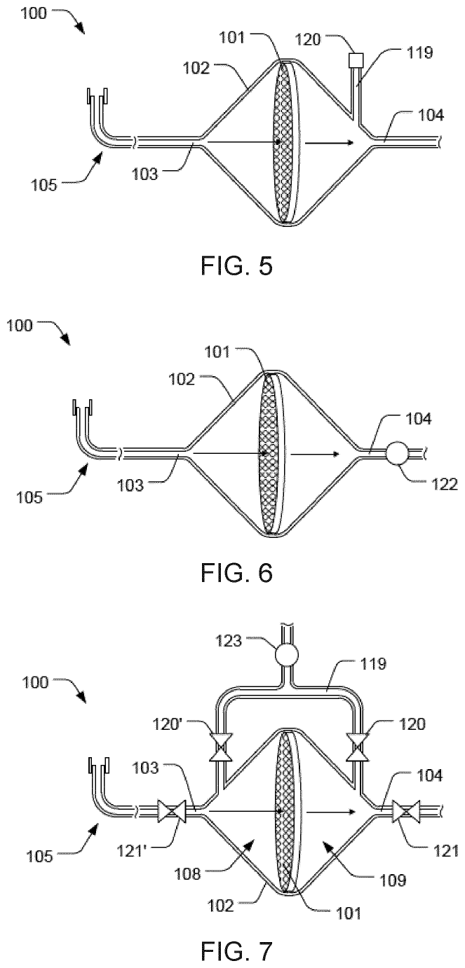

FIG. 5 shows an example in which a conduit 119 is in communication with the chamber 102. An access port 120, such as a connector or valve element, may be actuated to allow a fluid or gas to be expelled from the chamber 102, and/or injected into the chamber 102, to affect the pressure therein. The conduit 119 is arranged between the fluid outlet 103 and the filter 101 in FIG. 5, but the conduit 119 may be arranged between the fluid inlet 103 and the filter 101 in another example. FIG. 5 as described below shows a further example of a conduit 119 in communication with the chamber 102. A pump 123 may be arranged in communication with the conduit 119, as exemplified in FIG. 7. This facilitates optimization of the flow in the chamber 102 and the associated filtering process. In the example of FIG. 7 the conduit 119 is in variable communication with an upstream cavity 108 of the chamber 102 and a downstream cavity 109 of the chamber 102, i.e. the filter 101 may be arranged to divide the chamber 102 into an upstream cavity 108 and a downstream cavity 109. In FIG. 7 the conduit 119 is connected to both the upstream cavity 108 and the downstream cavity 109. The pump 123 is arranged to pressurize the amniotic fluid to flow from the upstream cavity 108 to the downstream cavity 109, or to flow from the downstream cavity 109 to the upstream cavity 108. The latter case may be advantageous in a situation in which a momentary reversed flow is desired, e.g. to clear out clogging or occlusion of the filter 101. In such case, valves 120, 120′, 121, 121′, as schematically indicated in FIG. 7 are operated to provide the desired flow directions. E.g. for a reversed flow, valves 120 and 121′ may be open and valves 120′ and 121 may be closed. Valves 121, 121′, may be open and valves 120, 120′, may be closed in a normal filtering mode. The upstream cavity 108 may be pressurized by also opening valve 120′ in such filtering mode.

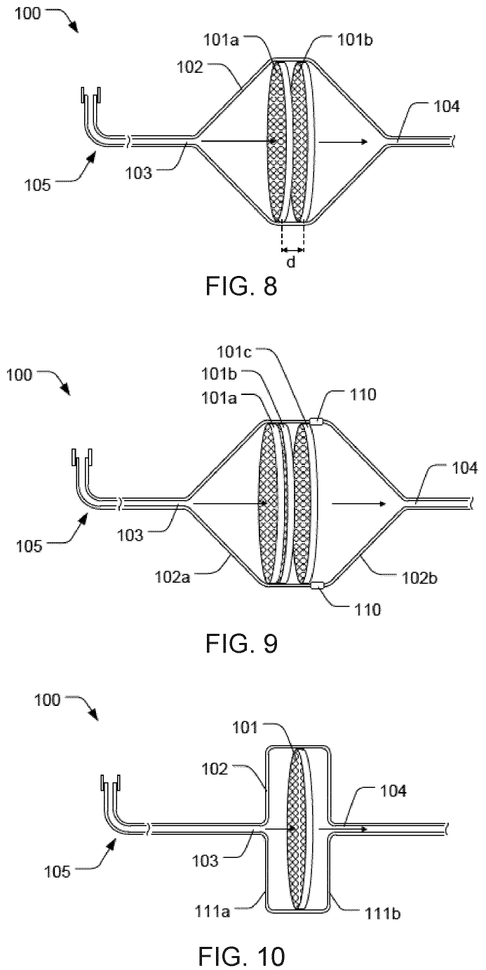

The filter 101 may comprise a first filter element 101a and a second filter element 101b arranged between the first filter element 101a and the fluid outlet 104, as schematically shown in FIG. 8. The second filter element 101b may have a mesh or pore size which is smaller than a mesh or pore size of the first filter element 101a. This allows effective filtering of particulate matter of gradually smaller dimensions. The risk of filter occlusion is thus reduced. This allows for a more reliable and robust filtering process of the amniotic fluid. An improved filtering of amniotic fluid containing a greater range in the size of particulate matter is also provided. Further, a larger fraction of the stem cells in the amniotic fluid may be obtained since the stem cells are not lost in clogged pores. Although FIG. 8 two filter elements 101a, 101b, it should be understood that any plurality of filter elements may be arranged in sequence in the chamber 102, with gradually decreasing mesh or pore size, in the direction of fluid flow from the fluid inlet 103 to the fluid outlet 104, for an effective filtering of particulate matter of gradually decreasing dimensions. The first and second filter elements 101a, 101b, may be separated by a distance (d) along a direction amniotic fluid flow from the fluid inlet 103 to the fluid outlet 104, as schematically indicted in the example of FIG. 8. The motion of the amniotic fluid between the first and second filter elements 101a, 101b, which in some case may involve turbid flow, may provide for further reducing the risk of unwanted build-up of particles on the first and second filter elements 101a, 101b.

The filter 101 may comprise a mesh having a mesh size in the range of 20-2000 μm. In another example, the filter 101 comprises a mesh having a mesh size in the range of 100-500 μm. This allows particularly effective filtration of particulate matter from the amniotic fluid. Turning again to FIG. 8, the first filter element 101a may comprise a mesh having a mesh size in the range of 500-1000 μm, and the second filter element 101b may comprise a mesh having a mesh size in the range of 30-150 μm. The first filter element 101a may thus remove larger debris, followed by removal of smaller particles with the second filter element 101b. This allows a particularly effective filtering of particulate matter of varying size and reliable filtering of increased volumes over longer time periods since the risk of clogging is further minimized. As previously mentioned, any plurality of filter elements may be arranged in succession in the chamber 102.

FIG. 9 shows three filter elements 101a, 101b, 101c, arranged in the chamber 102. In some examples the filter element having the smallest mesh or pore size, arranged furthest downstream in the chamber 102 may, such as filter element 101b in FIG. 6 and filter element 101c in FIG. 9, may have a mesh or pore size dimensioned so that only single amniotic cells or amniotic cell clumps smaller than 10 cells pass through the filter 101. The smallest mesh or pore size in such an example may be approximately 30 μm. The filter 101 may comprise a mesh such as a nylon mesh. The filter 101 may comprise a porous material having a variable pore size through the filter 101 in the direction of flow of the amniotic fluid from the fluid inlet 103 to the fluid outlet 104. I.e. larger debris is removed at the surface of the filter 101 closest to the inlet 103 whereas particles of smaller size are removed deeper into the filter, as the amniotic fluid flows through the filter 101 in a direction towards the outlet 104 and the size of the pores get smaller. As previously mentioned, the chamber 102 may comprise an upstream cavity 108 and a downstream cavity 109. The upstream and downstream cavities 108, 109, may be formed as an integrated piece to form the chamber 102, e.g. in a molding process or by other material forming techniques. The upstream and downstream cavities 108, 109, may be formed as separate units which are then connected to each other to form a sealing connection, e.g. by an adhesive or by welding. The filter 101 may be attached simultaneously or subsequently with such welding process or by the aforementioned adhesive.

The upstream and downstream cavities 108, 109, may be releasably connectable to each other at a connecting element 110, to form a sealing connection, as schematically shown in FIG. 9. This allows opening of the chamber 102, e.g. for replacing the filter 101. The filter 101 may thus be releasably connectable to the chamber 102, e.g. filter elements 101a, 101b, 101c, may be releasably connectable to the chamber 102 in FIG. 7. This allows facilitated customization to different applications since filter elements 101a, 101b, 101c, of different pore or mesh size, or different number of such filter elements may be mounted in the chamber 102.

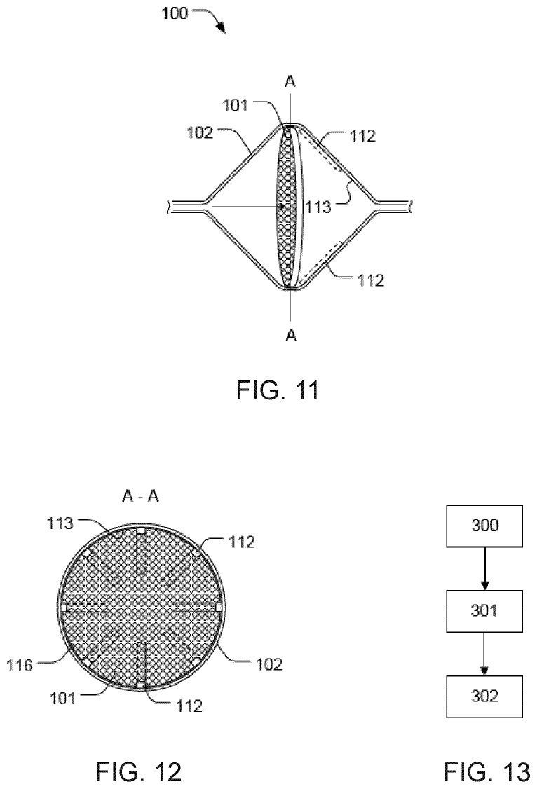

The connecting element 110 is configured to form a sealing connection upstream and downstream cavities 108, 109, and may comprise an annular gasket extending around the periphery of the upstream and downstream cavities 108, 109. The filter 101 may comprise a cartridge of different numbers of filter elements 101a, 101b, 101c, with different pore sizes that could be tailored to the particular amniotic fluid sample. For example, evaluation of the amniotic fluid turbidity and degree of milkiness (level of vernix both in particle size and opaqueness) could be an indicator of the appropriate filter cartridge to use. An accompanying chart for which to compare the amniotic fluid sample with could indicate which filter cartridge to use. The upstream cavity 108 and/or the downstream cavity 109 may be funnel shaped. FIGS. 3-9 show examples where both the upstream and downstream cavities 108, 109, are funnel shaped. FIG. 11 shows an example where only the downstream cavity 109 is funnel shaped. Having a funnel shape may be advantageous for directing the flow of amniotic fluid along a desired vector of symmetry through the filter 101 and apparatus 100. The upstream cavity 108 and/or the downstream cavity 109 may comprise a chamber wall 111a, 111b being arranged essentially in parallel with the filter 101, i.e. perpendicular to the direction of flow of the amniotic fluid from the fluid inlet 103 to the fluid outlet 104. FIG. 10 shows an example where chamber walls 111a, 111b, of the upstream and downstream cavities 108, 109 are arranged essentially in parallel with the filter 101. This minimizes the space inside the chamber 102, while maintaining adequate filter area, to minimize the risk of introducing e.g. air that may disturb surfactant molecules, reduce the risk of infection, and reduce detrimental formation of reactive oxygen species in the amniotic cells. The chamber 102, and/or the inlet connector 105, and/or the outlet connector 106 may be formed from a phthalate free PVC material. This provides for an apparatus which is suitable to be in contact with pharmaceutical starting materials such as amniotic cells.

The apparatus 100 may comprise protrusions 112 arranged to extend from an inner wall 113 of the chamber 102. FIGS. 11 and 12 show examples of such protrusions 112, in a cross-sectional side view and through cross-section A-A respectively. The protrusions 112 provides support for the filter 101 in case the filter 101 would start bend and fold towards the inner wall 113. Thus, a flow through the mesh or pores of the filter 101 is still possible in such case since the filter 101 may be supported by the protrusions 112 at a distance from the inner wall 113, i.e. the protrusions 112 allows for further limiting the risk of flow restriction and provides for an efficient, robust and reliable filtering.

FIG. 13 is a flow chart of a method 300 of filtering amniotic fluid containing particulate matter and amniotic cells. The method 300 comprises forming 301 a sealing connection between a fluid inlet 103 of a chamber 102 and an amniotic fluid sample source 201. The method 300 comprises passing 302 the amniotic fluid through a filter 101 enclosed in the chamber 102 by providing a flow of the amniotic fluid from the fluid inlet 103 to a fluid outlet 104 of the chamber 102. Particulate matter is thereby deposited on the filter 101 and the amniotic fluid containing amniotic cells flows through the outlet 104. The method 300 thus provides for the advantageous benefits as described in relation to apparatus 100 and FIGS. 3-12 above. The method 300 provides for effective and sterile filtration of the amniotic fluid to obtain amniotic cell samples of high quality.

In one embodiment, removing particulate material from the TAF to obtain purified TAF cells may be done by applying any known method in the art such as filtration, centrifugation, etc. The TAF may be filtered through a filter having a pore size at or above 20 μm. The filter may be made from any synthetic material including but not limited to cellulose acetate, cellulose nitrate (collodion), polyamide (nylon), polycarbonate, polypropylene and polytetrafluoroethylene (Teflon). In one embodiment removing particulate material is done by applying apparatus 100.

### Adherence Selection

Various terms known to one skilled in the art have been and will be used throughout the specification, for example, the terms “express, expression, and/or expressing” in the context of a cell surface marker are meant to indicate the presence of a particular marker on the surface of a cell, said surface marker having been produced by the cell. Surface marker expression may be used to select between different cell populations, for example, positively selecting for surface marker expression indicates the selection of a cell population that more strongly expresses a particular surface marker as compared to another cell population. Conversely, negatively selecting for cell surface marker expression indicates the selection of a cell population that more weakly expresses a particular surface marker as compared to another cell population.

As explained above and elsewhere in the specifications, TAF contains various progenitor cell types. In certain examples, particular progenitor cell types may be isolated and propagated via adherence selection. For example, a vitronectin substrate, Synthemax (Merck, CORNING®, Synthemax®, II-SC SUBSTRATE, CLS3535-1EA) may be used as a coating to create a more in vivo-like environment for stem cell culture, thereby limiting maturation of the TAF-derived progenitor cells and maintaining plasticity. Synthemax is an animal-component free, synthetic, flexible vitronectin-based peptide substrate for serum or serum-free expansion of human progenitor/stem cells and other adult stem cell types. One of skill in the art will understand that the vitronectin-based peptide substrate may include a portion of a vitronectin protein, such as a particular peptide sequence of vitronectin. Alternatively, intact vitronectin protein may be used. Synthemax vitronectin substrate offers a synthetic, xeno-free alternative to biological coatings and/or feeder cell layers commonly used in cell culture and known in the art. Briefly, standard tissue-culture treated flasks may be coated with about 0.2 mL Synthemax/cm² at 10 μg/mL giving a surface density of 2 μg/cm², and incubated at 37° C. for about 1 h, 1.5 h, 2 h, 4 h, 8 h, or more than 8 h or at room temperature for about 2 h, 1 h, 4 h, 8 h or more than 8 h with surplus solution optionally being removed and replaced. In certain examples, Synthemax may be coated at a surface density of about: 1 to 5 μg/cm², such as 2 μg/cm², 1 to 10 μg/cm², 1.5 to 4 μg/cm², 1 to 3 μg/cm², or about 1.5 to 2.5 μg/cm².

In other embodiments, adherence selection can be performed using a surface coated with, for example, Collagen, Fibronectin. Alternatively, adherence selection can be performed using an uncoated surface comprising a tissue-culture treated plastic.

Cells purified from TAF fluid may be gently re-suspended in prewarmed xeno-free cell culture media, with the cell suspension is then added to the Synthemax-coated flasks. Media may be changed at various times after addition to the flasks, for example, after about: 2 h to 168 h, 12 h to 96 h, 24 h to 72 h, 36 h to 60 h, 42 h to 56 h, or 48 h, and then subsequently changed about: every day, every other day, every third day, every fifth day, once a week, once every two weeks or about less than once every two weeks. Through repeated removal of spent medium, the non-attached cells may be removed, thereby selecting the MSCs by their affinity for attachment to the Synthemax-treated surface. The cells may be cultured for a period of time, such as about, for example, 4 d, 7 d, 10 d, 11 d, 12 d, 13 d, 14 d, 18 d, 21 d, 28 d or longer than 21 d. Optionally, the cells may be cultured under hypoxic conditions: hypoxia priming may alter cell metabolism during expansion, increase resistance to oxidative stress, and thereby improve the engraftment, survival in ischemic microenvironments, and angiogenic potential of transplanted MSCs. After culturing, the PO colonies (Colony forming Units—CFUs) that have formed may be dissociated and pooled. After pooling, the remaining cells may be predominantly non-tissue specific MSCs. In certain examples, the pooled PO cells may be gently re-suspended in pre-warmed xeno-free cell culture media and re-plated on tissue-culture treated flasks without Synthemax for passaging. The pooled cells may be seeded at a seeding density of from between about: 100 to 10000 cells/cm², 500 to 8000 cells/cm², 1000 to 5000 cells/cm², or about 2000 to 4000 cells/cm². The media may be changed about every 1 d, 2 d, 4 d, or more than four days. After a period of time, such as about 2 d, 4 d, 7 d, or more than 7 d, the cells may be dissociated and harvested. Further selective MSC isolation may be achieved as described below.

### Identification of Markers

When comparing the genetic expression profiles of TAF-MSCs and adult-type MSCs derived from adipose tissue or bone marrow by RNAseq, TAF-MSCs tend to express more of some genes present in adult-type MSCs and less of others. Identification of both positive and negative TAF-MSC specific neonatal cell-surface markers can allow for sorting of the MSCs with neonatal quality from those that have differentiated further and are of less importance as progenitor cells using e.g. ligands such as antibodies and aptamers or other selection techniques.

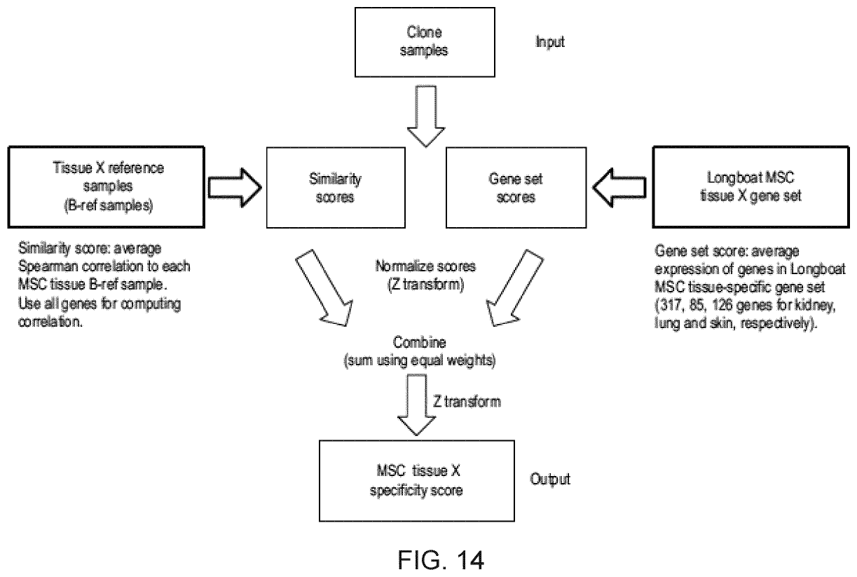

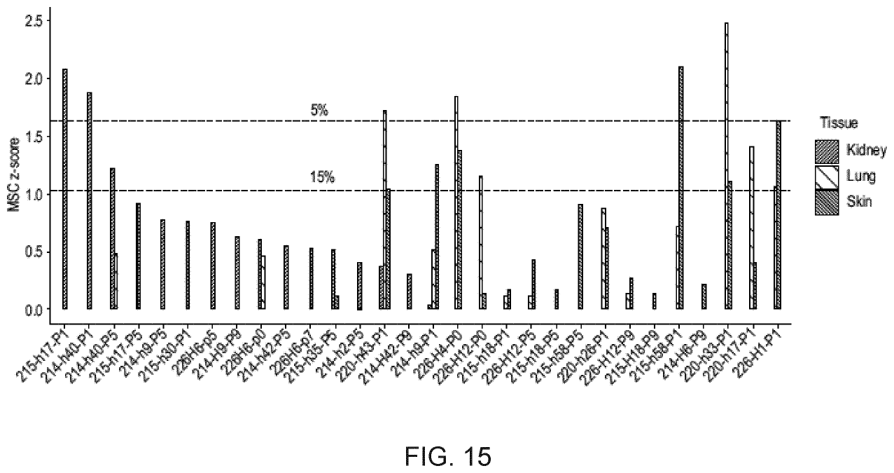

The cell surface markers distinguishing tissue relevant cells from other MSCs may be elucidated via a bioinformatics process utilizing a tissue-specificity score algorithm. An example of an MSC tissue-specificity score algorithm is shown in FIG. 14. Tissue-specificity may be measured as a combination of two components: a ‘tissue transcriptional similarity’ also known as a similarity score and a “tissue-specific gene expression program” also known as a gene set score. In certain examples, the similarity score may be an Average Spearman correlation to each MSC tissue reference sample (for example a fetal lung MSC sample). In examples, the gene set score may be the average expression of genes in a tissue-specific gene set. As shown in FIG. 14, in certain examples, after normalizing the similarity and gene set scores using a Z-transform to convert the input values, which is a sequence of real or complex numbers, into a complex frequency-domain representation, then combining them assigning equal weight to each score and transforming combined values using a Z-transform, the resulting output is an MSC tissue specificity score. The MSC tissue-specificity score measures the relative tissue-specificity among the input samples by measuring how many standard deviations a sample is more or less specific to a given tissue compared to the average input sample. For example, an MSC tissue-specificity score may indicate how much more a clone sample appears to have a tissue specific phenotype, such as a lung phenotype, as compared to an average clone. Such an approach allows for identification of the top X % percentile scores using a normal distribution function, effectively the top X % of clones that are most tissue-specific to the relevant tissue.

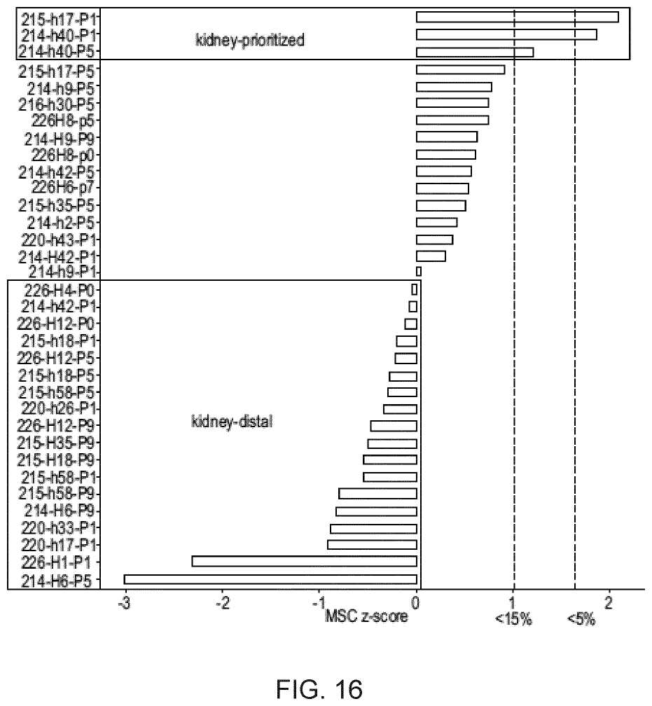

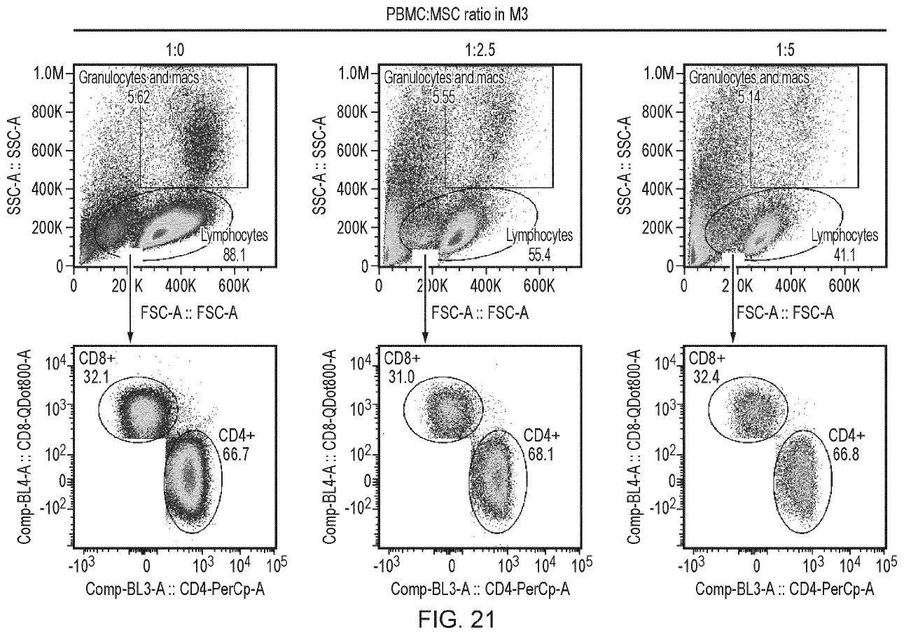

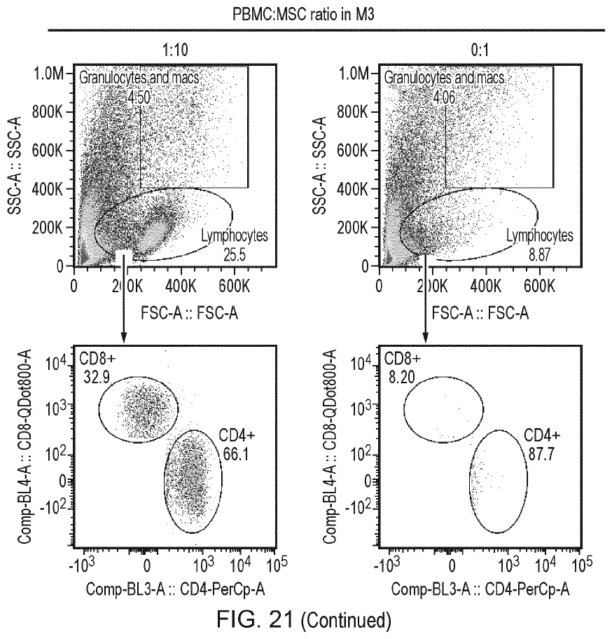

In one example, for a given tissue, tissue-prioritized clones can be defined as any clone belonging to the top X % percentile score, where X is any percentage within a range having a lower end from about 0.1 to 25, such as about 1, 5, 10, and 20, and an upper end from about 30 to 75, such as about: 35, 40, 45, 50, 55, 60, 65 or 70. An example of TAF-MSC tissue-specificity prioritization results is shown in FIG. 21, in which thresholds at 15% and 5% are visible. Having prioritized tissue-specific clones, candidate surface marker genes may then be identified. For each tissue, two groups may be defined: tissue-prioritized and tissue-distal. A suitable analysis program may be used to make this determination, for example DEseq2 from Bioconductor.org. The tissue-prioritized group may include clones with a score in the top 15% percentile. The tissue-distal group may include clones in the bottom Y % percentile in which Y is any percentage within the range having a lower end from about 25 to 70, such as about: 30, 35, 40, 45, 50, 55, 60 or 65 and an upper end from 75 to 99.9, such as about: 80, 85, 90, 95 or 99. FIG. 16 shows an example of such analysis on kidney tissue. Next, differentially expressed genes between the tissue-prioritized and tissue-distal groups may be identified. Finally, the differential expression results may be annotated with surface marker gene information.

In certain examples, to identify tissue-specific cell surface markers, surface marker genes with a more than a Z-fold increase, where Z is at least about: 1.5-fold, 2-fold, 2.5-fold, 3-fold, 3.5-fold, 4-fold, 5-fold, 8-fold, 10-fold, 12-fold, 15-fold or even more-fold increase in expression (log 2FoldChange) in prioritized clones compared to an average clone and a Transcripts Per Kilobase Million (TPM) of more than about 500, such as more than about: 1000, 1500, 2000, 2500, 3000, 5000 or even higher may be selected to give the top tissue-specific marker candidates, such as approximately the top: 5, 10, 20, 30, 40, 50, 60, 70, 100 or more, for example such as those shown below in Tables 3-6 and further described in more detail below. Suitable log 2FoldChange and TPM values may vary even further depending on tissue type specificities depending on the abundance/absence of good markers.

Applying the tissue specificity algorithms described above to identify surface markers, after adhesion selection and passaging, the TAF-MSCs cells may express various identified surface markers as shown below in Table 1, indicative of non-tissue specific TAF MSCs. One of skill in the art will understand that such surface markers may be present at various surface densities and may be upregulated or downregulated in comparison to other cell types. Therefore, such surface markers may be used to identify and isolate particular cell types. In some instances, the surface markers listed in Table 1 below may be at least 8-fold more highly expressed for TAF MSCs on average compared to other MSC cell types, particularly as compared to adult MSCs derived from bone marrow or adipose tissue. The thresholds used to generate Table 1 are as follows: X was selected as 15%, Y was selected as 50%, Z was selected as 8-fold and a TPM of more 3000 was selected. One of skill in the art will understand that the numbering used in Table 1 and all tables herein is merely used to indicate a total number of identified markers and not to indicate that one particular marker is more strongly expressed and/or preferred compared to another marker.

TABLE 1
Group A markers.

| | | |
|---|---|---|
| 1. | TBC1D3K | TBC1 domain family member 3K |
| 2. | AIF1L | allograft inflammatory factor 1 like |
| 3. | CDHR1 | cadherin related family member 1 |
| 4. | NKAIN4 | sodium/potassium transporting ATPase interacting 4 |
| 5. | ABCB1 | ATP binding cassette subfamily B member 1 |
| 6. | PLVAP | plasmalemma vesicle associated protein |
| 7. | MSLN | mesothelin |
| 8. | L1CAM | L1 cell adhesion molecule |
| 9. | HAVCR1 | hepatitis A virus cellular receptor 1 |
| 10. | MAL2 | mal, T cell differentiation protein 2 (gene/pseudogene) |
| 11. | SLAMF7 | SLAM family member 7 |
| 12. | DOC2B | double C2 domain beta |
| 13. | ESAM | endothelial cell adhesion molecule |
| 14. | GABRB1 | gamma-aminobutyric acid type A receptor beta1 |
| | | subunit |
| 15. | CDH16 | cadherin 16 |
| 16. | IGSF3 | immunoglobulin superfamily member 3 |
| 17. | DSC3 | desmocollin 3 |
| 18. | RHEX | regulator of hemoglobinization and erythroid cell |
| | | expansion |
| 19. | KCNIP1 | potassium voltage-gated channel interacting protein 1 |
| 20. | CD70 | CD70 molecule |
| 21. | GFRA1 | GDNF family receptor alpha 1 |
| 22. | CRB3 | crumbs cell polarity complex component 3 |
| 23. | CLDN1 | claudin 1 |
| 24. | AC118754.1 | novel transcript |
| 25. | SCN5A | sodium voltage-gated channel alpha subunit 5 |
| 26. | FGFR4 | fibroblast growth factor receptor 4 |
| 27. | KCNK3 | potassium two pore domain channel subfamily K |
| | | member 3 |
| 28. | DYSF | dysferlin |
| 29. | EFNA1 | ephrin A1 |
| 30. | KCNJ16 | potassium inwardly rectifying channel subfamily J |
| | | member 16 |
| 31. | MARCHF1 | membrane associated ring-CH-type finger 1 |
| 32. | SYTL1 | synaptotagmin like 1 |
| 33. | CLSTN2 | calsyntenin 2 |
| 34. | ITGB4 | integrin subunit beta 4 |
| 35. | VAMP8 | vesicle associated membrane protein 8 |
| 36. | GPRC5C | G protein-coupled receptor class C group 5 member C |
| 37. | CD24 | CD24 molecule |
| 38. | CELSR2 | cadherin EGF LAG seven-pass G-type receptor 2 |
| 39. | CDH8 | cadherin 8 |
| 40. | GRIP1 | glutamate receptor interacting protein 1 |
| 41. | DMTN | dematin actin binding protein |
| 42. | F11R | F11 receptor |
| 43. | CADM1 | cell adhesion molecule 1 |
| 44. | CDH6 | cadherin 6 |
| 45. | F2RL2 | coagulation factor II thrombin receptor like 2 |
| 46. | LYPD1 | LY6/PLAUR domain containing 1 |
| 47. | SLC6A6 | solute carrier family 6 member 6 |
| 48. | DSG2 | desmoglein 2 |
| 49. | ADGRG1 | adhesion G protein-coupled receptor G1 |
| 50. | CCKAR | cholecystokinin A receptor |
| 51. | OXTR | oxytocin receptor |
| 52. | ITGA3 | integrin subunit alpha 3 |
| 53. | AMIGO2 | adhesion molecule with Ig like domain 2 |
| 54. | CELSR1 | cadherin EGF LAG seven-pass G-type receptor 1 |
| 55. | EPHB2 | EPH receptor B2 |

As will be understood by one of skill in the art, suitable combinations of the markers listed in Table 1 may be used to separate TAF-MSCs from adult MSCs by selecting for specific markers from Table 1 or combinations of two, three, four, five, six or more markers from Table 1. In certain examples, TAF MSCs can be more specifically identified by identifying a combination of stronger expression, such as 8-fold or more stronger expression of any combination of the foregoing markers, e.g., TBC1D3K and/or AIF1L and/or CDHR1 and/or NKAIN4 and/or ABCB1 and/or PLVAP as compared to adult MSCs. When using combinations of markers, identification may be achieved with a lower threshold of stronger expression, such as 2-fold or more, 4-fold or more, or 6-fold or more expression of each of the markers.

In contrast to the above surface markers that may be more strongly expressed on the surface of TAF-MSCs (positive markers) compared to adult MSCs, in certain examples, the below surface markers in Table 2 may be more weakly expressed on TAF-MSCs as compared to other cell types (negative markers), such as ⅛-fold or less expression (optionally with TPM threshold >500) of any combination of the foregoing markers versus adult MSCs: IL13RA2, CLU, TMEM119, CEMIP, and LSP1. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate TAF MSCs. In addition, those skilled in the art will also recognize that combinations including both negative and positive markers, such as at any of the thresholds described above, can also be effective to more specifically isolate TAF MSCs.

TABLE 2
Markers that have reduced expression in TAF MSCs.

| | | |
|---|---|---|
| 1. | IL13RA2 | Interleukin-13 receptor subunit alpha-2 |
| 2. | CLU | Clusterin |
| 3. | TMEM119 | Transmembrane Protein 119 |
| 4. | CEMIP | Cell Migration Inducing Hyaluronidase 1 |
| 5. | LSP1 | Lymphocyte Specific Protein 1 |
| 6. | GPNMB | Glycoprotein Nmb |
| 7. | FAP | Fibroblast Activation Protein Alpha |
| 8. | CRLF1 | Cytokine Receptor Like Factor 1 |
| 9. | MME | Membrane Metalloendopeptidase |
| 10. | CLMP | CXADR Like Membrane Protein |
| 11. | BGN | Biglycan |
| 12. | DDR2 | Discoidin Domain Receptor Tyrosine Kinase 2 |

### Marker-Based Selection

Amniotic fluid contains heterogenous cells in a homogenous fluid. Hence, a marker-based selection may be needed. One example of marker-based selection is via the use of Fluorescence activated cell sorting (FACS). Fluorescence activated cell sorting (FACS) may be used to purify the cell population of TAF-MSCs, FACS allows for a very high purity of the desired cell population, even when the target cell type expresses very low levels of identifying markers and/or separation is needed based on differences in marker density. FACS allows the purification of individual cells based on size, granularity and fluorescence. As will be understood by one of skill in the art, FACS may be used to select for certain cell populations that express one cell surface marker more than another cell population and vice-versa. In some examples of methods of purification, bulk methods of purification such as panning, complement depletion and magnetic bead separation, may be used in combination with FACS or as an alternative to FACS. In brief, to purify cells of interest via FACS, they are first stained with fluorescently-tagged monoclonal antibodies (mAbs), which recognize specific surface markers on the desired cell population. Negative selection of unstained cells may also allow for separation. For GMP production of cells according to some examples, FACS may be run using a closed system sorting technology such as MACSQuant® Tyto®. Samples may be kept contamination-free within the disposable, fully closed MACSQuant Tyto Cartridge. Further, filtered air may drive cells through a microchannel into the microchip at very low pressure (<3 PSI). However, before entering the microchannel, potential cell aggregates may be held back by a filter system guaranteeing a smooth sorting process. The fluorescence detection system may detect cells of interest based on predetermined fluorescent parameters of the cells. Based on their fluorescent and scatter light signatures, target cells may be redirected by a sort valve located within the microchannel. For certain examples of methods of purification, the success of staining and thereby sorting may depend largely on the selection of the identifying markers and the choice of mAb. Sorting parameters may be adjusted depending on the requirement of purity and yield. Unlike on conventional droplet sorters, cells sorted by the MACSQuant Tyto may not experience high pressure or charge, and may not get decompressed. Therefore, such a gentle sorting approach may result in high viability and functionality of cells. Alternatively, other marker-based selection techniques may be known to the skilled person and employed here. These include, but are not limited to, Magnetic-activated cell sorting, Microfluidic based sorting, Buoyancy activated cell sorting, mass cytometry etc.

### Tissue Specific Cells and Usage

Luna TAF Cell Markers

As explained above, analysis of RNAseq data from TAF-MSC clones, adult and neonatal MSC reference material as well as fetal fibroblasts and publicly available expression datasets may be used to identify and characterize TAF-MSC cells. For example, sub-populations of TAF-MSCs may be established by clustering their expression data (RNAseq) with neonatal reference samples. Such sub-populations include, but are not limited to, lung MSC, urinary tract MSC (described also as kidney MSCs in the present disclosure), and skin MSC. Gene lists of highly and lowly expressed genes for each cluster of expression data may allow for identification of surface maker genes for each cluster. Using such data comparison, sub-populations of TAF cells were compared to adult MSC cells based on their gene expressions (RNAseq) resulting in a list of neonatal-specific surface marker genes for each cluster. A number of surface markers of interest associated with lung TAF cells were identified. For example, a non-exclusive list of preferred surface markers used to identify and separate lung TAF cells are provided below. Moreover, as the number of different MSC-subtypes in TAF is limited, the selection of the tissue specific MSC may be done by firstly characterization, thereafter a stepwise negative selection/sorting of the material by taking into account the combined (multivariate) surface marker profile of the different tissue specific MSC's. One of skill in the art will understand that any such combination of these surface markers may be used for identifying and isolation of lung TAF cells from the general population of TAF-derived cells and/or TAF-MSC cells. In some examples, the below non-exclusive list of surface markers may be more highly expressed on the surface of Lung-TAF cells as compared to other cell types, such as other TAF-derived cells and/or TAF-MSC cells.

As explained above, bioinformatics techniques may be used to identify tissue-specific surface markers, therefore, the surface markers identified in Table 3 may have at least a 10-fold increase in expression on prioritized clones compared to the average TAF-MSC clone (optionally with TPM threshold >2000).

TABLE 3
Group B markers.

| | | |
|---|---|---|
| 1. | PCDH19 | protocadherin 19; |
| 2. | DDR1 | discoidin domain receptor tyrosine kinase 1 |
| 3. | MME | membrane metalloendopeptidase |
| 4. | IFITM10 | interferon induced transmembrane protein 10; |
| 5. | BGN | biglycan |
| 6. | NOTCH3 | notch receptor 3; |
| 7. | SULF1 | sulfatase 1; |
| 8. | TNFSF18 | TNF superfamily member 18; |
| 9. | BDKRB1 | bradykinin receptor B1; |
| 10. | FLT1 | fms related tyrosine kinase 1 |
| 11. | PDGFRA | platelet derived growth factor receptor alpha; |
| 12. | TNFSF4 | TNF superfamily member 4; |
| 13. | UNC5B | unc-5 netrin receptor B; |
| 14. | FAP | fibroblast activation protein alpha |
| 15. | CASP1 | caspase 1; |
| 16. | CD248 | Endosialin; |
| 17. | DDR2 | discoidin domain receptor tyrosine kinase 2 |
| 18. | PCDH18 | protocadherin 18; and/or |
| 19. | CRLF1 | cytokine receptor like factor 1; |

In contrast to the above surface markers that may be more strongly expressed on the surface of lung TAF MSCs, in certain examples, the below surface markers may be more weakly expressed on lung TAF MSCs as compared to other cell types, such as other TAF-derived cells and/or TAF-MSCs: CD24, ITGB4, TNFSF10, GFRA1, CD74, FGFR4, HAVCR1, and OSCAR. As will be understood by one of skill in the art, one, two, three, four, or more of the aforementioned more weakly expressed surface markers may be used to separate lung TAF cells from other cell types such as other TAF-derived cells and/or TAF-MSCs.

In certain examples, the cell surface marker CD248 (Endosialin) may be used to sort lung TAF MSCs from a population of TAF MSCs. Further surface markers that may be used to sort lung TAF MSCs include DDR-1 (discoidin domain receptor tyrosine kinase 1) as well as LRRC38 (Leucine Rich Repeat Containing Protein 38), all three of which have been identified via antibodies as useful markers for separation. In some examples, Endosialin, DDR-1, and/or LRRC38 alone or in combination with other markers may be used to sort. Endosialin may be combined with DDR-1 or LRRC38 to sort, or DDR-1 and LRRC38 may be combined without Endosialin.

As will be understood by one of skill in the art, suitable combinations of the markers listed in Table 3 and CD248, DDR-1, and LRR38 may be used to separate lung TAF MSCs from TAF MSCs by selecting for specific markers from Table 3 or combinations of two, three, four, five, six or more markers from Table 3 and/or CD248 and/or DDR-1 and/or LRR38. In certain examples, lung TAF MSCs can be more specifically identified by identifying a combination of stronger expression, such as 10-fold or more stronger expression (optionally with TPM threshold >2000) of any combination of the foregoing markers, e.g., PCDH19 and/or DDR1 and/or MME and/or IFITM10 and/or BGN and/or NOTCH3 and/or CD248 and/or DDR-1 and/or LRR38 as compared to TAF MSCs. When using combinations of markers, identification may be achieved with a lower threshold of stronger expression, such as 4-fold or more, 6-fold or more, or 8-fold or more expression of each of the markers.

In contrast to the above surface markers that may be more strongly expressed on the surface of lung TAF MSCs (positive markers) compared to TAF MSCs, in certain examples, the below surface markers may be more weakly expressed on lung TAF-MSCs as compared to other cell types (negative markers), such as ⅛-fold or less expression (optionally with TPM>500) of any combination of the foregoing markers versus TAF MSCs: CD24, ITGB4, TNFSF10, GFRA1, CD74, FGFR4, HAVCR1, and OSCAR. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate lung TAF MSCs. In addition, those skilled in the art will also recognize that combinations including both negative and positive markers, such as at any of the thresholds described above, can also be effective to more specifically isolate lung TAF MSCs.

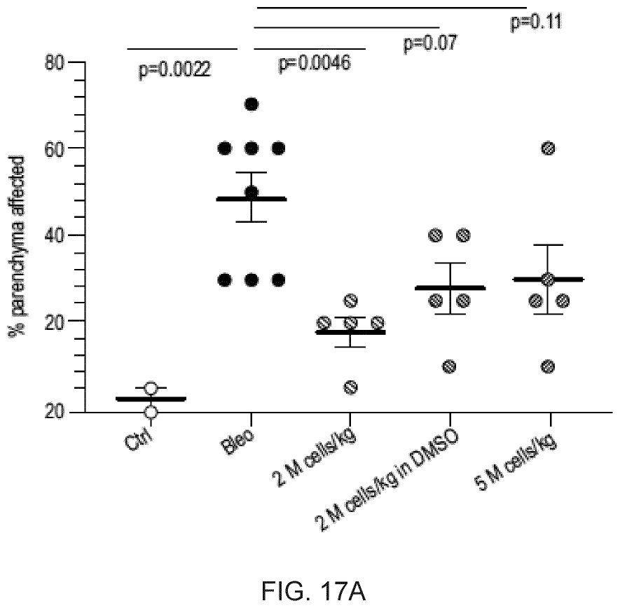

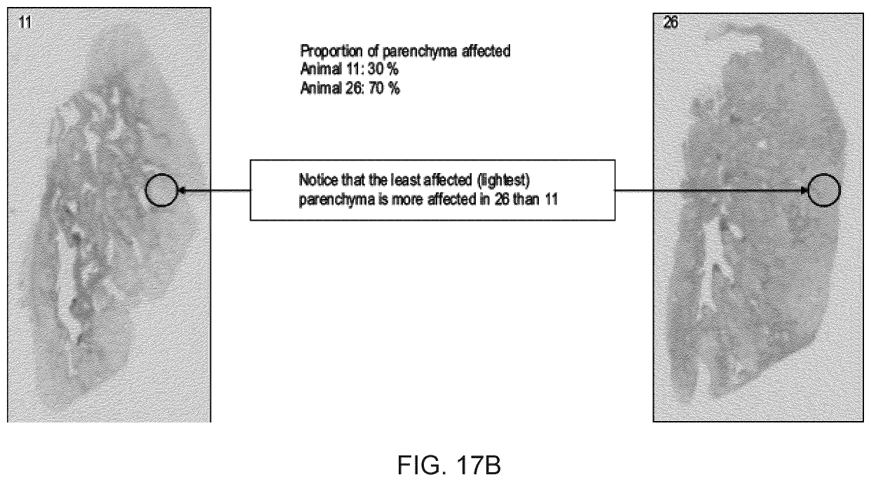

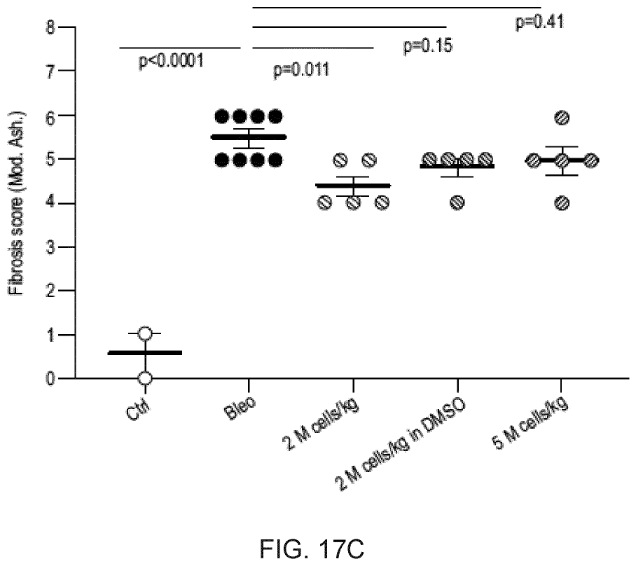

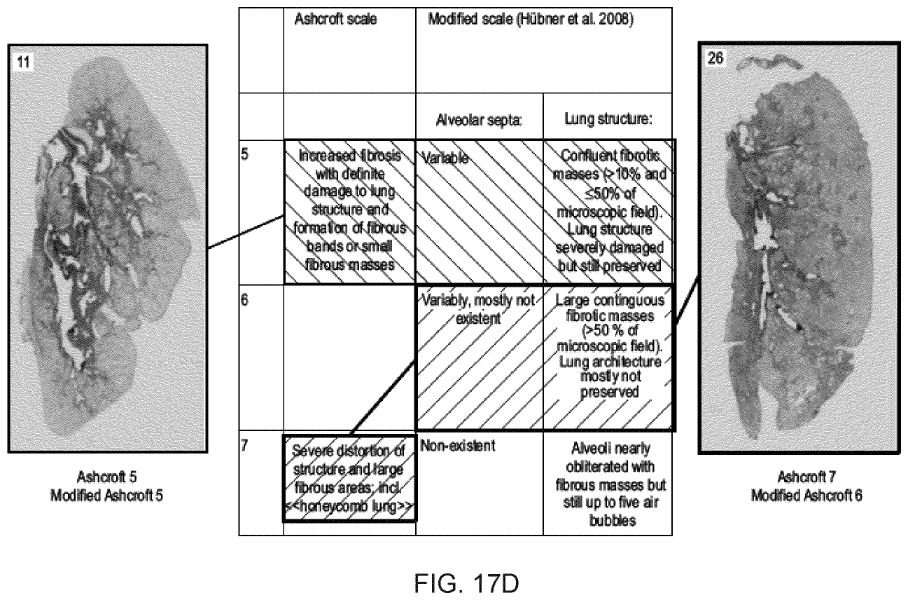

FIGS. 17A-17D show an example of the results from a proof-of-principle study on the potential use of Lung TAF MSCs for treatment, performed using neonatally sorted TAF MSCs expressing MSC lung cell surface markers including CD248, DDR1, and LRRC38 (called “LBX-THX-001 cells”). The purpose of the study was to investigate the effects of LBX-THX-001 cells in a bleomycin induced lung fibrosis model in male rats. Two cell concentrations (2 M cell/kg and 5 M cells/kg) and two types of vehicles for the cells were tested (PBS and CryoStor CS-10).

The development of fibrosis in rat lung after exposure to bleomycin is well documented in the literature and a frequently used model for studying the pathology of lung fibrosis and also the effect of different treatments. The number of LBX-THX-001 cells injected were chosen to be relevant for a possible human therapy. The number of cells were therefore chosen to reflect cell numbers used in previous studies on rats (8-20 M cells/kg) and humans (0.5-2 M cells/kg).

An intra-tracheal instillation of bleomycin (1000 U/rat) to 34 male SD-rats was used to induce lung fibrosis in the rats. During the first week, the rats were monitored and weighed daily and thereafter twice/week until termination of the study. At day 4 post bleomycin challenge, the LBX-THX-001 cells were administered by an intravenous (i.v.) injection. The injection volume was 194-535 μL (maximal tolerated injection volume 1 mL/kg). The response to the intra-tracheal instillation of bleomycin was as expected based on previous experience for the model with weight loss during the first days after instillation and thereafter recovery. There were no significant differences in weight loss between the bleomycin group and the treatment groups.

As shown in FIGS. 17A-D, bleomycin instillation induced fibrotic change in the lung. The histopathological evaluation concluded pathological changes in the bleomycin group both with regard to percent of parenchyma affected and after scoring using the modified Ashcroft scale. As shown in FIGS. 17A-D, the group treated with LBX-THX-001 cells (2 million cells/kg) 4 days post Bleomycin showed significantly less fibrosis in their lungs compared to the bleomycin group. This was seen both in the histopathological evaluation using the read-out “percent parenchyma affected” (FIGS. 17A-B) and the fibrosis scoring Ashcroft modified scale (FIGS. 17A-D). No human MSCs could be detected in rat lungs at termination (after 28 days).

Kidney TAF Cell Markers

Similar to the lung TAF MSC cell markers identified above, a number of surface markers of interest associated with kidney TAF cells were identified. For example, a non-exclusive list of surface markers used to identify and separate kidney TAF MSCs are provided below in Table 4. Similar to the lung TAF MSC markers, the surface markers identified in Table 4 may have at least a 12-fold increase in expression on prioritized kidney TAF clones compared to the average TAF-MSC clone (optionally with TPM threshold >2000). Moreover, as the number of different MSC-subtypes in TAF is limited, the selection of the tissue specific MSCs may be done first by characterization, and thereafter by a stepwise negative selection/sorting of the material by taking into account the combined (multivariate) surface marker profile of the different tissue specific MSCs. One of skill in the art will understand that any such combination of these surface markers may be used for identifying and isolation of kidney TAF cells from the general population of TAF-derived cells and/or TAF-MSC cells. In some examples, the below non-exclusive list of surface markers may be more highly expressed on the surface of kidney-TAF cells as compared to other cell types, such as other TAF-derived cells and/or TAF-MSC cells:

TABLE 4
Group C markers.

| | | |
|---|---|---|
| 1. | HAVCR1 | hepatitis A virus cellular receptor 1; |
| 2. | CD24 | CD24 molecule |
| 3. | CLDN6 | claudin 6; |
| 4. | ABCB1 | ATP binding cassette subfamily B member 1; |
| 5. | SHISA9 | shisa family member 9; |
| 6. | CRB3 | crumbs cell polarity complex component 3 |
| 7. | AC118754.1 | Arachidonate 15-lipoxygenase, ALOX15, |
| | | Smoothelin-like protein 2, SMTNL2, Glutathione |
| | | hydrolase 6, GGT6, Myb-binding protein 1A, |
| | | MYBBP1A, Protein spinster homolog 2, SPNS2 |
| 8. | ITGB6 | integrin subunit beta 6; |
| 9. | CDH1 | cadherin 1 |
| 10. | LSR | lipolysis stimulated lipoprotein receptor |
| 11. | EPCAM | epithelial cell adhesion molecule; |
| 12 | AJAP1 | adherens junctions associated protein 1; |
| 13. | ANO9 | anoctamin 9 |
| 14. | CLDN7 | claudin 7; |
| 15. | EFNA1 | ephrin A1; |
| 16. | MAL2 | mal, T cell differentiation protein 2 (gene/pseudogene) |
| 17. | F11R | F11 receptor |
| 18. | L1CAM | L1 cell adhesion molecule; |
| 19. | GFRA1 | GDNF family receptor alpha 1; |
| 20. | IGSF3 | immunoglobulin superfamily member 3; |
| 21. | TNF | tumor necrosis factor |
| 22. | MMP7 | matrix metallopeptidase 7; |
| 23. | FOLR1 | folate receptor alpha; |
| 24. | TGFA | transforming growth factor alpha |
| 25. | C3 | complement C3 |
| 26. | TNFSF10 | TNF superfamily member 10; |
| 27. | PDGFB | platelet derived growth factor subunit B; and/or |
| 28. | WWC1 | WW and C2 domain containing 1 |

As will be understood by one of skill in the art, suitable combinations of the markers listed in Table 4 may be used to separate kidney TAF cells from TAF-MSCs by selecting for specific markers from Table 4 or combinations of two, three, four, five, six or more markers from Table 4. In certain examples, kidney TAF MSCs can be more specifically identified by identifying a combination of stronger expression, such as 12-fold or more stronger expression (optionally with TPM threshold >2000) of any combination of the foregoing markers, e.g., HAVCR1 and/or CD24 and/or CLDN6 and/or ABCB1 and/or SHISA9 and/or CRB3 as compared to TAF-MSCs. When using combinations of markers, identification may be achieved with a lower threshold of stronger expression, such as 4-fold or more, 6-fold or more, or 8-fold or more expression of each of the markers.

In contrast to the above surface markers that may be more strongly expressed on the surface of kidney TAF MSCs (positive markers), in certain examples, the below surface markers may be more weakly expressed on kidney TAF cells as compared to other cell types (negative markers), such as such as ⅛-fold or less expression (optionally with TPM threshold >500) of any combination of the foregoing markers other TAF-derived cells and/or TAF-MSC cells: GREM1, PDGFRB, BGN, FAP, CXCL12, CCKAR, CD248. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate kidney TAF MSCs. In addition, those skilled in the art will also recognize that combinations including both negative and positive markers, such as at any of the thresholds described above, can also be effective to more specifically isolate kidney TAF MSCs.

Skin TAF Cell Markers

Similar to the lung and kidney TAF MSC markers identified above, a number of surface markers of interest associated with skin TAF cells were identified. For example, a non-exclusive list of surface markers used to identify and separate skin TAF cells are provided below in Table 5. The skin TAF MSC markers identified in Table 5 may have at least a 12-fold increase in expression on prioritized clones compared to the average TAF-MSC clone (optionally with TPM threshold >2000). Moreover, as the number of different MSC-subtypes in TAF is limited, the selection of the tissue specific MSC may be done by firstly characterization, thereafter a stepwise negative selection/sorting of the material by taking into account the combined (multivariate) surface marker profile of the different tissue specific MSC's. One of skill in the art will understand that any such combination of these surface markers may be used for identifying and isolation of skin TAF cells from the general population of TAF-derived cells and/or TAF-MSC cells. In some examples, the below non-exclusive list of surface markers may be more highly expressed on the surface of skin-TAF cells as compared to other cell types, such as other TAF-derived cells and/or TAF-MSC cells:

TABLE 5
Group D markers.

| | | |
|---|---|---|
| 1. | TNFSF18 | TNF superfamily member 18; |
| 2. | PCDH19 | protocadherin 19; |
| 3. | NCAM2 | neural cell adhesion molecule 2; |
| 4. | TNFSF4 | TNF superfamily member 4; |
| 5. | CD248 | Endosialin; |
| 6. | DDR2 | discoidin domain receptor tyrosine kinase 2 |
| 7. | HTR2B | 5-hydroxytryptamine receptor 2B; |
| 8. | PCDH18 | protocadherin 18; |
| 9. | SULF1 | sulfatase 1; |
| 10. | MME | membrane metalloendopeptidase |
| 11. | ADGRA2 | adhesion G protein-coupled receptor A2; |
| 12. | DCSTAMP | dendrocyte expressed seven transmembrane protein; |
| 13. | PDGFRA | platelet derived growth factor receptor alpha; |
| 14. | UNC5B | unc-5 netrin receptor B; |
| 15. | SCUBE3 | signal peptide, CUB domain and EGF like domain |
| | | containing 3; |
| 16. | CEMIP | cell migration inducing hyaluronidase 1; |
| 17. | BDKRB1 | bradykinin receptor B1; |
| 18. | FLT1 | fms related tyrosine kinase 1 |
| 19. | BDKRB2 | bradykinin receptor B2; |
| 20. | FAP | fibroblast activation protein alpha |
| 21. | CASP1 | caspase 1; and/or |
| 22. | SRPX2 | sushi repeat containing protein X-linked 2 |

As will be understood by one of skill in the art, suitable combinations of the markers listed in Table 5 may be used to separate skin TAF MSCs from TAF-MSCs by selecting for specific markers from Table 5 or combinations of two, three, four, five, six or more markers from Table 5. In certain examples, skin TAF MSCs can be more specifically identified by identifying a combination of stronger expression, such as 12-fold or more stronger expression (optionally with TPM >2000) of any combination of the foregoing markers, e.g., TNFSF18 and/or PCDH19 and/or NCAM2 and/or TNFSF4 and/or CD248 and/or DDR2 as compared to TAF-MSCs. When using combinations of markers, identification may be achieved with a lower threshold of stronger expression, such as 4-fold or more, 6-fold or more, or 8-fold or more expression of each of the markers.

In contrast to the above surface markers that may be more strongly expressed on the surface of skin TAF cells (positive markers), in certain examples, the below surface markers may be more weakly expressed on skin TAF cells as compared to other cell types (negative markers), such as such as ⅛-fold or less expression (optionally with TPM threshold >500) of any combination of the foregoing markers other TAF-derived cells and/or TAF-MSC cells: CD24, TNFSF10, ITGB4, ABCB1. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate skin TAF MSCs. In addition, those skilled in the art will also recognize that combinations including both negative and positive markers, such as at any of the thresholds described above, can also be effective to more specifically isolate skin TAF MSCs.

Neural TAF Cell Markers

Similar to the lung, kidney, and skin TAF MSC markers identified above, a number of surface markers of interest associated with neural TAF cells were identified. For example, a non-exclusive list of surface markers used to identify and separate neural TAF cells are provided below. The neural TAF MSC surface markers identified in Table 6 may have at least a 3-fold increase in expression on prioritized clones compared to the average TAF-MSC clone (optionally with TPM threshold >500). Moreover, as the number of different MSC-subtypes in TAF is limited, the selection of the tissue specific MSC may be done by firstly characterization, thereafter a stepwise negative selection/sorting of the material by taking into account the combined (multivariate) surface marker profile of the different tissue specific MSC's. One of skill in the art will understand that any such combination of these surface markers may be used for identifying and isolation of neural TAF cells from the general population of TAF-derived cells and/or TAF-MSC cells. In some examples, the below non-exclusive list of surface markers may be more highly expressed on the surface of neural-TAF cells as compared to other cell types, such as other TAF-derived cells and/or TAF-MSC cells:

TABLE 6
Group E markers.

| | | |
|---|---|---|
| 1. | HAVCR1 | hepatitis A virus cellular receptor 1; |
| 2. | ACKR3 | atypical chemokine receptor 3; |
| 3. | OSCAR | osteoclast associated Ig-like receptor; |
| 4. | C3 | complement C3 |
| 5. | SIRPB1 | signal regulatory protein beta 1; |
| 6. | SLC6A6 | solute carrier family 6 member 6; |
| 7. | CCKAR | cholecystokinin A receptor; |
| 8. | TNFSF10 | TNF superfamily member 10; |
| 9. | CLSTN2 | calsyntenin 2; |
| 10. | TENM2 | teneurin transmembrane protein 2; |
| 11. | SFRP1 | secreted frizzled related protein 1; |
| 12. | PIK3IP1 | phosphoinositide-3-kinase interacting protein 1; |
| 13. | SCNN1D | sodium channel epithelial 1 delta subunit; |
| 14. | CLDN11 | claudin 11; |
| 15. | ALDH3B1 | aldehyde dehydrogenase 3 family member B1; and/or |
| 16. | ITGB4 | integrin subunit beta 4 |

As will be understood by one of skill in the art, suitable combinations of the markers listed in Table 6 may be used to separate neural TAF MSCs from TAF-MSCs by selecting for specific markers from Table 6 or combinations of two, three, four, five, six or more markers from Table 6. In certain examples, neural TAF MSCs can be more specifically identified by identifying a combination of stronger expression, such as 3-fold or more stronger expression (optionally with TPM threshold >500) of any combination of the foregoing markers, e.g., HAVCR1 and/or ACKR3 and/or OSCAR and/or C3 and/or SIRPB1 and/or SLC6A6 as compared to TAF-MSCs. When using combinations of markers, identification may be achieved with a lower threshold of stronger expression, such as 2-fold or more or a higher threshold such as 6-fold or more, 8-fold or more, or 12-fold or more expression of each of the markers. In addition, those skilled in the art will also recognize that combinations including both negative and positive markers, such as at any of the thresholds described above, can also be effective to more specifically isolate neural TAF MSCs.

All of the features disclosed in this specification (including any accompanying exhibits, claims, abstract and drawings), and/or all of the steps of any method or process so disclosed, may be combined in any combination, except combinations where at least some of such features and/or steps are mutually exclusive. The disclosure is not restricted to the details of any foregoing examples. The disclosure extends to any novel one, or any novel combination, of the features disclosed in this specification (including any accompanying claims, abstract and drawings), or to any novel one, or any novel combination, of the steps of any method or process so disclosed.

Those skilled in the art will appreciate that in some examples, the actual steps taken in the processes illustrated or disclosed may differ from those shown in the figures. Depending on the example, certain of the steps described above may be removed, others may be added. For example, the actual steps or order of steps taken in the disclosed processes may differ from those shown in the figure. Depending on the example, certain of the steps described above may be removed, others may be added. Furthermore, the features and attributes of the specific examples disclosed above may be combined in different ways to form additional examples, all of which fall within the scope of the present disclosure.

Conditional language, such as “can”, “could”, “might”, or “may”, unless specifically stated otherwise, or otherwise understood within the context as used, is generally intended to convey that certain examples include, while other examples do not include, certain features, elements, or steps. Thus, such conditional language is not generally intended to imply that features, elements, or steps are in any way required for one or more examples or that one or more examples necessarily include logic for deciding, with or without user input or prompting, whether these features, elements, or steps are included or are to be performed in any particular example. The terms “comprising”, “including”, “having”, and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise, the term “and/or” in reference to a list of two or more items, covers all of the following interpretations of the word: any one of the items in the list, all of the items in the list, and any combination of the items in the list. Further, the term “each”, as used herein, in addition to having its ordinary meaning, can mean any subset of a set of elements to which the term “each” is applied. Additionally, the words “herein”, “above”, “below”, and words of similar import, when used in this application, refer to this application as a whole and not to any particular portions of this application.

Conjunctive language such as the phrase “at least one of X, Y, and Z”, unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately”, “about”, “generally”, and “substantially” as used herein represent a value, amount, or characteristic close to the stated value, amount, or characteristic that still performs a desired function or achieves a desired result. For example, the terms “approximately”, “about”, “generally”, and “substantially” may refer to an amount that is within less than 10% of, within less than 5% of, within less than 1% of, within less than 0.1% of, and within less than 0.01% of the stated amount. As another example, in certain examples, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain examples of the disclosure are encompassed in the claim set listed below or presented in the future.

As used herein, the terms “treat”, “treatment”, “treating”, or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilised (i.e., not worsening) state of allograft function (e.g. lung allograft), delay or slowing of organ function, and amelioration or palliation of symptoms associated with end stage organ disease.

A subject can be one who has been previously diagnosed with or identified as suffering from or having a condition in need of treatment (e.g., lung failure) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition, or one or more complications related to the condition or a subject who does not exhibit risk factors. For example, a subject can be one who exhibits one or more symptoms for a condition, or one or more complications related to the condition or a subject who does not exhibit symptoms. A “subject in need” of diagnosis or treatment for a particular condition can be a subject suspected of having that condition, diagnosed as having that condition, already treated or being treated for that condition, not treated for that condition, or at risk of developing that condition.

A therapeutically or prophylactically significant reduction in a symptom is, e.g., at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject or the state of the subject prior to administering isolated TAF MSCs. Measured or measurable parameters include clinically detectable markers of disease, for example, elevated or depressed levels of a biological marker, as well as parameters related to a clinically accepted scale of symptoms or markers for fibrosis and/or inflammation. It will be understood, however, that the total daily usage of the compositions and formulations as disclosed herein will be decided by the attending physician within the scope of sound medical judgment. The exact amount required will vary depending on factors such as the type of disease being treated, gender, age, and weight of the subject.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. Embodiments and features of the present invention are also outlined in the following items and also illustrated by the following non-limiting examples.

Items

Item 1. Isolated term amniotic fluid (TAF) mesenchymal stem cells (MSCs) for use in treating low blood oxygenation levels in a subject via extracorporeal membrane oxygenation (ECMO).

Item 2. The isolated TAF MSCs according to Item 1, wherein the isolated TAF MSCs are administered before, after, and/or during ECMO.

Item 3. A composition comprising isolated term amniotic fluid (TAF) mesenchymal stem cells (MSCs) for use in treating low blood oxygenation levels in a subject via extracorporeal membrane oxygenation (ECMO).

Item 4. The composition according to Item 3, wherein the composition is administered before, after, and/or during ECMO.

Item 5. Isolated TAF MSCs for use in treating acute respiratory distress syndrome (ARDS) in a subject via ECMO.

Item 6. A composition comprising isolated TAF MSCs for use in treating ARDS in a subject via ECMO.

Item 7. An extracorporeal blood treatment system for treating a patient, the system comprising:
an extracorporeal blood circuit;
a processing fluid circuit;
said extracorporeal blood circuit and processing fluid circuit being divided by an oxygenation membrane of a filtration unit;
at least one blood pump for controlling the flow of blood through the blood circuit;
at least one processing fluid pump for controlling the flow of processing fluid through the processing fluid circuit;
a system computing unit operatively connected to the blood pump and the processing fluid pump, said system computing unit having at least one input means (e.g. a keyboard, touch screen or sensor); wherein the system computing unit is adapted for receiving a desired blood oxygenation value O_{b};
the system computing unit is adapted for receiving an actual blood oxygenation value Oₐ;
the system computing unit being adapted for controlling said blood pump and said processing fluid pump so as the actual blood oxygenation value Oₐ is driven towards the desired blood oxygenation value O_{b};
the system is adapted for receiving isolated TAF MSCs.

Item 8. The system according to Item 7, wherein the system is adapted for receiving:
(i) 20 million isolated TAF MSCs per minute;
(ii) isolated TAF MSCs before and/or after the oxygenation membrane;
(iii) a desired highest blood concentration of isolated TAF MSCs;
(iv) a desired highest oxygenation membrane pressure;
(v) an initial infusion rate of 7.5-20.0 units/kg/h of heparin.

Item 9. A formulation for use in the system according to any one of Items 7 or 8, wherein the formulation comprises isolated TAF MSCs and (dimethyl sulfoxide) DMSO.

Item 10. A method of oxygenating a blood sample in the presence of isolated TAF MSCs.

Item 11. The use, method or system according to any one of the preceding Items, wherein the number of isolated TAF MSCs is at least 1 million cells per kg of the subject/patient, for example at least 2, 3, 4, 5, 6, 7, 8, 9, 10, 11, 12, 13, 14, 15, 16, 17, 18, 19 or 20 million cells per kg of the subject/patient.

Item 12. The use, method or system according to any one of the preceding Items, wherein the isolated TAF MSCs are introduced before, during and/or after the blood sample contacts the membrane.

Item 13. The use, method or system according to any one of the preceding Items, further comprising an anticoagulant, preferably a low molecular weight anticoagulant.

Item 14. The use, method or system according to any one of the preceding Items, wherein the anti-coagulant is heparin, for example a low molecular weight heparin.

Item 15. The use, method or system according to any one of the preceding Items wherein the isolated TAF MSCs are:
a. a clonal population;
b. a mix of clonal populations;
c. heterogeneous/homogeneous;
d. in a single-cell suspension/pelleted;
e. are capable of forming colony forming units (CFU) in culture;
f. functionally characterised (e.g. release of cytokines implicated in growth stimulation or suppression; differentiation into osteogenic, chondrogenic or adipogenic cell lineages);
g. have been pre-sorted or enriched to contain markers of interest;
h. passaged 1, 2, 3, 4, 5, 6, etc times; and/or
i. in a frozen state (and require thawing before use).

Item 16 The use, method or system according to any one of the preceding Items, wherein the isolated TAF MSCs comprise (or have been enriched/selected to comprise):
(i) at least one surface marker selected from the group consisting of TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2;
(ii) at least one surface marker selected from the group consisting of PCDH19, DDR1, MME, IFITM10, BGN, NOTCH3, SULF1, TNFSF18, BDKRB1, FLT1, PDGFRA, TNFSF4, UNC5B, FAP, CASP1, CD248, DDR2, PCDH18, LRRC38, and CRLF1;
(iii) at least one surface marker selected from the group consisting of HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1;
(iv) at least one surface marker selected from the group consisting of TNFSF18, PCDH19, NCAM2, TNFSF4, CD248, DDR2, HTR2B, PCDH18, SULF1, MME, ADGRA2, DCSTAMP, PDGFRA, UNC5B, SCUBE3, CEMIP, BDKRB1, FLT1, BDKRB2, FAP, CASP1, and SRPX2; or
(v) at least one surface marker selected from the group consisting of HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1, and ITGB4.

Item 17. The use, method or system according to any one of the preceding Items, wherein the isolated TAF MSCs average size is between 15-25 μm diameter, preferably between 18-22 μm diameter.

Item 18. The use, method or system according to any one of the preceding Items, wherein the isolated TAF MSCs comprise lower actin expression or fewer vesicles at the surface compared with adult MSCs.

Item 19. The use, method or system according to any preceding Items, wherein are at least 10%, 20%, 30%, 40%, 50%, 60%, 70%, 80%, 90%, 95%, 96%, 97%, 98% or 99% of the TAF MSCs are lung TAF MSCs.

### EXAMPLES

### Example 1—Measurement of the Formation of TAF MSCS Aggregates in an ECMO Setting

### Materials and Methods

Unsorted TAF MSCs were spiked into artificial blood running through an ECMO filter and TAF MSC size was measured. Tubes with 3 ml of blood in each were provided and subjected to the following procedure:
1. Transferred 3 ml blood to a 50 ml falcon tube.
2. Added 30 ml of ACK lysis buffer*.
3. Incubated at room temperature (RT) for 5 minutes.
4. Centrifuged at 300×g for 5 minutes at RT.
5. Decanted the supernatant carefully without disturbing the pellet.
6. Added 5 ml of PBS and resuspended properly.
7. Centrifuged at 300×g for 5 minutes at RT.
8. Decanted the supernatant carefully without disturbing the pellet.
9. Resuspended the pellet in 200 μl of PBS.
10. Counted cell number using Nucleocounter 202 (NC-202; Chemometec). *1 ml of blood demands 10-20 ml of lysis buffer (10 ml lysis buffer/1 ml blood was used).

For the NC-202, at least 50,000 cells/ml of cell suspension were analysed. The NC-202 input is 200 μl, equating to at least 10,000 cells present in the 200 μl cell suspension to get a reliable value on the NC-202. Therefore, there should be 3,500 cells/ml of blood if the blood volume provided for analysis is 3 ml.

### Results

Table 1 provides the measurements of the TAF MSCs spiked in blood running through an ECMO filter.

TABLE 1
Measurement of TAF MSCs by NC-202

| | Total no: of cells/ | No: of live | | | % |
|---|---|---|---|---|---|
| Sample | ml of blood | cells/ml | % | Diameter | Aggre- |
| ID | (live + dead) | blood | viability | (μm) | gates |
| T0 | 667 | 520 | 77.96 | 20.1 | 0 |
| T30 | 1505 | 1248 | 82.92 | 18.7 | 0 |
| T60 | 520 | 286 | 55.00 | 20.3 | 0 |
| T90 | 750 | 286 | 38.13 | 21.4 | 0 |
| T120 | 917 | 750 | 81.78 | 18.3 | 0 |
| T150 | 417 | 417 | 100.00 | 21.4 | 0 |
| T240 | 1367 | 538 | 39.35 | 18.3 | 0 |

The Sample ID corresponds to a particular time point, wherein T=time and the subsequent numerical value is the number of minutes. For example, T30 indicates a 30-minute duration of ECMO, and T120 indicates a 2-hour duration of ECMO. It is expected that both the total number of cells and the total number of live cells/ml blood fluctuates. Due to the small number of TAF MSC's per ml blood the dilution of TAF MSC's is high. Due to the high dilution and due to the nature of ECMO system the TAF MSC's are not equally distributed in the system and this is reflected as fluctuation of the total number of cells and the total number of live cells/ml in Table 1.

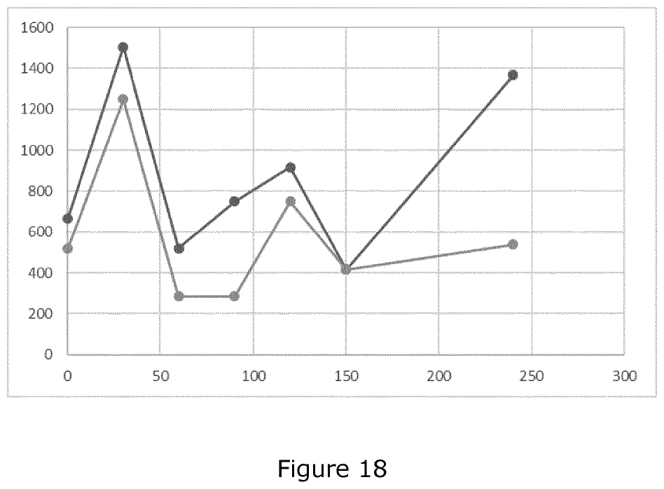

The results in Table 1 are plotted graphically in FIG. 18. The diameter of the TAF MSCs is observed to range from 18.3-21.4 μm. This range of diameters is expected for TAF MSCs, and so is a good indication of the success of the cell count and analysis following ECMO. Although the number of cells per ml of blood fluctuates, it is clear that cells are present throughout the measurement period and are not declining over time. As disclosed in Table 1 no aggregates can be measured at any of the time points T0-T240. Moreover, no change in pressure over the membrane was observed. Accordingly, even at 4 hours (i.e. T240), the flow of cells in the system is not obstructed. The data therefore show that TAF MSCs used in an ECMO system do not clog the oxygenator membrane even up to 4 hours of flow in the ECMO system.

### Example 2—Effect on TAF MSC's on T-Cell Activation and Macrophage Activation/Polarization Using Human Peripheral Blood Mononuclear Cells (PBMCs)

### 1.1 Aim

The aim of the study was to evaluate the effect of unsorted human Mesenchymal StemCells (MSCs) on T cell activation and macrophage activation/polarization using human Peripheral Blood Mononuclear Cells (PBMCs).

### 1.2 Ex Vivo Analysis

Freshly isolated human PBMCs, pooled from three donors, were activated with anti-CD3/anti-CD28 at 10 μg/ml and 5 μg/ml respectively in presence of MSCs ex vivo for 24 or 72 hours. Following activation, cells were analysed for effects on cellular composition after 24 hours and for effects on cellular composition and cell proliferation using CFSE after 72 hours. In addition, supernatants collected after 24 hours of activation were analysed for cytokine levels using Luminex.

### 2. Materials and Methods

### 2.1 Reagents

AbC Total Compensation capture beads (Life Technologies, A10497)
AD-MSCs (provided by Sponsor)
Anti-CD3 (Nordic Biosite, 300438)
Anti-CD28 (Nordic Biosite, 302934)
Anti-human CD4—PerCp (Nordic Biosite, 344624)
Anti-human CD8—QDot800 (Thermo Fisher, Q22157)
Anti-human CD80—BV421 (Nordic Biosite, 305222)
Anti-human CD73—FITC (Nordic Biosite, AM26144FC-N)
Anti-human PD-1—PE (Nordic Biosite, 329906)
Anti-human CD163—BV605 (Nordic Biosite, 333616)
Anti-human CD206—BV711 (Nordic Biosite, 321136)
Cell culture medium (StemMACS MSC expansion Media, Miltenyi (M3))
Cell culture medium (Prime-XV MSC Expansion XSFM, IrvineScientific (M4))
Carboxyfluorescein succinimidyl ester (CFSE), (Sigma, 21888)
HBSS (Gibco, Life Technologies, 14175)
Human MSCs (provided by Sponsor)
Human PBMCs (Blodcdentralen, Lunds Universitetssjukhus, Lund)
Luminex 12-plex (RnD Systems, LXSAHM-12)
mqH20 (QPAK1, Millipore)
Negative beads (Life Technologies, A10497)
PBS (Gibco, Life Technologies, 14190)

### 2.2 Equipment

Attune Nxt (ThermoFisher Scientific, Sunnyvale, California, USA)
Scepter cell counter (Millipore Merck, MA, USA) SpectraMax
Luminex 200 (Bio-Rad, Solna, Seden)
Thermo scientific cell culture plate 96-well U bottom (Thermo Fisher, 168136)
V-bottom plates (Nunc, 732-0191)
Falcon Tubes (VWR, 734-0443)

### 2.3 Procedures

### 2.3.1 Ex Vivo Assay

Peripheral Blood Mononuclear Cells (PBMCs) were isolated from leucocyte concentrate from three different donors (acquired from Blodcentralen, Lunds Universitetssjukhus, Lund, Sweden) through gradient centrifugation at 400×g for 40 minutes using Ficoll. Red blood cells (RBCs) were lysed using BD Pharmlyse buffer 10× diluted in milliQ water for 3 minutes at room temperature (RT). Cells were washed and the isolated PBMCs were pooled. Cell concentration was determined using a Scepter cell counter (Millipore Merck, MA. USA). Cells were diluted to 1×10⁷ cells/ml in PBS. Pooled PBMCs were split into 2 different tubes. Cells in tube 1 was stained with CFSE at 5 μM for 5 minutes (dark, RT). CFSE stained cells were washed with an equal volume of FBS to stop the reaction and washed again with PBS. Cells in tube two was left in PBS.

MSCs (donor 1, donor 2, donor 3, and AD-MSC control cells) were provided in 8 different tubes, two for each cell type (in different medias (M3 and M4). All cells (PBMCs and MSCs) were diluted in the two different cell culture media (M3 and M4) to a final concentration of 2*10⁶ cells/ml. Media was supplemented with anti-CD28 at 5 μg/ml. Cells were added to anti-CD3 (1 μg/well, 100 μl/well) coated U bottom cell culture plates according to the layout below, 200 μl/well in different PBMC:MSC ratios (1:10, 1:5, 1:2.5, 1:0 and 0:1) with a total amount of cells at 4*10⁵ cells/well.

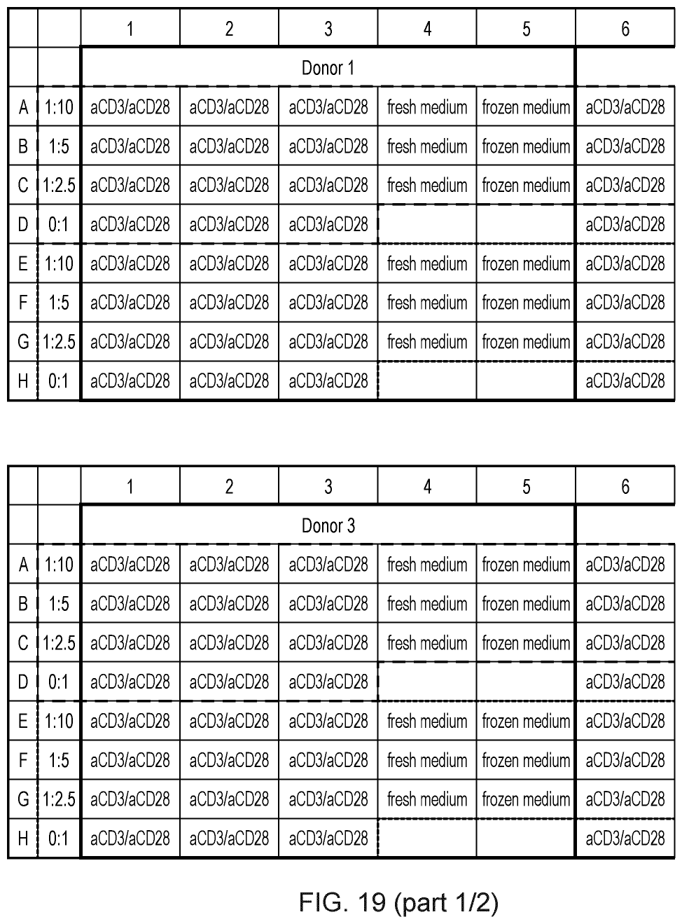

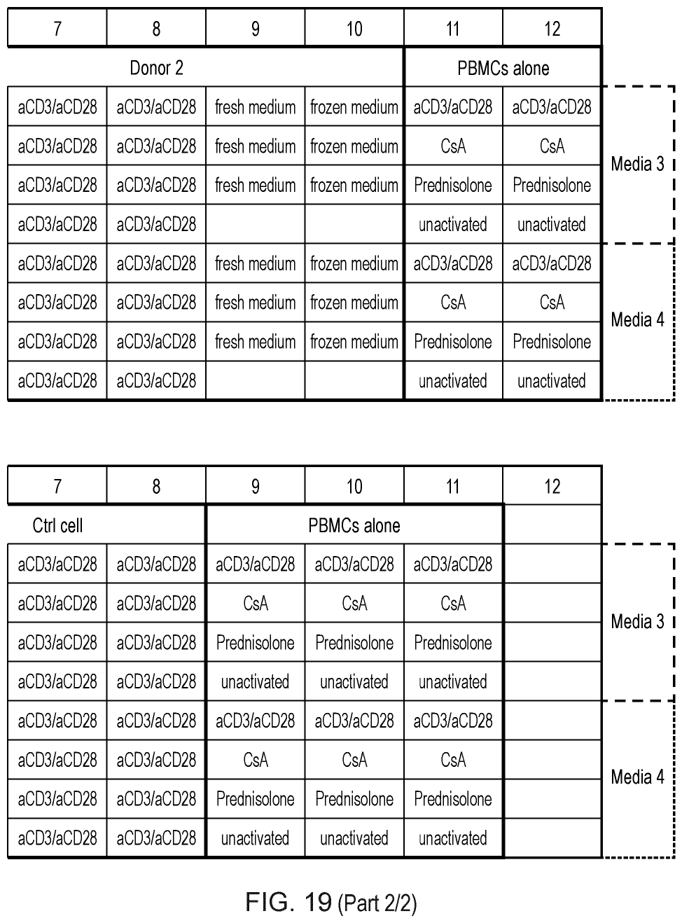

Included in the assay were also controls (stimulated and unstimulated PBMCs) and two reference drugs, e.g. cyclosporine (CsA) and Prednisolone (Pred). When plating controls, cells were spun down and diluted in M3 or M4 at 4*10⁶ cells/ml, with a total amount of cells at 4*10⁵ cells/well. 100 μl PBMCs were added per well. CsA was added at a final concentration of 10 μg/ml and Prednisolone was added at a final concentration of 125 nM (700 μl/well). Medium was added to unstimulated control wells (700 μl/well). Cells were incubated for 24 or 72 hours at 37° C., 5% CO₂. See also FIG. 19.

2.3.2 Cell Composition after 24 Hours of Activation (FACS Analysis)

Following 24 hours of incubation, cells in FACS/Luminex plates were stained with antibodies detecting CD73, CD4, CD8, CD80, CD206, CD163 and PD-1 in staining buffer (PBS supplemented with 1% BSA). Briefly, cells were transferred to V bottom plates and centrifuged at 360×g for 2 min. Supernatant was transferred to a storage plate and put in −20° C. until analysis using Luminex. Cells were washed in PBS, centrifuged at 360×g for 2 min at 4° C. Supernatant was flicked off and antibodies against surface markers were added. Cells were incubated at +4° C. for 20 minutes (dark). After incubation, cells were washed 1× in PBS, centrifuged at 360×g for 2 min at 4° C. Cells were resuspended in PBS and acquired using the Attune Nxt flow cytometer. Compensation was performed using beads—AbC Total Compensation capture beads and negative beads. Since compensation was done using beads, FSC and SSC were changed before analysing samples and adjusted to cells. Lasers were not changed after compensation. 150 μl was analysed from each sample.

2.3.3 Proliferation and Cell Composition after 72 Hours of Activation (FACS Analysis)

After 72 hours of incubation, CFSE stained cells were stained with CD4, CD8, CD80, CD206 and CD163 in staining buffer. Briefly, cells were transferred to V bottom plates, centrifuged at 360×g for 2 min. Cells were washed in PBS, centrifuged at 360×g for 2 min at 4° C. Supernatant was flicked off and surface markers were added. Cells were incubated at +4° C. for 20 minutes. After incubation, cells were washed 1× in PBS, centrifuged at 360×g for 2 min at 4° C. Cells were resuspended in PBS and acquired using the Attune Nxt flow cytometer. Compensation was performed using beads—AbC Total Compensation capture beads and negative beads for surface markers. For CFSE compensation, newly CFSE stained cells were used. Since compensation was done using both beads and cells, FSC and SSC were changed and adjusted to beads or cells. Lasers were not changed after compensation.

2.3.4 Cytokine Analysis after 24 Hours of Activation (Luminex)

Supernatant (24 hours incubation) was analysed for cytokines using the 12-plex LXSAHM-12 Luminex kit. All reagents, standard and samples were prepared at room temperature and according to manufacturer's instruction. Briefly, 50 μl of standard or sample was added to wells. Microparticles were added to samples and standard and samples were incubated for 2 hours at room temperature on a horizontal orbital shaker (800 rpm). Plate was washed 3× in washing buffer using a magnetic plate. Biotin-antibody was added and incubated dark for 1 hour at room temperature on a horizontal orbital shaker (800 rpm). Plate was washed 3× in washing buffer using a magnetic plate. Streptavidin-PE was added to each well and incubated dark at room temperature on a horizontal orbital shaker (800 rpm) for 30 minutes. Plate was washed 3× in washing buffer using a magnetic plate. Microparticles were resuspended in 100 μl washing buffer, incubated for 2 minutes on a horizontal orbital shaker (800 rpm). Samples were analysed on a Bio-Rad Luminex analyzer.

### 2.4 Acquisition and Analysis

Graphs were performed using Prism 8 for Mac OS X (GraphPad Software, San Diego, CA, USA). Results are presented as mean values+SEM, if not otherwise stated. Dot plots were acquired from analysis using FlowJo v10.6.1 for Mac (BD, New Jersey, USA). The inhibitory effect of MSCs on PBMC proliferation was calculated according to the two formulas below:

Coculture norm prolif (%)=Coculture prolif/Stimulated single PBMC culture prolif

Inhibitory effect=100−coculture normalized prolif

Ref: Optimisation of a potency assay for the assessment of immunomodulative potential of clinical grade multipotent mesenchymal stromal cells, Irene Oliver-Vila, Received: 4 Oct. 2017/Accepted: 29 Dec. 2017/Published online: 10 Jan. 2018 6 Springer Science+Business Media B.V., part of Springer Nature 2018, Cytotechnology (2018) 70:31-44 https://doi.org/10.1007/s10616-017-0186-0

### 3. Results

3.1 Cell Composition after 24 Hours of Activations (FACS Analysis)

### 3.1.1 Gating Strategy

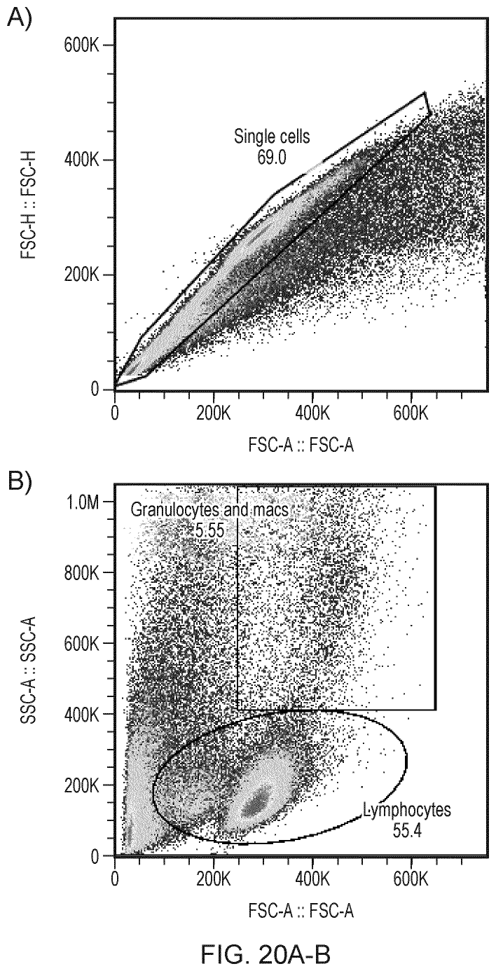

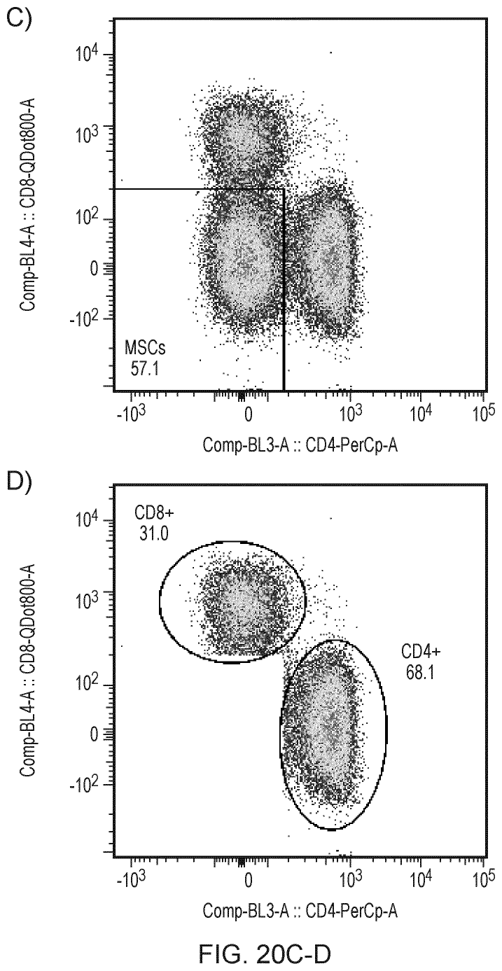

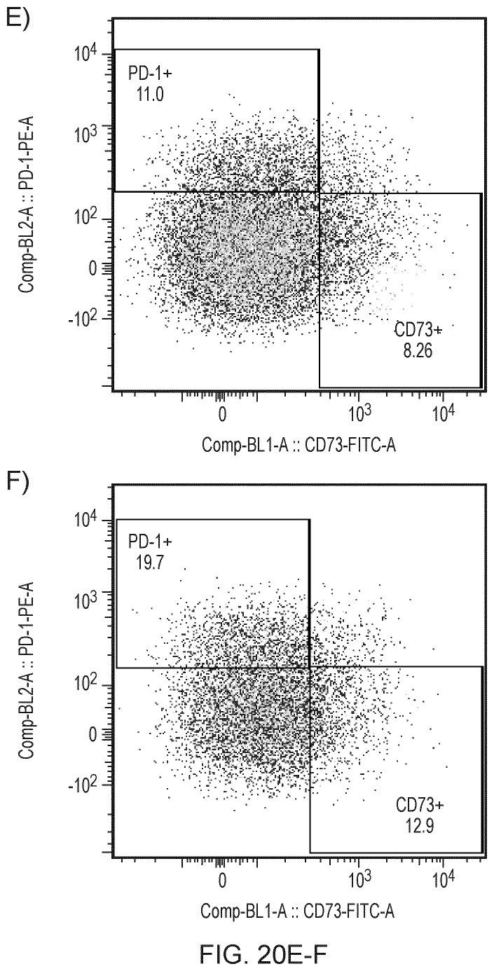

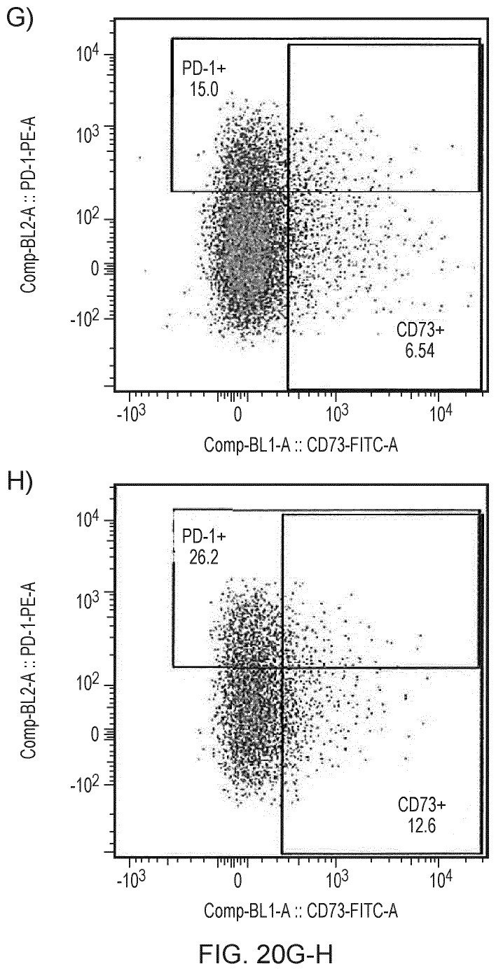

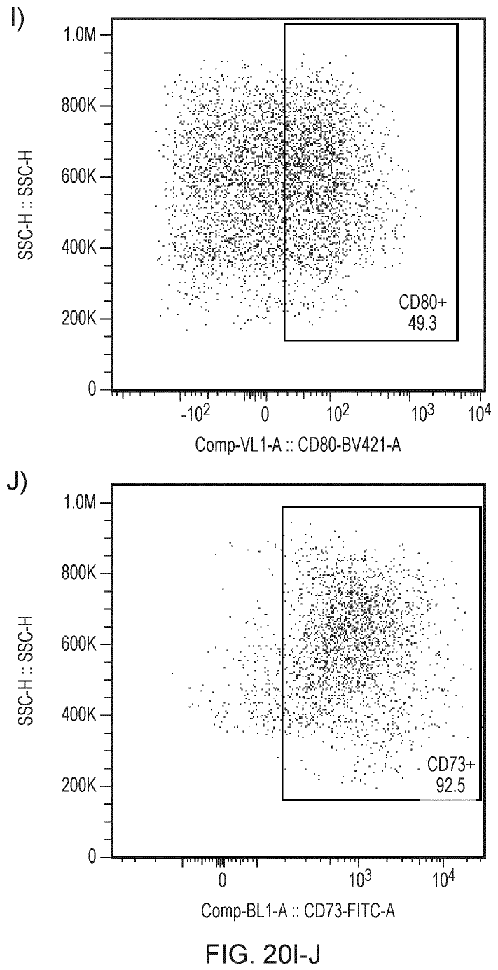

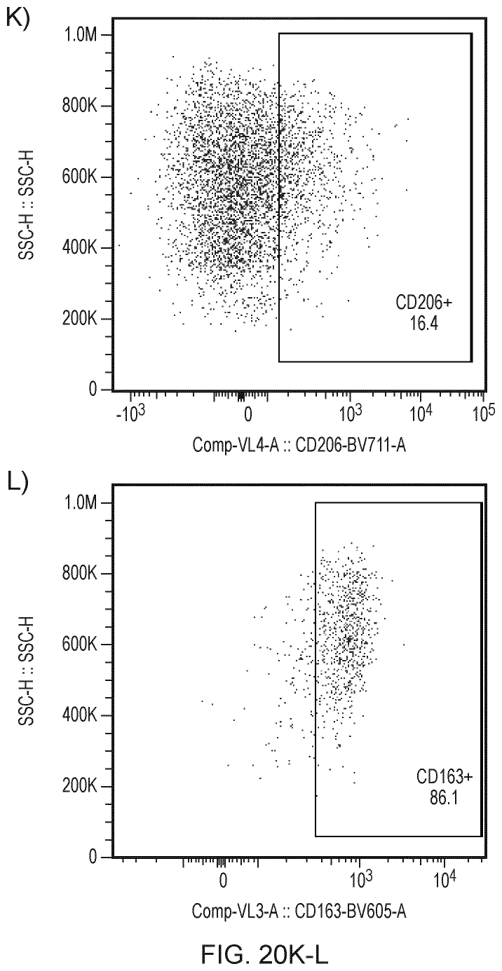

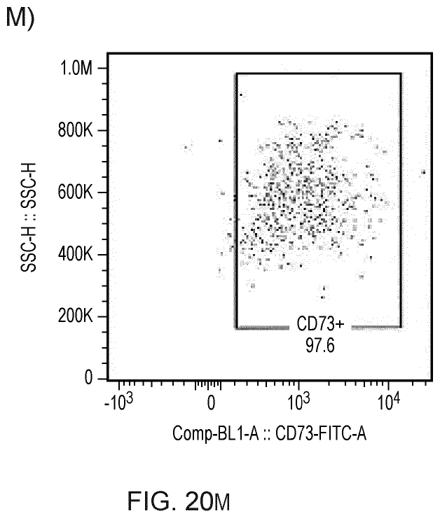

See FIG. 20

### 3.1.2 Representative Plots of Gating on Different Ratios

See FIG. 21

3.1.3 Results Cell Composition after 24 Hours of Activation—Shown for M3

Th (T helper cells) are PD-1+ cells among CD4+ cells.

Treg (Regulatory T cells) are CD73+ cells among CD4+ cells.

Tc (Cytotoxic T cells) are PD-1+ cells among CD8+ cells.

Teff (Effector T cells) are CD73+ cells among CD8+ cells.

M1 (M1 macrophages)—Macrophages that encourage inflammation.

M2 (M2 macrophages)—Macrophages that decrease inflammation and encourage tissue repair.

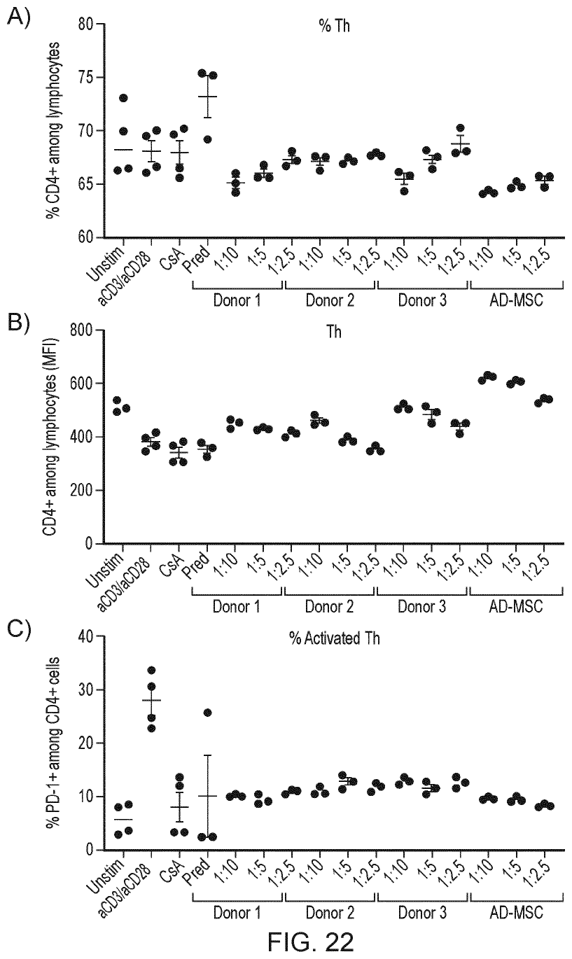

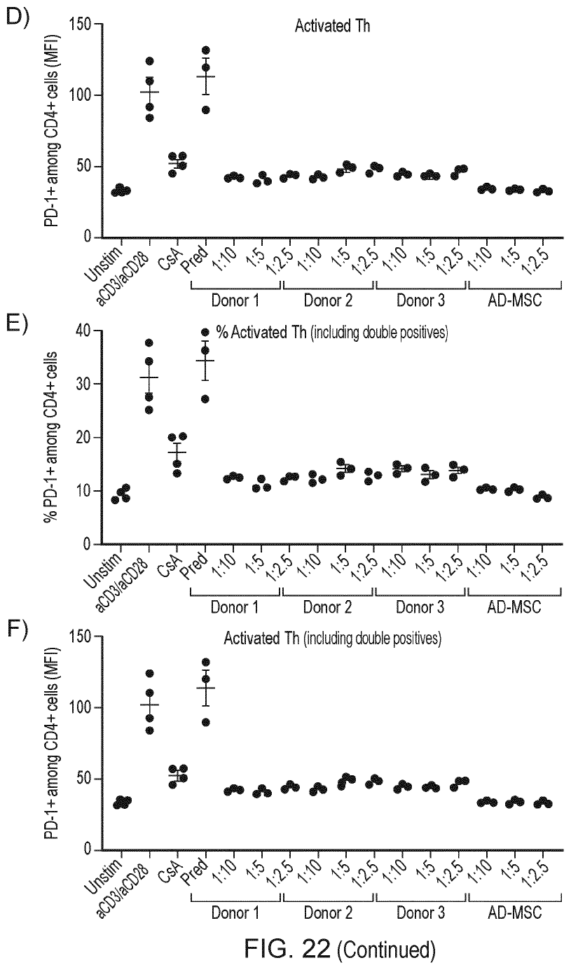

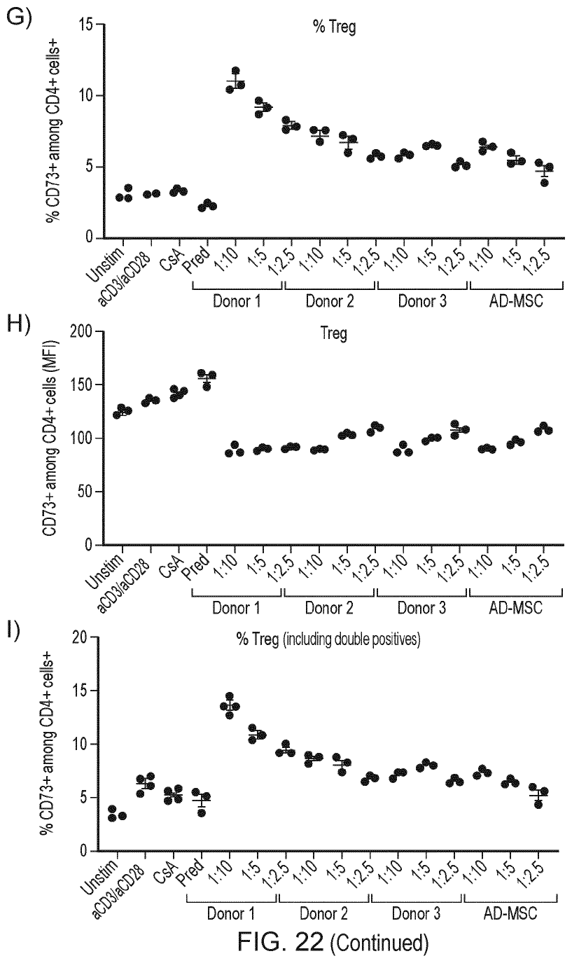

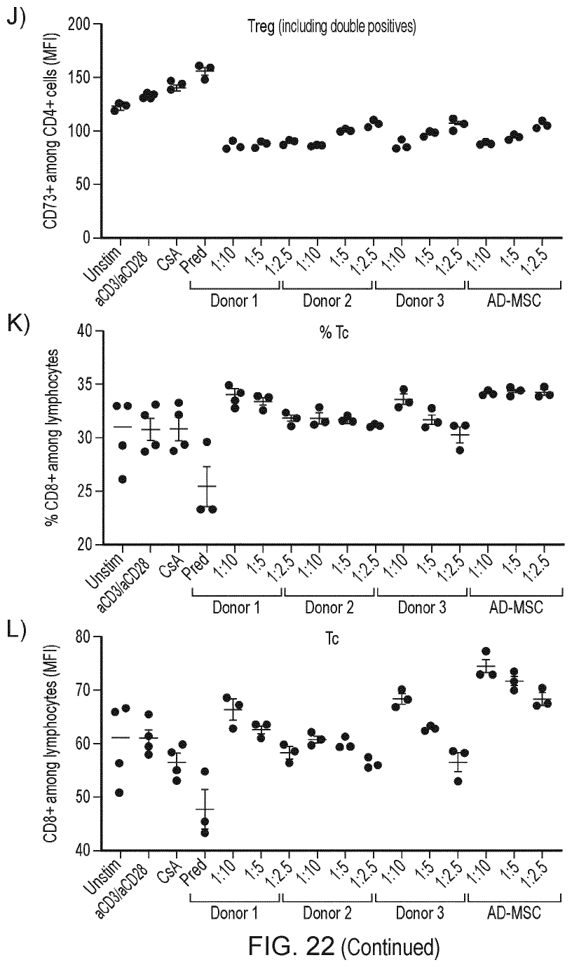

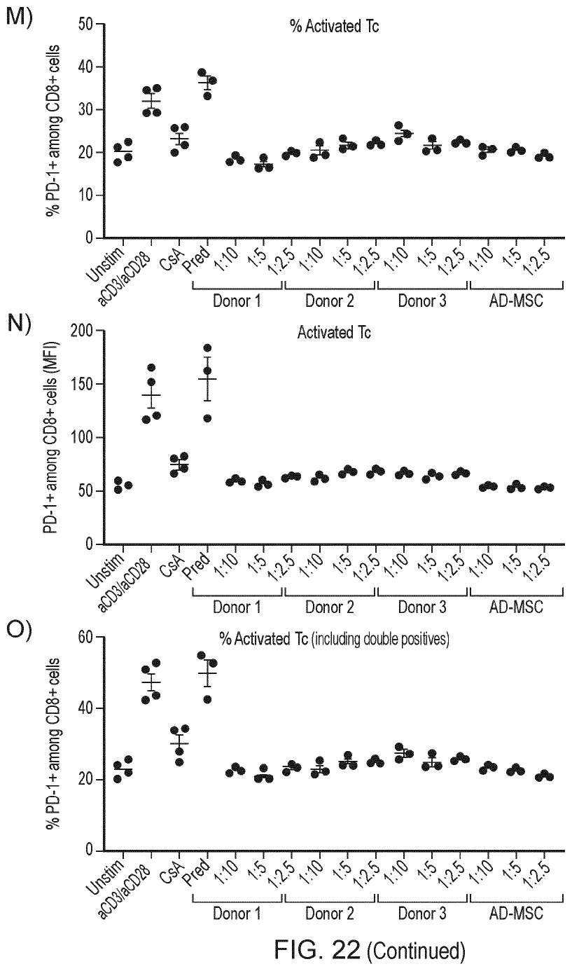

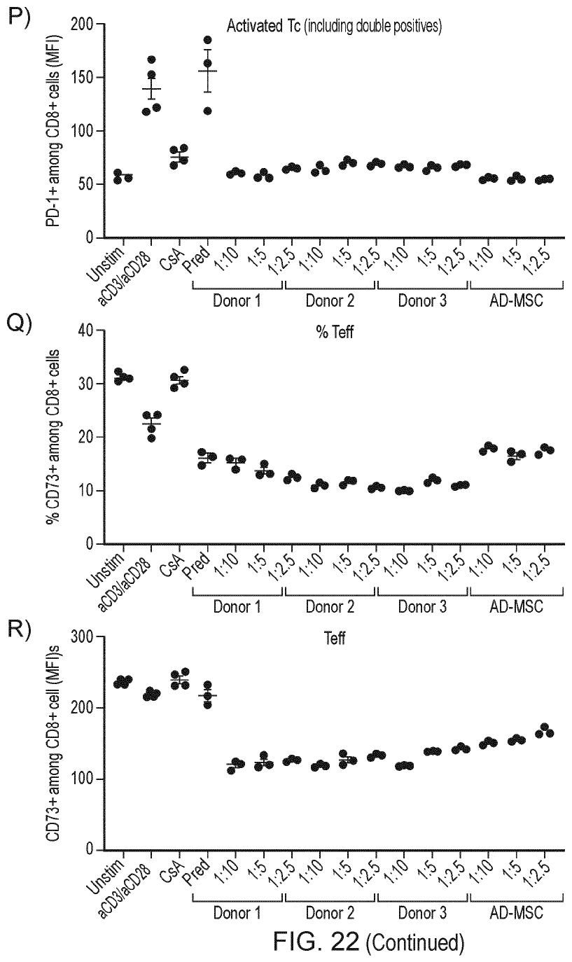

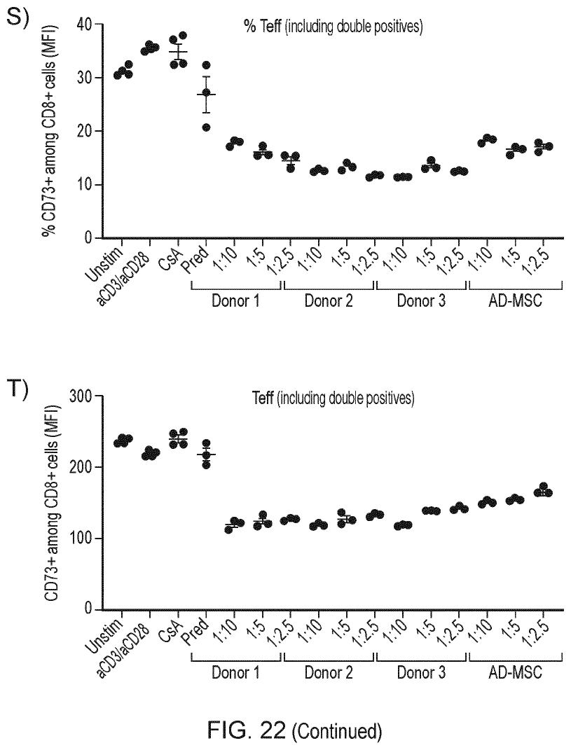

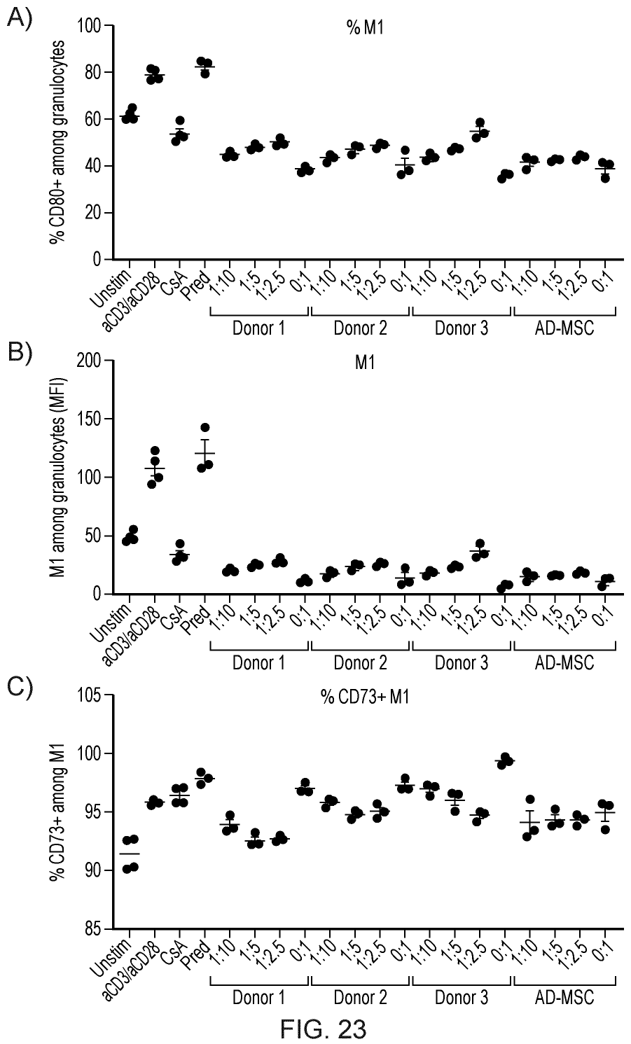

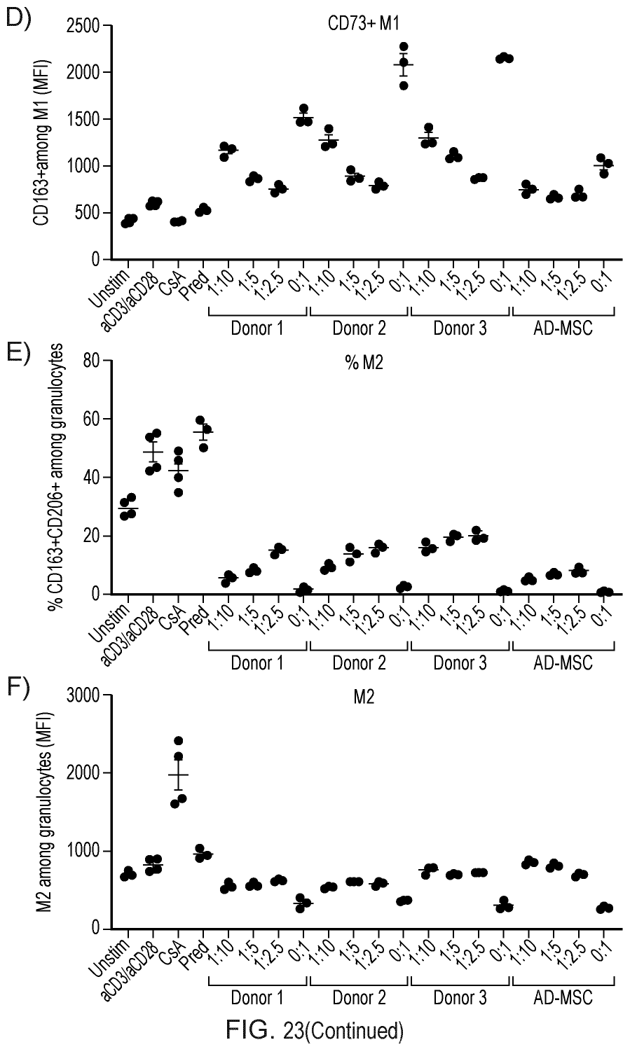

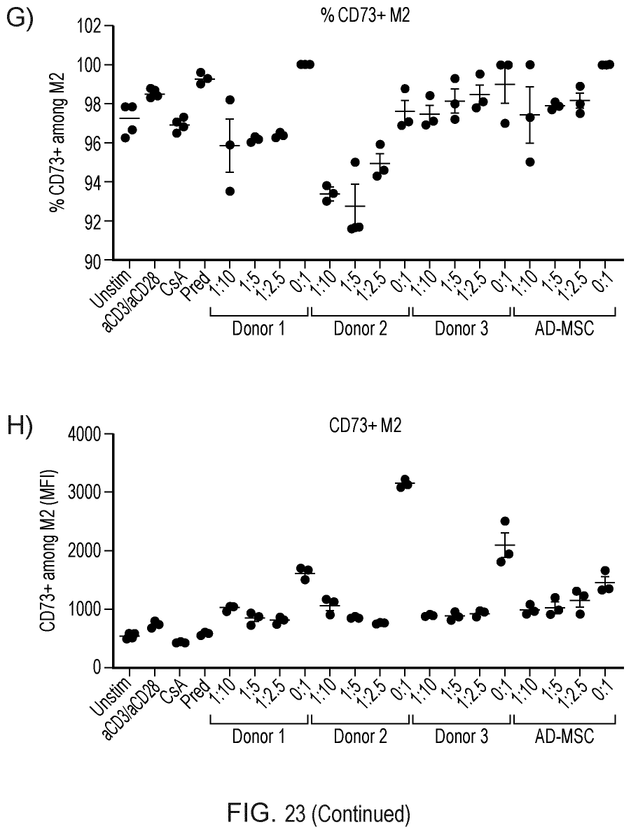

See FIG. 22 and FIG. 23

3.2 Proliferation and Cell Composition after 72 Hours of Activation (FACS Analysis after CFSE Labeling)

CFSE labeling of PBMCs was performed before coculture with MSCs, therefore all CFSE positive cells should be PBMCs.

### 3.2.1 Gating Strategy

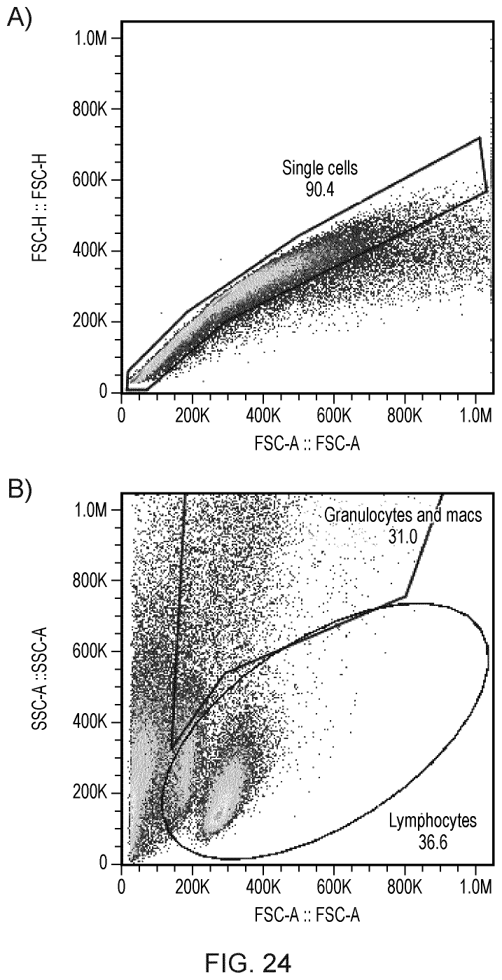

See FIG. 24

### 3.2.2 Gating Strategy for Cell Proliferation (CFSE)

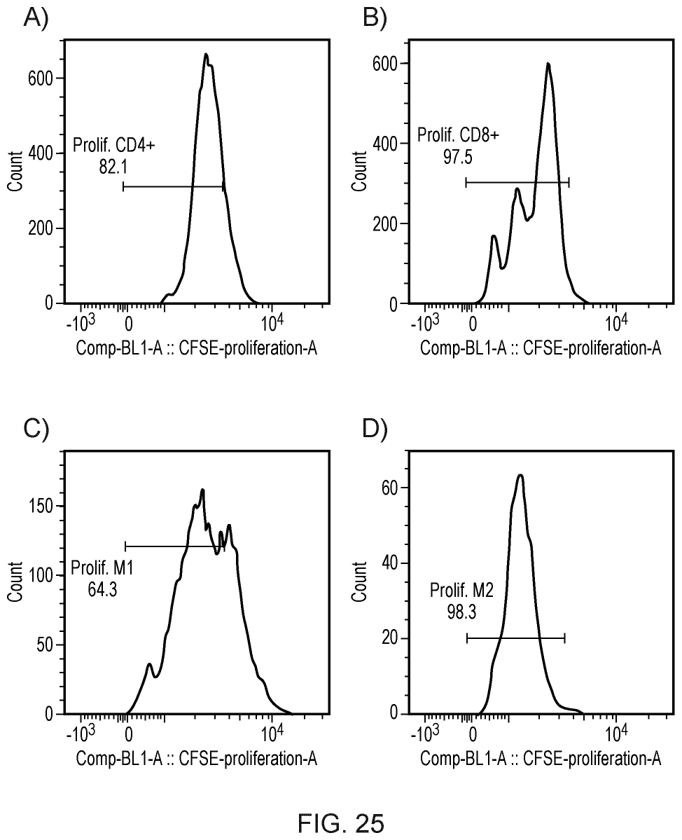

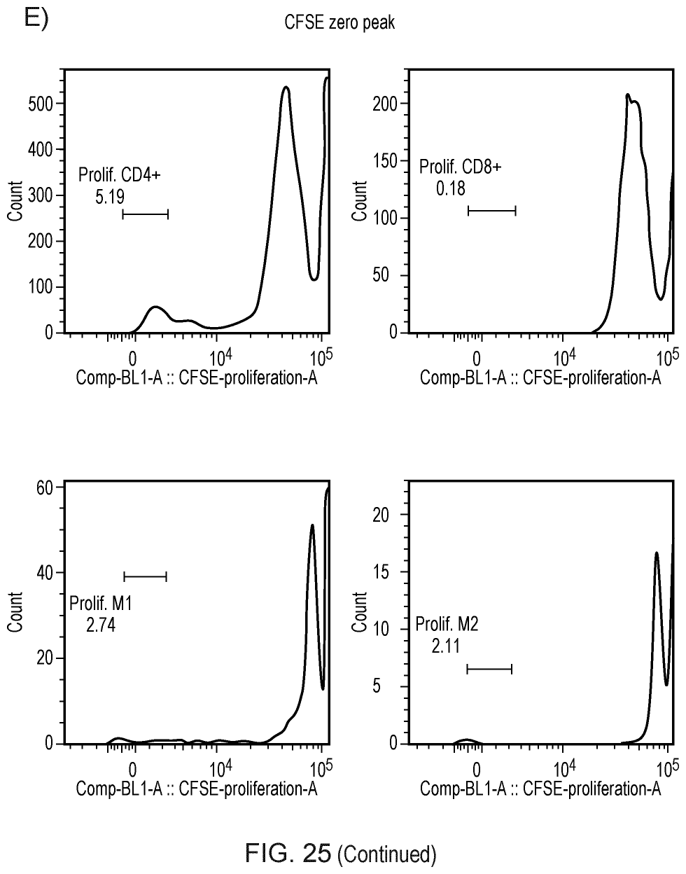

See FIG. 25A-E

3.2.3 Staining with CFSE

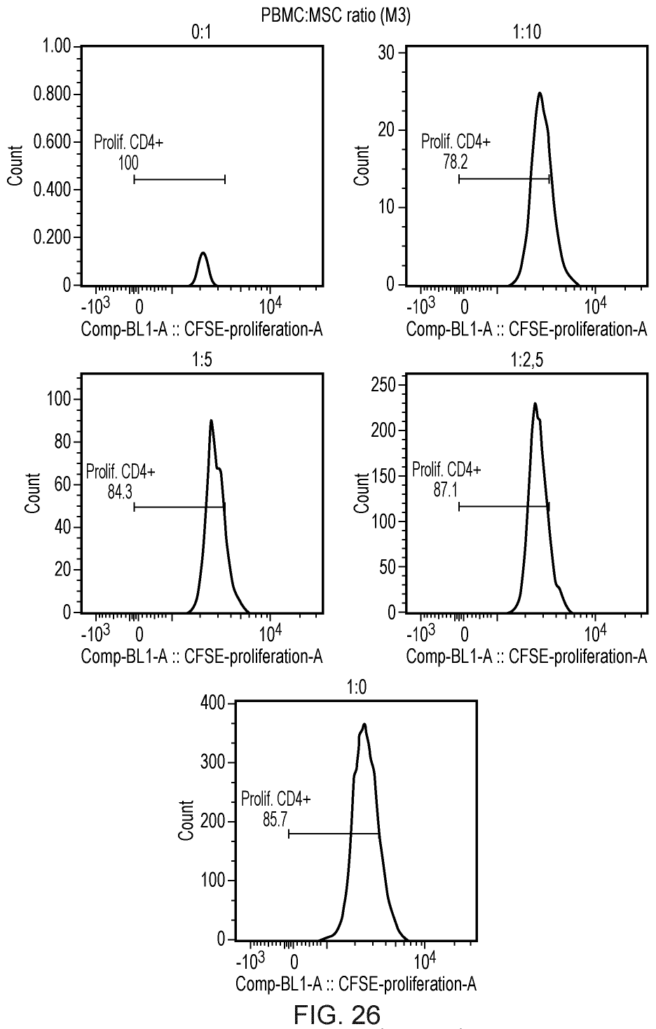

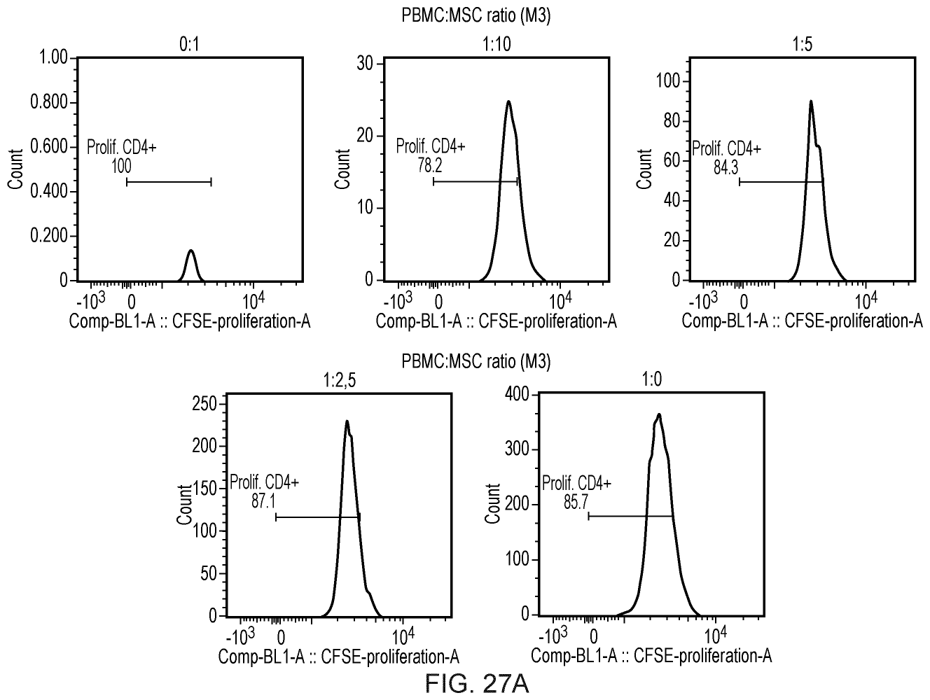

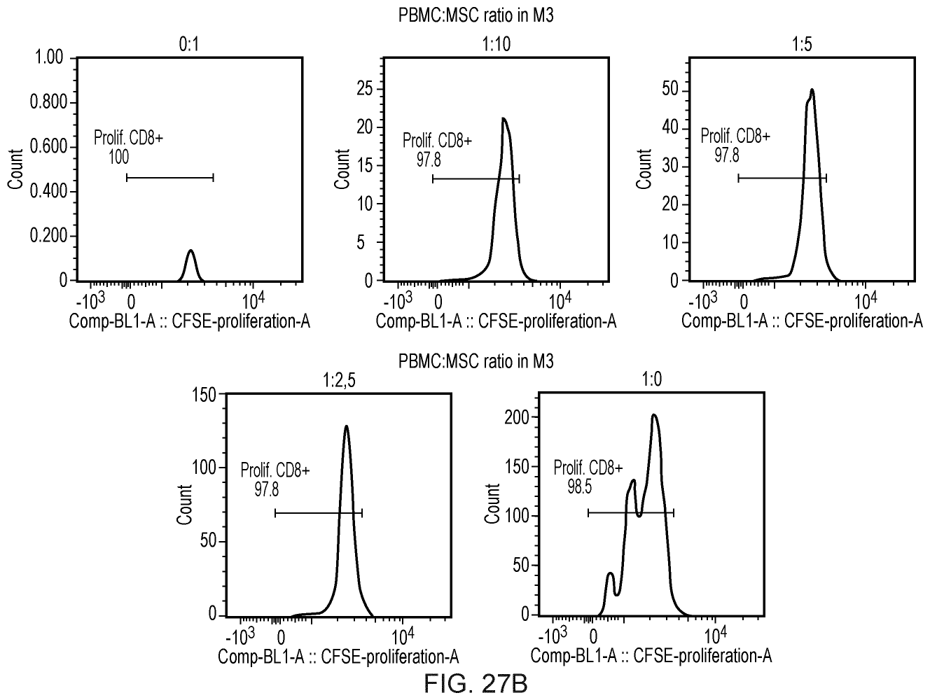

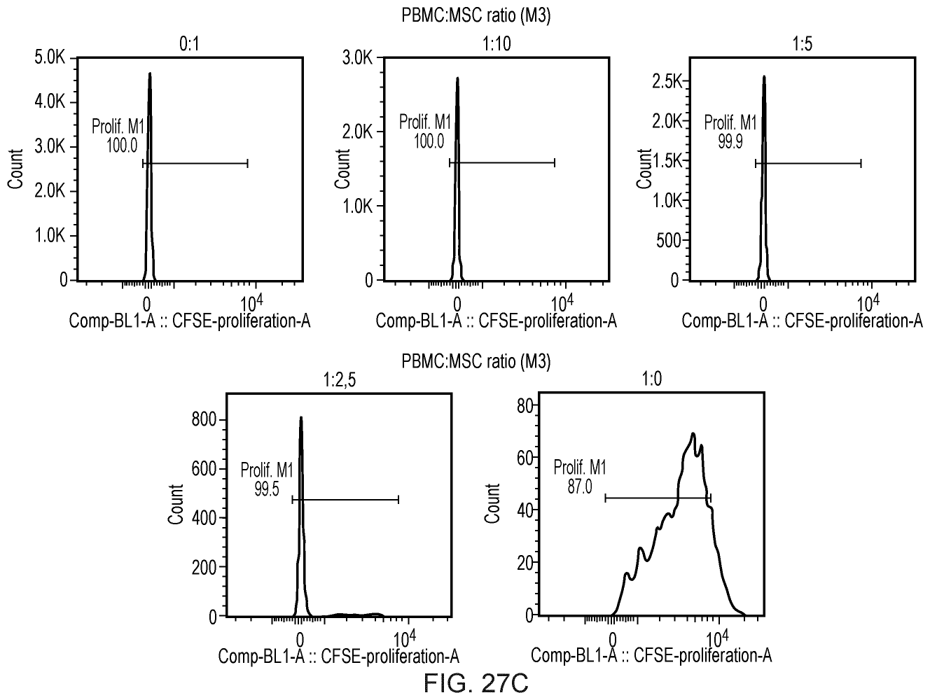

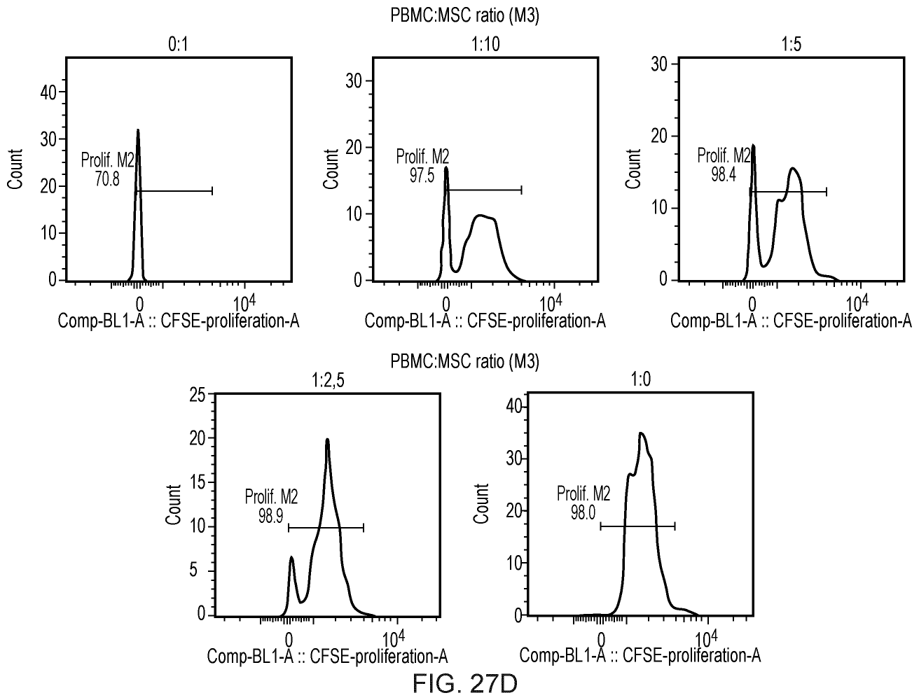

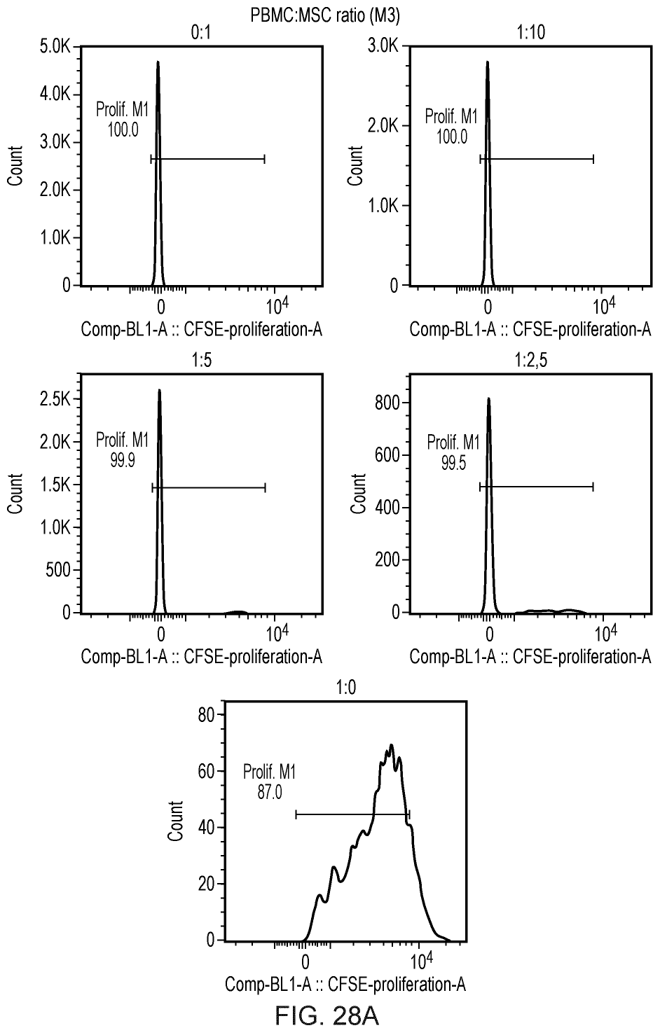

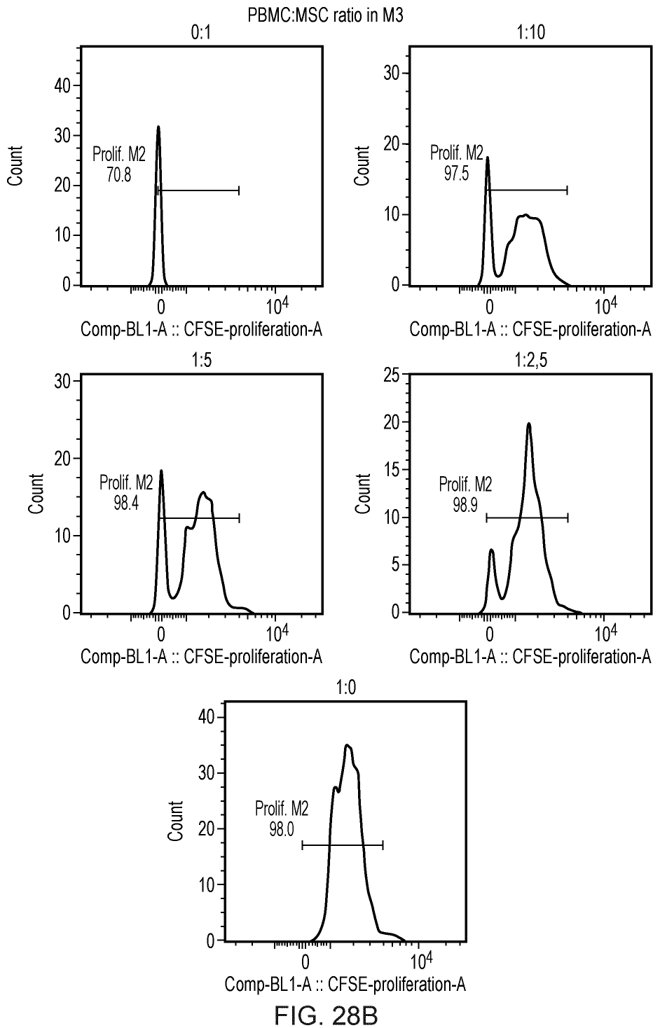

See FIG. 26-28A-B

3.2.4 Results—Proliferation and Cell Composition after 72 Hours of Activation (FACS Analysis after CFSE Labeling)—Shown for Media 3

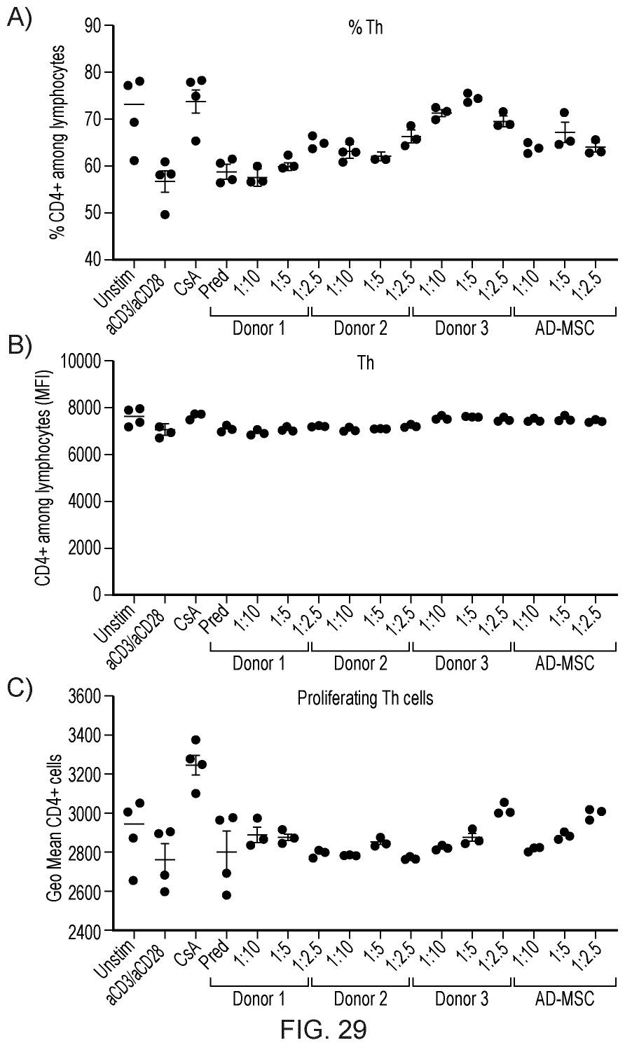

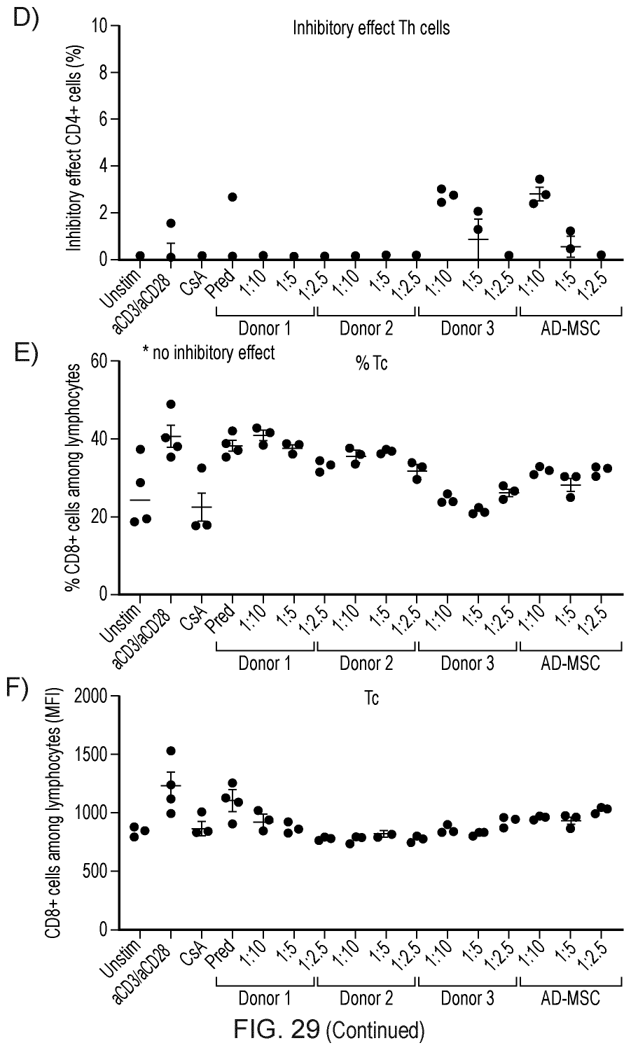

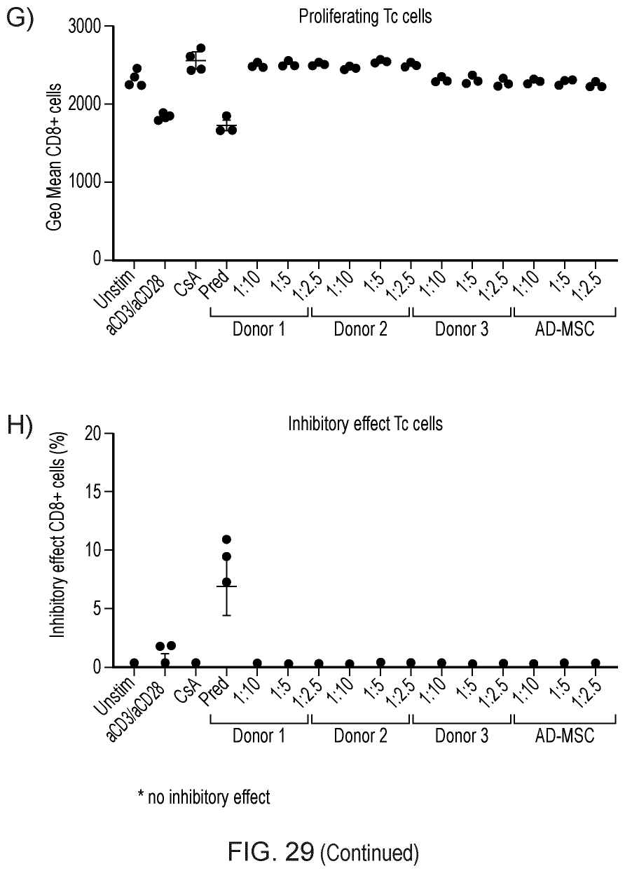

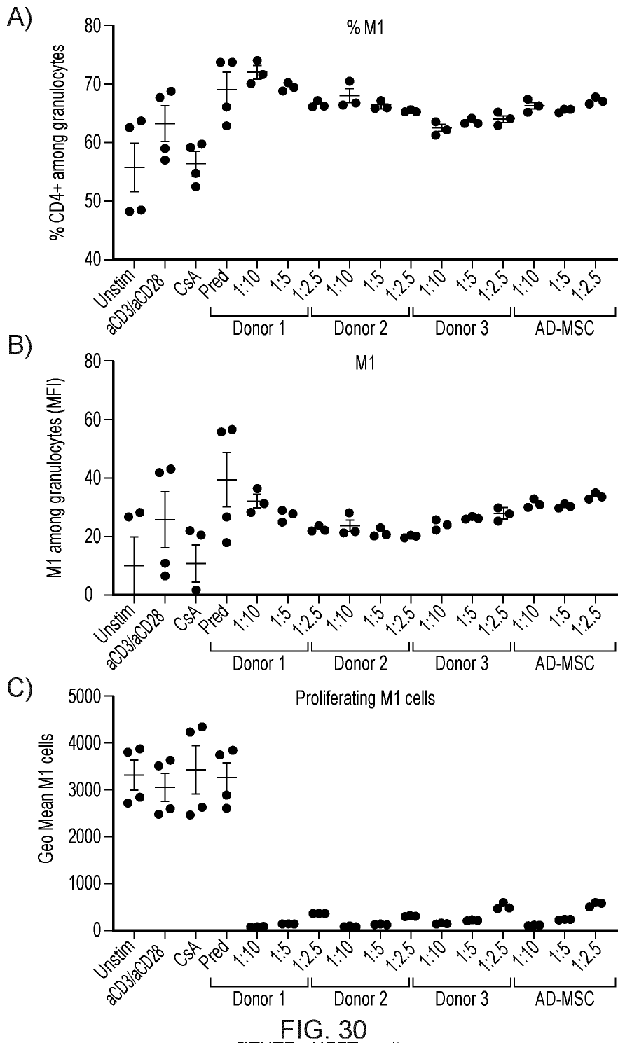

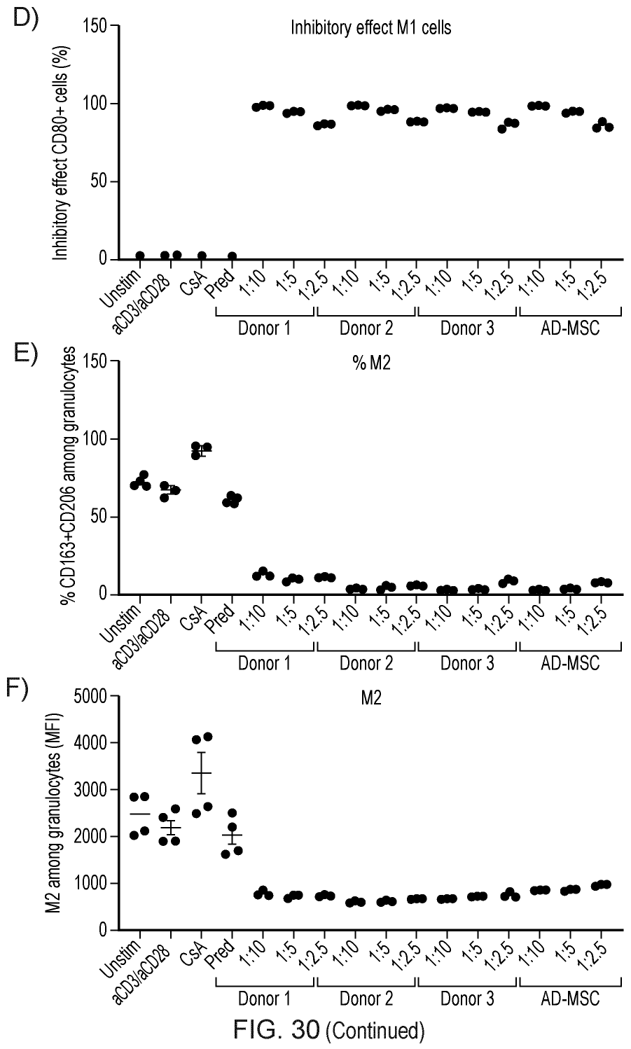

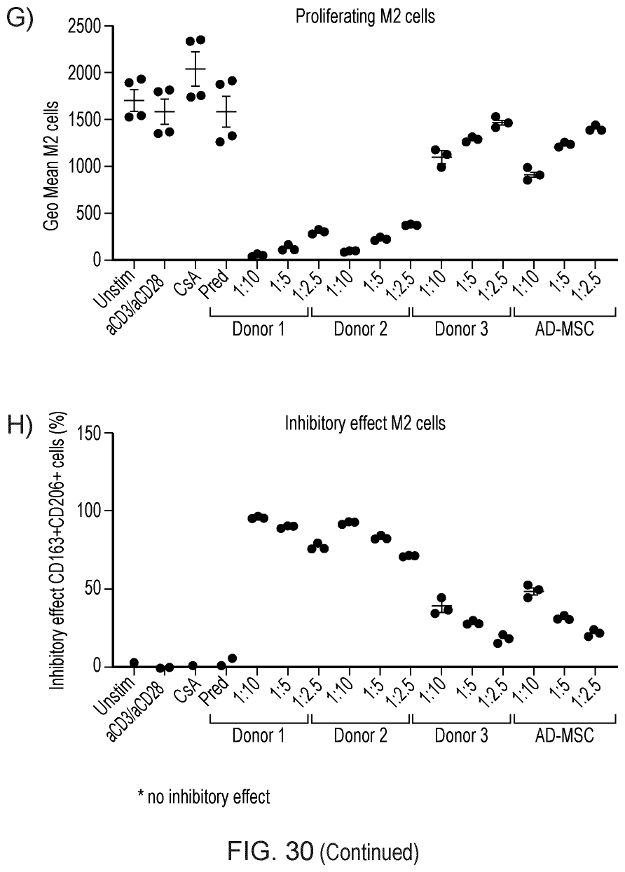

See FIG. 29-30

3.3 Cytokine Analysis after 24 Hours of Activation (Luminex)—Shown for Media 3

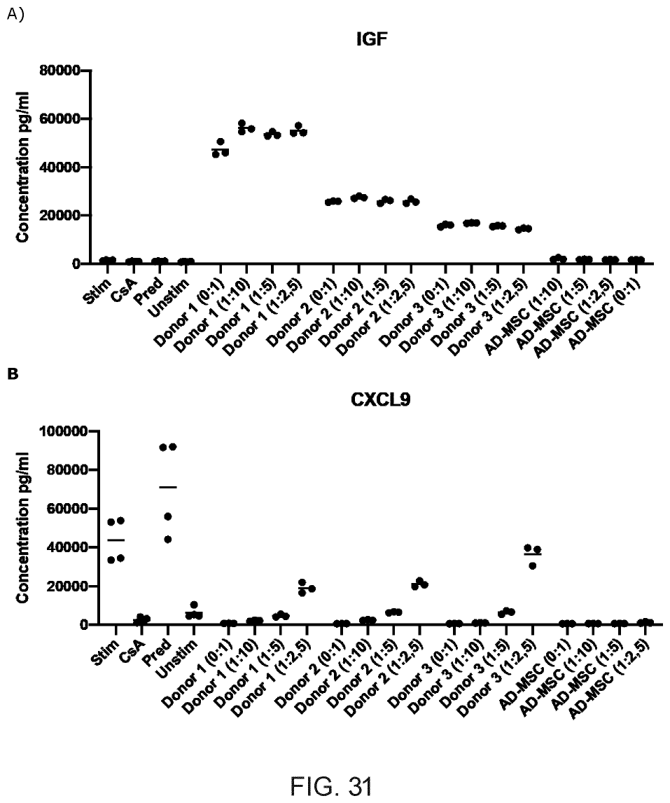

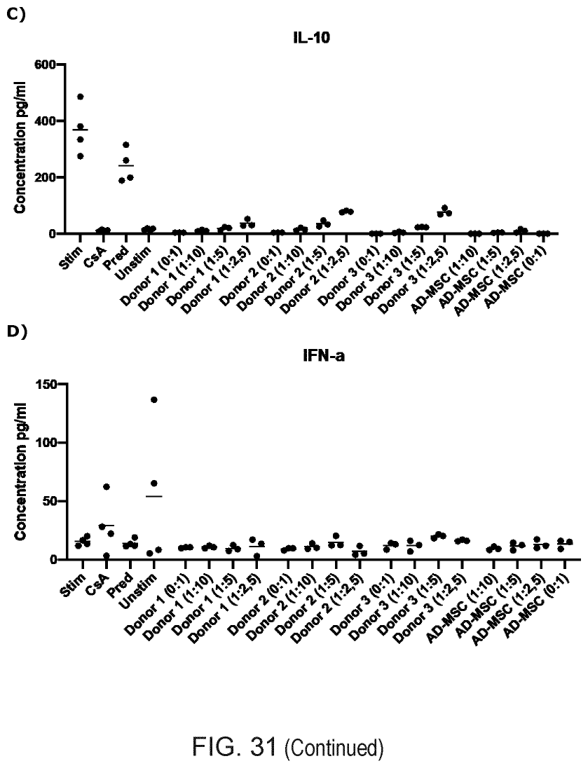

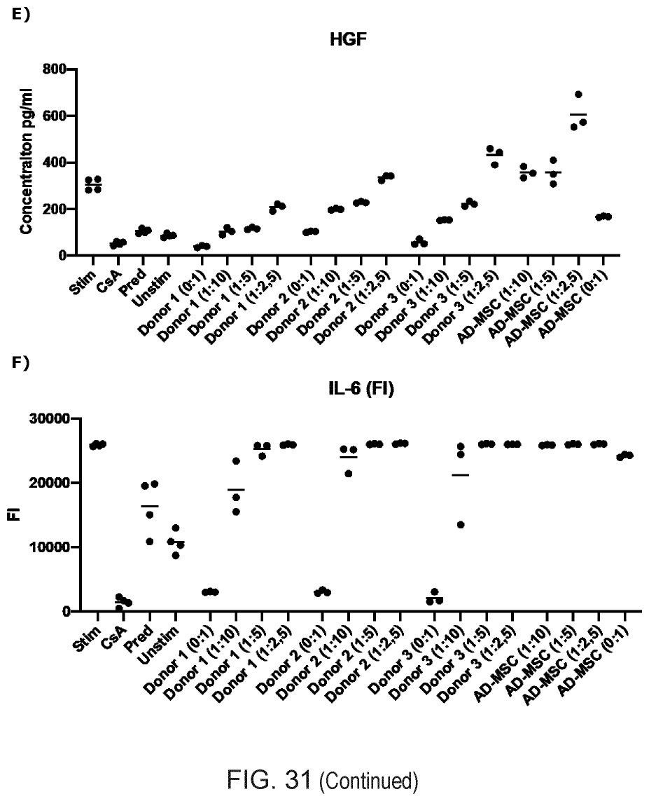

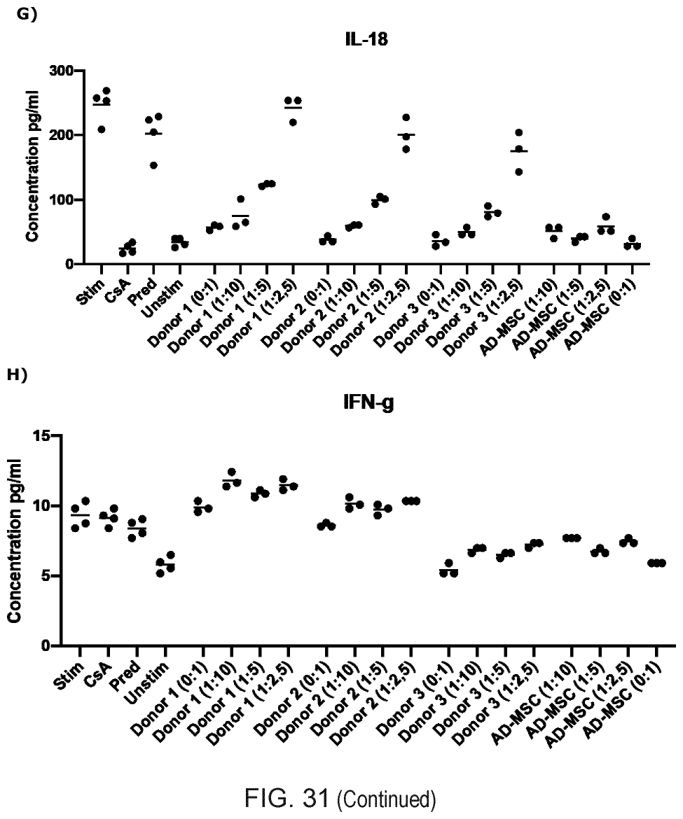

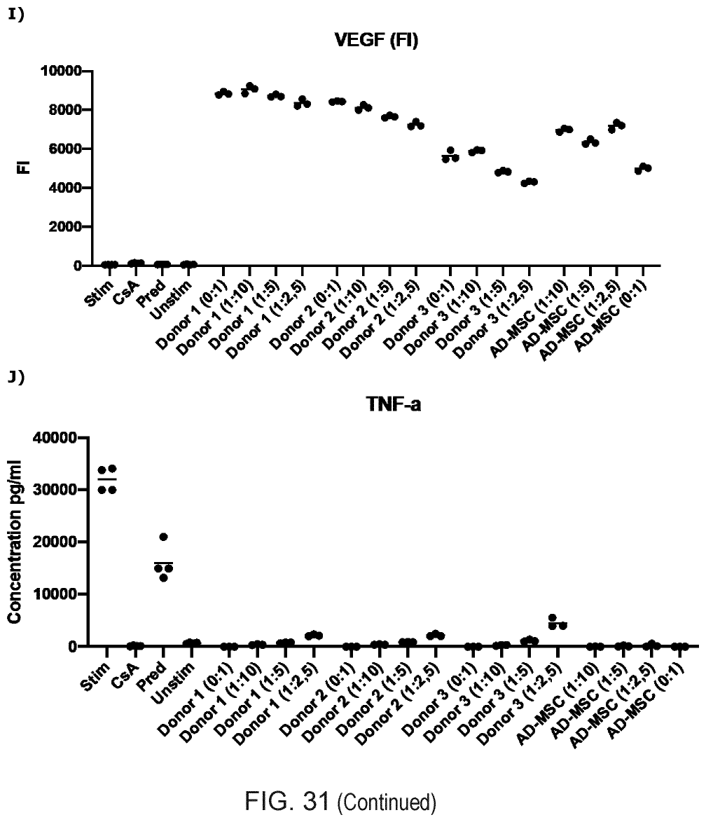

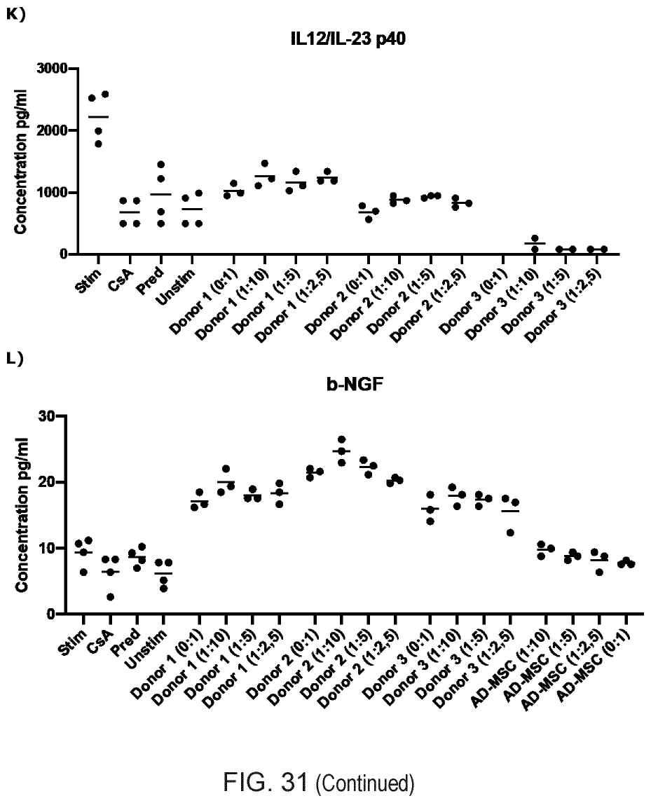

See FIG. 31

### 3.4 Luminex Analysis—CM Compared to Controls in Media 3

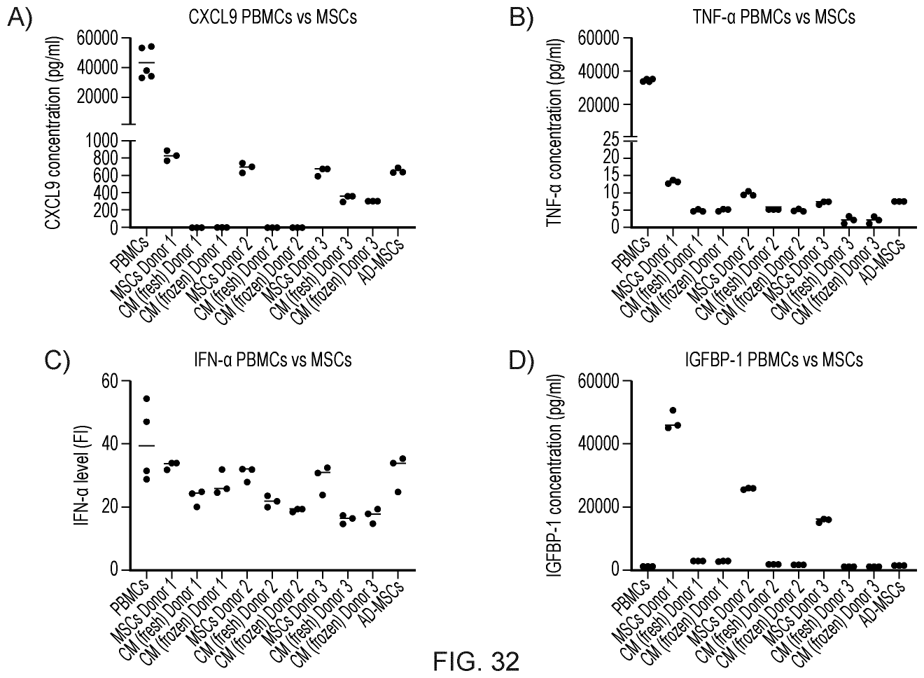

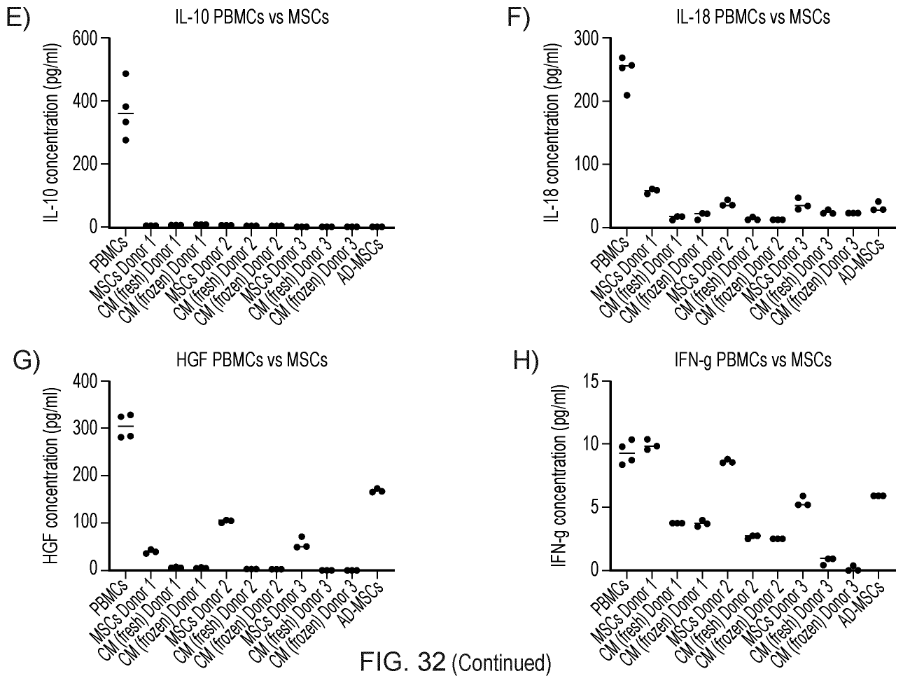

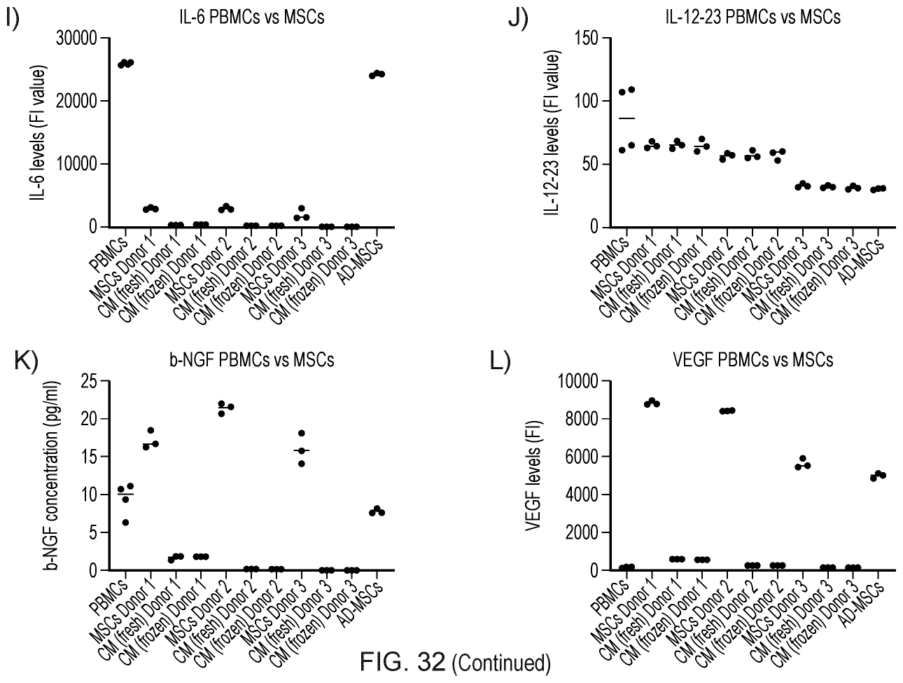

See FIG. 32

### 4. Conclusion

In this study, novel test items effect was evaluated on T cell activation and macrophage activation/polarization in human PBMCs. PBMCs were isolated from leucocyte concentrate from three different donors. Pooled PBMCs were either stained with CFSE at 5 μM for 5 minutes and added to MSCs or directly added to MSCs. Pooling of cells were performed to get a mean value from three donors but also generates a mixed lymphocyte reaction. PBMCs and MSCs were diluted in two different cell culture media (M3 and M4) at different PBMC:MSC ratios. Cell populations were analysed using flow cytometry after 24 or 72 hours. CFSE proliferation was analysed after 72 hours and cytokine levels in supernatant were analysed after 24 hours. No cut off on cell numbers were included in this analysis. Back gating was performed to verify cell populations for cells stained for cellular marker expression. For cells stained with CFSE the gates were narrowed due to unspecific leakage from channel BL1 into other channels.

PBMC:MSC ratios used in this experiment were: 1:0, 1:2.5, 1:5, 1:10 and 0:1. Thus, more MSCs than PBMCs were added per well.

PBMC:MSC ratio 0:1 in FIG. 21 shows almost no T cells or macrophages (PBMCs not added to sample) and FACS results can therefore not be compared with other ratios.

According to results in FIG. 22, showing T cell activation status after co-culturing PBMCs with MSCs or reference drugs, co-culture with PBMCs and MSCs show no effect on the composition of T helper cells (% CD4+ lymphocytes or MFI of CD4+ lymphocytes) after 24 hours of incubation (FIGS. 22A, 22B). However, results show that there is a lower percentage (8-13%) of activated T helper cells (PD-1+CD4+ lymphocytes) compared with control (23-33%) after 24 hours of incubation at all PBMC:MSC ratios (FIGS. 22C, 22D, 22E, 22F). The percentage of T regs (CD73+CD4+ lymphocytes) were higher than control after co-culturing PBMCs and MSCs at all ratios (5-10% vs 2-3%), but the expression level (MFI) of T regs were lower compared to control (FIGS. 22G, 22H, 22I, 22J). The number of T regs in ratio 0:1 is approximately 80 cells, indicating that results after co-culturing PBMCs with MSCs are correct. Results show no effect on cytotoxic T cell composition or expression level of CD8 when PBMCs were co-cultured with MSCs (% CD8+ lymphocytes or MFI of CD8+ lymphocytes) after 24 hours of incubation (FIGS. 22K, 22L). However, the percentage of activated cytotoxic T cells (% PD-1+CD8+ lymphocytes) is lower (16-25%) at all PBMC:MSC ratios compared with control (29-35%) (FIGS. 22M, 22O). Similarly, MFI values decreased from 120-170 in control to 50-60 in all PBMC:MSC ratios (FIGS. 22N, 22P). The expression level of T effector cells (CD73+CD8+ lymphocytes) is also lower at all PBMC:MSC ratios (FIGS. 22Q, 22R, 22S, 22T) compared with control (10-15% vs 23%).

In summary, co-culture of PBMCs and MSCs results in fewer activated T helper cells, more regulatory T cells, fewer activated cytotoxic T cells, and fewer effector T cells compared to PBMC controls.

According to results in FIG. 23, showing macrophage activation status, co-culture of PBMCs with MSCs results in fewer activated macrophages. The percentage of M1 macrophages (CD80+ granulocytes/macrophages) drops from 80% to 40% while MFI values drop from 120 to less than 25 (FIGS. 23A, 23B). At the same time the percentage of M2 macrophages (CD163+CD206+ granulocytes/macrophages) drops from 50% to 5-20% while MFI values drop from 2000 to less than 750 (FIGS. 23E, 23F). CD73 was almost universally expressed on activated (M1 and M2) macrophages (FIGS. 23C, 23G). However, an increased expression intensity of CD73 can be seen in both M1 (MFI 750-1250 of CD80+ macrophages compared to 600 in controls) and M2 (MFI of 800-1000 of CD163+CD206+ macrophages compared to 750 in controls) after co-culture of PBMCs and MSCs (FIGS. 23D, 23H).

Assay controls (cell composition): Prednisolone treated PBMCs show lower levels of % cytotoxic T cells (FIG. 21G) and higher levels of % T helper cells (FIG. 21A), but only slightly inhibits T cell activation (FIGS. 21C, 21D, 21I, 21J). CsA treated PBMCs show a lower expression level of activated T helper cells (FIGS. 21C, 21D) and activated cytotoxic T cells (FIGS. 21I, 21J). However, the inhibition of T cell activation by CsA was not as good as that seen with co-culture with MSCs (FIGS. 21C, 21D, 21I, 21J).

Prednisolone did not have an effect on macrophage cell composition (FIG. 23). CsA treated PBMCs show a lower expression level of M1 macrophages (FIGS. 23A, 23B), but not with as large an effect on macrophage activation as MSCs. CsA also showed a shift towards M2 macrophage expression (FIGS. 23E, 23F) and a lower expression level of CD73+M1 (FIGS. 23C, 23D) and CD73+M2 (FIGS. 23G, 23H).

In summary, co-culture of PBMCs and MSCs result in fewer activated macrophages, both of the M1 and the M2 subtypes, compared to PBMC controls. Co-culture with PBMCs and MSCs almost remove the granulocyte population indicating a reverse effect on an activated immune response. Prednisolone treated cells show no effect on the M1 or M2 expression levels although, CsA treated cells show a shift towards M2.

According to results in FIG. 29, a co-culture with PBMCs and MSCs show no effect on T helper cells. Although, a small inhibitory effect can be seen on cytotoxic T cells after 72 hours of incubation. No effect can be seen on T cell proliferation when using a co-culture with PBMCs and MSCs. As results show in FIG. 30, a co-culture with PBMCs and MSCs show a strong inhibitory effect on M2 expression levels but no effect on M1 expression levels. A strong inhibitory effect can be seen on both M1 proliferation and M2 proliferation when using a co-culture with PBMCs and MSCs.

Assay controls (CFSE proliferation): CsA and Prednisolone treated cells show no effect on % T helper cells or cytotoxic T cells after 72 hours of incubation. Prednisolone treated cells show no effect on the M1 or M2 expression levels although, CsA treated cells show a shift towards M2. No effect can be seen on T cell proliferation when using a co-culture with PBMCs and MSCs after CsA treatment. Prednisolone inhibits proliferation of cytotoxic T cells. No effect can be seen on M1 or M2 proliferation after CsA or Prednisolone treatment.

Results from cytokine analysis after co-culturing PBMCs with MSCs or reference drugs are shown in FIG. 31. Results from cytokine analysis in MSC culture supernatant (CM) or in PBMCs and MSCs not grown in co-culture are shown in FIG. 32. VEGF is produced by MSCs but not by PBMCs (FIGS. 31I, 32L). IGF and b-NGF are produced by MSCs but not by PBMCs or AD-MSCs (FIGS. 31A. 32D, 31L, 32K). IL-10, TNF-α, and IL12/IL23p40 are produced by stimulated PBMCs and downregulated in co-culture of PBMCs and MSCs at all ratios (FIGS. 31C, 31J, 31K). CXCL9, HGF and IL-18 are produced by stimulated PBMCs and downregulated in co-culture of PBMCs and MSCs in a dose-dependent manner (FIGS. 31B, 31E, 31G). Since PBMC:MSC ratios used in this experiment were: 1:2.5, 1:5, and 1:10, more MSCs than PBMCs were added per well and results from CXCL9, HGF and IL-18 are therefore difficult to interpret. No inhibitory effect can be seen on IFN-g (FIG. 31H).

No conclusions can be drawn from the figure with IFN-α (FIG. 31D). Outliers have to be removed and figure redone before analysis can be made. No conclusions can be drawn from the figure with IL-6 (FIG. 31F). It appears as if the assay sensitivity could be wrongly calibrated. It appears as if maximal values are reached for all conditions. In FIG. 321 it is shown that the pro-inflammatory cytokine IL-6 is produced by PBMCs and AD-MSCs, but not by test item MSCs.

CsA also inhibits CXCL9 (FIG. 31B), IL-10 (FIG. 31C), HGF (FIG. 31E), IL-6 (FIG. 31F), IL-18 (FIG. 31G), TNF-α (FIG. 31J) and IL-13/IL-23 p40 (FIG. 31K) levels in supernatant. No inhibitory effect can be seen on the other analysed cytokines. Prednisolone also inhibits HGF (FIG. 31E), IL-6 (FIG. 31F), TNF-α (FIG. 31J) and IL-12/IL-23 p40 (FIG. 31K) levels in supernatant. No inhibitory effect can be seen on the other analysed cytokines.

### REFERENCES

The listing or discussion of an apparently prior-published document in this specification should not necessarily be taken as an acknowledgement that the document is part of the state of the art or is common general knowledge. The references disclosed, to the extent that they provide exemplary procedural or other details supplementary to those set forth herein, are specifically incorporated herein by reference.
Cho, H., Kim, D., Kim, G., Jeong, I., 2017, Anticoagulation Therapy during Extracorporeal Membrane Oxygenator Support in Pediatric Patients, Chonnam Med J, 53(2):110-117.
Ge, J., Guo, L., Wang, S., Zhang, Y., Cai, T., Zhao, R., Wu, Y., 2014, The Size of Mesenchymal Stem Cells is a Significant Cause of Vascular Obstructions and Stroke, Stem Cell Rev and Rep, 10:295-303.
Hoogduijn, M., van den Beukel, J., Wiersma, L., Ijzer, J., 2013, Morphology and size of stem cells from mouse and whale: observational study, BMJ, 2013; 347:f6833.
Millar, J., von Bahr, V., Malfertheiner, M., Ki, K., Redd, M., Bartnikowski, N., Suen, J., McAuley, S., Fraser, J., 2019, Administration of mesenchymal stem cells during ECMO results in a rapid decline in oxygenator performance, Thorax, 74(2):194-196.
Millar, J., Bartnikowski, N., Passmore, M., Obonyo, N., Malfertheiner, M., von Bahr, V., Redd, M., Hoe, L., Ki, K., Pedersen, S., Boyle, A., Baillie, J., Shekar, K., Palpant, N., Suen, J., Matthay, M., McAuley, D., Fraser, J., 2020, Combined Mesenchymal Stromal Cell Therapy and ECMO in ARDS: A Controlled Experimental Study in Sheep, Am J Respir Crit Care Med, 202(3):383-392.
Mo, M., Zhou, Y., Li, S., Wu, Y., 2017, Three-Dimensional Culture Reduces Cell Size By Increasing Vesicle Excretion, Stem Cells, 2018(36):286-292.

1. A method of treating low blood oxygenation levels or acute respiratory distress syndrome (ARDS) in a subject via extracorporeal membrane oxygenation (ECMO) comprising:
establishing an extracorporeal blood circuit with the subject and an extracorporeal blood treatment system that comprises:
a processing fluid circuit, wherein the extracorporeal blood circuit and processing fluid circuit are divided by an oxygenation membrane of a filtration unit;
at least one blood pump for controlling the flow of blood through the blood circuit;
at least one processing fluid pump for controlling the flow of processing fluid through the processing fluid circuit;
a system computing unit operatively connected to the blood pump and the processing fluid pump, the system computing unit comprising at least one input, wherein:
the system computing unit is configured to receive a desired blood oxygenation value O_{b};
the system computing unit is configured to receive an actual blood oxygenation value Oₐ;
the system computing unit is configured to control the blood pump and the processing fluid pump so that the actual blood oxygenation value Oₐ is driven towards the desired blood oxygenation value O_{b}; and
the system is configured to receive isolated TAF MSCs; and
introducing isolated TAF MSCs or a composition that comprises isolated TAF MSCs into the extracorporeal blood treatment system.

2-19. (canceled)

20. The method according to claim 1, wherein the isolated TAF MSCs are administered before, after, and/or during ECMO.

21. The method according to claim 1, wherein a composition comprising TAF MSCs is introduced into the extracorporeal blood treatment system.

22. The method according to claim 21, wherein the composition is administered before, after, and/or during ECMO.

23. The method according to claim 1, wherein the subject is treated for ARDS.

24. The method according to claim 1, wherein the composition comprising isolated TAF MSCs is introduced into the extracorporeal blood treatment system.

25. An extracorporeal blood treatment system, comprising:
an extracorporeal blood circuit;
a processing fluid circuit, wherein the extracorporeal blood circuit and processing fluid circuit are divided by an oxygenation membrane of a filtration unit;
at least one blood pump for controlling the flow of blood through the blood circuit;
at least one processing fluid pump for controlling the flow of processing fluid through the processing fluid circuit;
a system computing unit operatively connected to the blood pump and the processing fluid pump, wherein:
the system computing unit has at least one input;
the system computing unit is configured to receive a desired blood oxygenation value O_{b};
the system computing unit is configured to receive an actual blood oxygenation value Oₐ;
the system computing unit is configured to control the blood pump and the processing fluid pump so that the actual blood oxygenation value Oₐ is driven towards the desired blood oxygenation value O_{b}; and
the system is configured to receive isolated TAF MSCs or a composition comprising isolated TAF MSCs.

26. The extracorporeal blood treatment system according to claim 25, wherein the system is configured to receive:
(i) 20 million isolated TAF MSCs per minute;
(ii) isolated TAF MSCs before and/or after the oxygenation membrane;
(iii) a desired highest blood concentration of isolated TAF MSCs;
(iv) a desired highest oxygenation membrane pressure; and
(v) an initial infusion rate of 7.5-20.0 units/kg/h of heparin.

27. The method according to claim 1, wherein the composition comprising TAF MSCs is introduced and the composition further comprises dimethyl sulfoxide (DMSO).

28. A method of oxygenating a blood sample in the presence of isolated TAF MSCs comprising contacting a blood sample with TAF MSCs or a composition comprising TAF MSCs.

29. The method according to claim 1, wherein the number of isolated TAF MSCs introduced is at least 1 million cells per kg of the subject/patient.

30. The method according to claim 1, wherein the isolated TAF MSCs are introduced before, during and/or after the blood sample contacts the membrane.

31. The method according to claim 1, further comprising introducing an anticoagulant, into the extracorporeal blood treatment system.

32. The method according to claim 31, wherein the anti-coagulant comprises a heparin.

33. The method according to claim 1, wherein the isolated TAF MSCs are:
a clonal population;
a mix of clonal populations;
heterogeneous or homogeneous;
in a single-cell suspension or pelleted;
are capable of forming colony forming units (CFU) in culture;
functionally characterised;
have been pre-sorted or enriched to contain markers of interest;
passaged; and/or
in a frozen state.

34. The method according to claim 1, wherein the isolated TAF MSCs introduced comprise:
(i) at least one surface marker selected from the group consisting of: a TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2;
(ii) at least one surface marker selected from the group consisting of PCDH19, DDR1, MME, IFITM10, BGN, NOTCH3, SULF1, TNFSF18, BDKRB1, FLT1, PDGFRA, TNFSF4, UNC5B, FAP, CASP1, CD248, DDR2, PCDH18, LRRC38, and CRLF1;
(iii) at least one surface marker selected from the group consisting of HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1;
(iv) at least one surface marker selected from the group consisting of TNFSF18, PCDH19, NCAM2, TNFSF4, CD248, DDR2, HTR2B, PCDH18, SULF1, MME, ADGRA2, DCSTAMP, PDGFRA, UNC5B, SCUBE3, CEMIP, BDKRB1, FLT1, BDKRB2, FAP, CASP1, and SRPX2; or
(v) at least one surface marker selected from the group consisting of HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1, and ITGB4.

35. The method according to claim 1, wherein the isolated TAF MSCs introduced have an average size between 15-25 μm diameter.

36. The method according to claim 1, wherein the isolated TAF MSCs introduced comprise lower actin expression or fewer vesicles at the surface compared with adult MSCs.

37. The method according to claim 1, wherein at least 10%, 20%, 30%, 40%, 50%, 60%, 70%, 80%, 90%, 95%, 96%, 97%, 98% or 99% of the TAF MSCs introduced are lung TAF MSCs.
